(19) United States
(12) Patent Application Publication
(10) Pub. No.: US 2003/0166202 A1
(43) Pub. Date: Sep. 4, 2003

(54) P450 Monooxygenases of the cyp79 family

(76) Inventors: Mette Dahl Andersen, Frederiksberg (DK); Birger Lindberg Moller, Bronshoj (DK); John Strikart Nielsen, Kastrup (DK); Ute Wittstock, Jena (DE); Carsten Horslev Hansen, Potsdam (DE); Barbara Ann Halkier, Copenhagen K (DK); Michael Dalgaard Mikkelsen, Valby (DK); Peter Kamp Busk, Soborg (DK); Soren Bak, Copenhagen N (DK)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/181,157

(22) PCT Filed: Jan. 11, 2001
(86) PCT No.: PCT/EP01/00297

(30) Foreign Application Priority Data
Jan. 13, 2000 (EP) 00100646.9
Mar. 30, 2000 (EP) 00107001.0
May 3, 2000 (EP) 00109423.4
Jul. 13, 2000 (EP) 00114184.5
Jul. 17, 2000 (EP) 00114912.9

(51) Int. Cl. C12P 13/00; C12N 9/06; C07H 21/04; C12P 21/02; C12N 5/06

(52) U.S. Cl. 435/191; 435/69.1; 435/320.1; 435/325; 536/23.2; 435/128

(57) ABSTRACT

The invention provides DNA coding for cytochrome P450 monooxygenases of the CYP79 family catalyzing the conversion of an aliphatic or aromatic amino acid or chain-elongated methionine homologue to the corresponding oxime. Preferred embodiments of the invention are enzymes catalyzing the conversion of L-Valine and L-Isoleucine such as the cassava enzymes CYP79D1 and CYP79D2, enzymes catalyzing the conversion of tyrosine such as the Triglochin maritima enzymes CYP79E1 and CYP79E2, enzymes catalyzing the conversion of tryptophan to the corresponding oxime indole-3-acetaldoxime such as the Arabidopsis thaliana enzyme CYP79A2 and the Brassica napus enzyme CYP79B5, and enzymes catalyzing the conversion of a chain-elongated methionine homologue such as the Arabidopsis thaliana enzymes CYP79F1 and CYP79F2. Transgenic expression of said DNA or parts thereof in plants can be used to manipulate the biosynthesis of corresponding glucosinolates or cyanogenic glucosides.

The present invention provides DNA coding for cytochrome P450 monooxygenases catalyzing the conversion of an aliphatic or aromatic amino acid or a chain-elongated methionine homologue to the corresponding oxime. Specific embodiments of the invention are

enzymes catalyzing the conversion of L-Valine and L-Isoleucine which belong to the new subfamily CYP79D of P450 monooxygenases such as the two cassava enzymes CYP79D1 and CYP79D2;

enzymes catalyzing the conversion of tyrosine to p-hydroxyphenylacetaldoxime which belong to the new subfamily CYP79E of P450 monooxygenases such as the two Triglochin maritima enzymes CYP79E1 and CYP79E2;

enyzmes catalyzing the conversion of L-phenylalanine to phenylacetaldoxime which belong to the subfamily CYP79A of P450 monooxygenases such as the Arabidopsis thaliana enzyme CYP79A2;

enzymes catalyzing the conversion of tryptophan to indole-3-acetaldoxime (IAOX), involved in the biosynthesis of indoleglucosinolates and possibly the biosynthesis of the plant hormone indole acetic acid (IAA), which belong to the subfamily CYP79B of P450 monooxygenases such as the Arabidopsis thaliana enzyme CYP79B2 and the Brassica napus enzyme CYP79B5; and

enyzmes catalyzing the conversion of an aliphatic amino acid or chain-elongated methionine homologue to the corresponding aldoxime which belong to the new subfamily CYP79F such as the Arabidopsis thaliana enzymes CYP79F1 and CYP79F2.

Transgenic expression of said DNA or parts thereof in plants can be used to manipulate the biosynthesis of glucosinolates or cyanogenic glucosides.

Cytochrome P450 enzymes are heme containing enzymes constituting a supergene family. In plants, they are divided into two distinct groups (Durst et al, Drug Metabolism and Drug Interact 12: 189-206, 1995). The A-group has probably been derived from a common ancestor and is involved in the biosynthesis of secondary plant products such as cyanogenic glucosides and glucosinolates. The Non A-group is heterogeneous and clusters near to animal, fungal and microbial cytochrome P450s. Cytochrome P450s showing amino acid sequence identities above 40% are grouped within the same family (Nelson et al, DNA Cell Biol. 12: 1-51, 1993). Cytochrome P450s showing more than 55% identity belong to the same subfamily.

Glucosinolates are amino acid-derived, secondary plant products containing a sulfate and a thioglucose moiety. The occurence of glucosinolates is restricted to the order Capparales and the genus Drypetes (Euphorbiales). C. papaya is the only known example of a plant containing both glucosinolates and cyanogenic glucosides. The order Capparales includes agriculturally important crops of the Brassicaceae family such as oilseed rape and Brassica forages and vegetables, and the model plant Arabidopsis thaliana L. Upon tissue damage, glucosinolates are rapidly hydrolyzed to biologically active degradation products. Glucosinolates or rather their degradation products defend plants against insect and fungal attack and serve as attractants to insects that are specialized feeders on Brassicaceae. The degradation products have toxic as well as protective effects in higher animals and humans. Antinutritional effects such as growth retardation caused by consumption of large amounts of rape seed meal have an economical impact as they restrict the use of this protein-rich animal feed. Anticarcinogenic activity has been documented by pharmacological studies for several degradation products of glucosinolates, e.g. for sulforaphane, a degradation product of 4-methylsulfinylbutylglucosinolate from broccoli sprouts. Metabolic engineering of the biosynthetic pathways of glucosinolates allows to tissue-specifically regulate and optimize the level of individual glucosinolates to improve the nutritional value of a given crop. Besides their occurrence in A. thaliana, such glucosinolates are important constituents of Brassica crops and vegetables. For example, the major glucosinolate in B. napus, the goitrogenic 2-hydroxy-3-butenylglucosinolate, is formed by side-chain modification of 4-methylthiobutylglucosinolate. The occurrence of 2-hydroxy-3-butenylglucosinolate in B. napus restricts the use of the protein-rich seed cake as animal feed. Thus availability of biosynthetic genes has great potential for the development of crops with reduced levels of undesirable glucosinolates while retaining glucosinolates with desirable effects, e.g. for pest resistance.

To date, more than 100 different glucosinolates have been identified. They are grouped into aliphatic, aromatic, and indolyl glucosinolates, depending on whether they are derived from aliphatic amino acids, phenylalanine and tyrosine, or tryptophan. The amino acid often undergoes a series of chain elongations prior to entering the biosynthetic pathway, and the glucosinolate product is often subject to secondary modifications such as hydroxylations, methylations, and oxidations giving rise to the structural diversity of glucosinolates.

Arabidopsis thaliana cv. Columbia has been shown to contain 23 different glucosinolates derived from tryptophan, the chain-elongated phenylalanine homologue homophenyl-alanine, and several chain-elongated methionine homologues such as dihomo-, trihomo- and tetrahomomethionine.

In the present invention we have identified amongst others a CYP79 homologue, CYP79B2 from Arabidopsis, which catalyzes the conversion of tryptophan to IAOX, a precursor for the biosynthesis of both indoleglucosinolates and the plant hormone IAA. Overexpression of CYP79B2 in Arabidopsis results in an increased level of indoleglucosinolates, which shows that CYP79B2 is involved in biosynthesis of indoleglucosinolates and that the evolution of indoleglucosinolates is based on a ‘cyanogenic’ predisposition.

Not many genes of the glucosinolate biosynthetic pathway have been identified. The nature of the enzymes catalyzing the conversion of amino acids to aldoximes has been the subject of many discussions. Independent biochemical studies have indicated that three different enzyme systems are involved in this step, namely cytochrome P450-dependent monooxygenases, flavin-containing monooxygenases, and peroxidases. Based on microsomal enzyme preparations from species of the Brassicaceae it has previously been proposed, that the conversion of dihomo-, trihomo- and tetrahomomethionine to their corresponding aldoximes is catalyzed by flavin-containing monooxygenases.

In the biosynthesis of cyanogenic glucosides, cytochromes P450 of the CYP79 family catalyze the formation of aldoximes from amino acids. For example the aromatic amino acid precursor L-tyrosine is hydroxylated twice by the enzyme CYP79A1 (P450_{TYR}) forming (Z)-p-hydroxyphenylacetaldoxime (WO 95/16041), which subsequently is converted by the enzyme CYP71 E1 (P450_{OX}) to the cyanohydrine p-hydroxymandelonitrile (WO 98/40470). p-hydroxymandelonitrile is finally conjugated to glucose by a UDP-glucose:aglycon-glucosyltransferase. Transgenic expression of said enzymes can be exploited to modify, reconstitute, or newly establish the biosynthetic pathway of cyanogenic glucosides or to modify glucosinolate production in plants. Several CYP79 homologues have been identified in glucosinolate-producing plants, but their function has never been determined. The present invention discloses cloning and functional expression of the cytochromes P450 CYP79A2, CYP79B2 and CYP79F1 from A. thaliana as well as cloning of the cytochrome P450 CYP79B5 from Brassica napus. It shows that CYP79A2 catalyzes the conversion of L-phenylalanine to phenylacetaldoxime, CYP79B2 the conversion of tryptophan to indole-3-acetaldoxime, and CYP79F1 the conversion of chain-elongated methionine homologues such as e.g. homo-, dihomo-, trihomo-, tetrahomo-, pentahomo- and hexahomomethionine to their corresponding aldoximes. It further shows that transgenic A. thaliana expressing CYP79A2 or CYP79B2 under control of the CaMV35S promoter accumulate high levels of benzyl- or indoleglucosinolates, respectively, whereas transgenic Arabidopsis thaliana expressing CYPF1 can show cosuppression of CYPF1 with a reduced content of glucosinolates derived from chain-elongated methionine homologues and with highly increased levels of chain-elongated methionines such as e.g. dihomo- and trihomomethionine. The data are consistent with the involvement of CYP79A2, CYP79B2 and CYP79F1 in the glucosinolate biosynthesis in A. thaliana. The presence of an IAOX producing CYP79 in the biosynthesis of indoleglucosinolates is unexpected since no tryptophan-derived cyanogenic glucosides have been identified and a peroxidase activity has been described in the literature as being involved in indoleglucosinolate biosynthesis. Furthermore, indoleglucosinolates are the products of a recent evolutionary event and are present only in four families in the Capparales order, namely in Brassicaceae, Resedaceae, Tovariaceae and Capparaceae. Thus, the possible involvement of IAOX in the biosynthesis of both IAA and indoleglucosinolates would suggest that the nature of the enzyme catalyzing the conversion of tryptophan to IAOX is different from a CYP79 N-hydroxylase. The characterization of CYP79B2 in planta as well as in vitro demonstrates, that oxime production by CYP79 proteins in the biosynthesis of glucosinolates is not restricted to those aromatic amino acids that are also precursors in cyanogenic glucoside biosynthesis. This shows that after diverging away from cyanogenic glucosides, CYP79 proteins developed a new substrate specificity. As a consequence thereof, it is expected that a number of cytochrome P450s of glucosinolate producing plants belonging to the CYP79 family, will turn out to catalyze oxime production from various precursor amino acids in glucosinolate biosynthesis.

Cassava, the most important tropical root crop, contains two cyanogenic glucosides, i.e. linamarin and lotaustralin, in all parts of the plant. Upon tissue disruption said glucosides are degraded with concomitant release of hydrogen cyanide. Acyanogenic cassava plants are not known and attempts to completly eliminate cyanogenic glucosides through breeding have not been successful. Thus, use of cassava products as staple food requires careful processing to remove the cyanide. Processing, however, is labor intensive, time-consuming and results in the simultaneous loss of proteins, vitamins and minerals. Identification of enzymes involved in the biosynthetic pathway of linamarin and lotaustralin would open the door to molecular biological approaches to suppress the biosynthesis of said cyanogenic glucosides such as sense or antisense suppression.

Triglochin maritima (seaside arrow grass) contains two cyanogenic glucosides, i.e. taxiphyllin and triglochinin, in most parts of the plant. Upon tissue disruption said glucosides are degraded with concomitant release of hydrogen cyanide. Acyanogenic seaside arrow grass is not known. Identification of enzymes involved in the biosynthetic pathway of taxiphyllin, the epimer of dhurrin, and triglochinin and the corresponding cDNA or genomic clones allow molecular biological approaches to suppress the biosynthesis of said cyanogenic glucosides such as sense or antisense suppression or to select desired alterations using marker assisted selection. Though it is tempting to infer the involvement of analogous multifunctional cytochrome P450 enzymes from a common biosynthetic route for cyanogenic glucoside biosynthesis in a number of different plant species this may not be so in Triglochin maritima, since in this plant p-hydroxyphenylacetonitrile is free to equilibrate. The cytochrome P450 catalyzed conversion of aldoxime to nitrile is a dehydration reaction and as such unusual. In Triglochin maritima it might be carried out by an additional enzyme activity associated with the first multifunctional cytochrome P450 enzyme instead of being the first catalytic event catalyzed by the second cytochrome P450 involved. If so, the second cytochrome P450 in Triglochin maritima would constitute a usual C-hydroxylase.

Gene refers to a coding sequence and associated regulatory sequences wherein the coding sequence is transcribed into RNA such as mRNA, rRNA, tRNA, snRNA, sense RNA or antisense RNA. Examples of regulatory sequences are promoter sequences, 5′ and 3′ untranslated sequences and termination sequences. Further elements such as introns may be present as well.

Expression generally refers to the transcription and translation of an endogenous gene or transgene in plants. However, in connection with genes which do not encode a protein such as antisense constructs, the term expression refers to transcription only.

The following solutions are provided by the present invention:

A DNA coding for a P450 monooxygenase converting an aliphatic or aromatic amino acid or chain-elongated methionine homologue, such as valine, leucine, isoleucine, cyclopentenylglycine, tyrosine, L-phenylalanine, tryptophan, dihomo-, trihomo- or tetrahomomethionine to the corresponding oxime;

Said DNA coding for a P450 monooxygenase, wherein global alignment of the amino acid sequence of the encoded protein shows at least 40% identity to the amino acid sequence resulting from the global alignment with SEQ ID NO: 1 or SEQ ID NO: 3 or both; SEQ ID NO: 39; or SEQ ID NO: 54 or SEQ ID NO: 70 or both; or at least 50% identity to the amino acid sequence resulting-from the global alignment with SEQ ID NO: 9 or SEQ ID NO: 11 or both or SEQ ID NO: 74 or SEQ ID NO: 84 or both.

Said DNA coding for a P450 monooxygenase having the formula R₁-R₂-R₃, wherein

R₁, R₂ and R₃ designate component sequences, and

R₂ consists of 150 to 175 or more amino acid residues the sequence of which is at least 60% identical to an aligned component sequence of SEQ ID NO: 1 or SEQ ID NO: 3; SEQ ID NO: 9 or SEQ ID NO: 11; SEQ ID NO: 54 or SEQ ID NO: 70; SEQ ID NO: 74 or SEQ ID NO: 84; or at least 65% identical to an aligned component sequence of SEQ ID NO: 39.

A P450 monooxygenase converting an aliphatic or aromatic amino acid or a chain-elongated methionine homologue to the corresponding oxime;

A method for the isolation of a cDNA coding for a P450 monooxygenase converting an aliphatic or aromatic amino acid or chain-elongated methionine homologue to the corresponding oxime;

A method for producing purified recombinant P450 monooxygenase converting an aliphatic or aromatic amino acid or chain-elongated methionine homologue to the corresponding oxime; and

A marker assisted breeding method using at least one oligonucleotide of at least 15 to 20 nucleotides length constituting a component sequence of the DNA according to the present invention, and

A method for obtaining a transgenic plant comprising stably integrated into its genome DNA comprising at least part of an open reading frame of a P450 monooxygenase converting an aliphatic or aromatic amino acid or chain-elongated methionine homologue to the corresponding oxime. Dependent on the constructs used resulting plants show an altered content or profile of cyanogenic glucosides or glucosinolates.

The biosynthesis of cyanogenic glucosides is believed to proceed according to a general pathway, i.e. involving the same type of intermediates in all plants. This has been clearly demonstrated for the part of the pathway involving conversion of amino acids to oximes. In all plants tested said part of the pathway is catalyzed by one or more cytochrome P450 enzymes belonging to the CYP79 family. The members of said family are proteins showing more than 40% sequence identity at the amino acid level, members showing less than 55% sequence identity are grouped in different subfamilies. For example the Sorghum enzyme catalyzing the conversion of the aromatic amino acid L-tyrosine to the corresponding oxime belongs to the subfamily CYP79A and is designated CYP79Al. The biosynthetic pathway of taxiphyllin and triglochinin also start with the conversion of the aromatic amino acid L-tyrosine to p-hydroxyphenylacetaldoxime. The biosynthetic pathway of linamarin and lotaustralin is believed to start with the conversion of the aliphatic amino acids L-Valine or L-isoleucine to the corresponding oximes.

The aim of the present invention is to provide DNA coding for P450 monooxygenases catalyzing the conversion of an aliphatic or aromatic amino acid or a chain-elongated methionine homologue to the corresponding oxime and to define their general structure on the basis of the amino acid sequence of the enzymes and corresponding gene sequences expressed in cassava, Triglochin maritima, Arabidopsis thaliana, or Brassica napus. It is found that

enzymes catalyzing the conversion of an aliphatic amino acid constitute a new subfamily of P450 enyzmes which is designated CYP79D;

enzymes catalyzing the conversion of an aromatic amino acid constitute a new subfamily of P450 enyzmes which is designated CYP79E;

enzymes catalyzing the conversion of L-phenylalanine to phenylacetaldoxime belong to the subfamily of CYP79A;

enzymes catalyzing the conversion of tryptophan to indole-3-acetaldoxime belong to the subfamily of CYP79B; and

enzymes catalyzing the conversion of an aliphatic amino acid or chain-elongated methionine homologue belong to the subfamily of CYP79F.

Thus the present invention discloses a P450 monooxygenase converting an aliphatic amino acid such as valine, leucine, isoleucine or cyclopentenylglycine to the corresponding oxime. The enzyme is specific for L-amino acids. It consists of amino acid residues independently selected from the group of the amino acid residues Gly, Ala, Val, Leu, Ile, Phe, Pro, Ser, Thr, Cys, Met, Trp, Tyr, Asn, Gln, Asp, Glu, Lys, Arg and His, and shows at least 40%, preferably 55%, or even more preferably 70% identity to the amino acid sequence resulting from global alignment with either SEQ ID NO: 1 (CYP79D1) or SEQ ID NO: 3 (CYP79D2) or both, which sequences define specific embodiments of the present invention naturally expressed in cassava. The present invention further discloses a P450 monooxygenase converting an aromatic amino acid such as tyrosine or phenylalanine to the corresponding oxime. The enzyme is specific for L-amino acids. It consists of amino acid residues independently selected from the group of the amino acid residues Gly, Ala, Val, Leu, Ile, Phe, Pro, Ser, Thr, Cys, Met, Trp, Tyr, Asn, Gln, Asp, Glu, Lys, Arg and His, and shows at least 50%, preferably 55%, or even more preferably 70% identity to the amino acid sequence resulting from global alignment with either SEQ ID NO: 9 (CYP79E1) or SEQ ID NO: 11 (CYP79E2) or both, which sequences define specific embodiments of the present invention naturally expressed in Triglochin maritima. The present invention further discloses a P450 monooxygenase converting L-phenylalanine to phenylacetaldoxime. It consists of amino acid residues independently selected from the group of the amino acid residues Gly, Ala, Val, Leu, Ile, Phe, Pro, Ser, Thr, Cys, Met, Trp, Tyr, Asn, Gln, Asp, Glu, Lys, Arg and His, and shows at least 40%, preferably 55%, or even more preferably 70% identity to the amino acid sequence resulting from global alignment with SEQ ID NO: 39 (CYP79A2), which defines a specific embodiment of the present invention naturally expressed in Arabidopsis thaliana. The present invention further discloses a P450 monooxygenase converting tryptophan to indole-3-acetaldoxime. It consists of amino acid residues independently selected from the group of the amino acid residues Gly, Ala, Val, Leu, Ile, Phe, Pro, Ser, Thr, Cys, Met, Trp, Tyr, Asn, Gln, Asp, Glu, Lys, Arg and His, and shows at least 40%, preferably 55%, or even more preferably 70% identity to the amino acid sequence resulting from global alignment with SEQ ID NO: 54 (CYP79B2)) or SEQ ID NO: 70 (CYP79B5), which define specific embodiments of the present invention naturally expressed in Arabidopsis thaliana and Brassica napus, respectively. The present invention further discloses a P450 monooxygenase converting an aliphatic amino acid or chain-elongated methionine homologue to the corresponding aldoxime. It consists of amino acid residues independently selected from the group of the amino acid residues Gly, Ala, Val, Leu, Ile, Phe, Pro, Ser, Thr, Cys, Met, Trp, Tyr, Asn, Gln, Asp, Glu, Lys, Arg and His, and shows at least 50%, preferably 55%, or even more preferably 70% identity to the amino acid sequence resulting from global alignment with SEQ ID NO: 74 (CYP79F1) or SEQ ID NO: 84 (CYP79F2), which define specific embodiments of the present invention naturally expressed in Arabidopsis thaliana.

Examples of amino acid residues which might result from posttranslational modification within a living cell are glycosylated residues of the above-mentioned amino acids as well as Aad, bAad, bAla, Abu, 4Abu, Acp, Ahe, Aib, bAib, Apm, Dbu, Des, Dpm, Dpr, EtGly, EtAsn, Hyl, aHyl, 3Hyp, 4Hyp, Ide, alle, MeGly, MeIle, MeLys, MeVal, Nva, Nle or Orn.

The amino acid sequence of the enzyme according to the invention can be further defined by the formula R₁-R₂-R₃, wherein

R₁, R₂ and R₃ designate component sequences, and

R₂ consists of 150, 175, 200 or more amino acid residues the sequence of which is at least 60% or 65%, preferably at least 70%, and even more preferably at least 75%, identical to an aligned component sequence of SEQ ID NO: 1 or SEQ ID NO: 3; SEQ ID NO: 9 or SEQ ID NO: 11; SEQ ID NO: 39; SEQ ID NO: 54 or SEQ ID NO: 70; SEQ ID NO: 74 or SEQ ID NO: 84.

Typically R₂ consists of 150 to 175 or more amino acid residues. Specific embodiments of R₂ are represented by

amino acids 334-484 of SEQ ID NO: 1 and amino acids 333-483 of SEQ ID NO: 3;

amino acids 339-489 of SEQ ID NO: 9 and amino acids 332-482 of SEQ ID NO: 11;

amino acids 308-487 of SEQ ID NO: 39;

amino acids 196-345 of SEQ ID NO: 54 and amino acids 192-341 of SEQ ID NO: 70;

amino acids 334-483 of SEQ ID NO: 74 and amino acids 332-481 of SEQ ID NO: 84.

The monooxygenase encoded by said DNA generally consist of 450 to 600 amino acid residues. Thus the specific embodiments of CYP79D1 (SEQ ID NO: 1), CYP79D2 (SEQ ID NO: 3), CYP79E1 (SEQ ID NO: 9), CYP79E2 (SEQ ID NO: 11), CYP79A2 (SEQ ID NO: 39), CYP79B2 (SEQ ID NO: 54), CYP79B5 (SEQ ID NO: 70); CYP79F1 (SEQ ID NO: 74) and CYP79F2 (SEQ ID NO: 84) have a size of 541, 542, 540, 533, 523, 541, 540, 537 and 535 amino acid residues, respectively.

In general there exist two approaches towards sequence alignment. Dynamic programming algorithms as proposed by Needleman and Wunsch and by Sellers align the entire length of two sequences providing a global alingment of the sequences. The Smith-Waterman algorithm on the other hand yields local alignments. A local alignment aligns the pair of regions within the sequences that are most similiar given the choice of scoring matrix and gap penalties. This allows a database search to focus on the most highly conserved regions of the sequences. It also allows similiar domains within sequences to be identified. To speed up alignments using the Smith-Waterman algorithm programs such as BLAST (Basic Local Alignment Search Tool) and FASTA place additional restrictions on the alignments.

Within the context of the present invention global sequence alignments are conveniently performed using the program PILEUP available from the Genetic Computer Group, Madison, Wis. Local alignments are performed conveniently using BLAST, a set of similarity search programs designed to explore all of the available sequence databases regardless of whether the query is protein or DNA. Version BLAST 2.0 (Gapped BLAST) of this search tool has been made publicly available on the internet (currently http://www.ncbi.nlm.nih.gov/BLAST/). It uses a heuristic algorithm which seeks local as opposed to global alignments and is therefore able to detect relationships among sequences which share only isolated regions. The scores assigned in a BLAST search have a well-defined statistical interpretation. Particularly useful within the scope of the present invention are the blastp program allowing for the introduction of gaps in the local sequence alignments and the PSI-BLAST program, both programs comparing an amino acid query sequence against a protein sequence database, as well as a blastp variant program allowing local alignment of two sequences only. Said programs are preferably run with optional parameters set to the default values.

Additionally, sequence alignments using BLAST can take into account whether the substitution of one amino acid for another is likely to conserve the physical and chemical properties necessary to maintain the structure and function of a protein or is more likely to disrupt essential structural and functional features. Such sequence similarity is quantified in terms of a percentage of ‘positive’ amino acids, as compared to the percentage of identical amino acids and can help assigning a protein to the correct protein family in border-line cases.

P450 monooxygenases converting an aliphatic or aromatic amino acid or a chain-elongated methionine homologue to the corresponding oxime can be purified from plants expressing said enzymes essentially as described for P450_{TYR} in example 3 of WO 95/16041.

Purified recombinant P450 monooxygenase converting an aliphatic or aromatic amino acid or a chain-elongated methionine homologue to the corresponding oxime can be obtained by a method comprising expression of the cDNA clone in yeasts such as the methylotropic yeast Pichia pastoris. To optimize expression conditions, it may be desirably to remove the 5′- and 3′-untranslated regions before insertion into an expression vector. An optimal translation initiation context can be obtained by positioning the start ATG exactly as the start ATG of the highly expressed P. pastoris AOX1 gene. Metabolic activity can be measured in intact cells because the endogenous P. pastoris reductase system is able to support electron donation to many plant cytochromes P450. To further optimize expression and enzyme activity levels a number of different growth media and growth periods can be tested including but not limited to the use of rich media and induction at about OD₆₀₀ of 0.5 for 24-30 h. The cytochrome P450 produced may be isolated from P. pastoris microsomes using initial solubilization with a detergent like Triton X-114 followed by temperature induced phase partitioning. Final purification may be achieved using ion exchange or dye column chromatography. An appropriate column for ion exchange chromatography is EAE-Sepharose FF. Appropriate columns for dye chromatography are Reactive Red 120 Agarose, Reactive Yellow 3A Agarose, or Cibachron Blue Agarose. The dye columns are conveniently eluted with KCl gradients. Fractions containing active cytochrome P450 enzymes may be identified by carbon monoxide difference spectroscopy, substrate binding spectra or by activity measurements using aliphatic or aromatic amino acids or chain-elongated methionine homologues as substrates and reconstituted cytochrome P450 enzymes.

If the endogenous P. pastoris reductase is not able to support electron donation, the recombinant protein may be isolated and reconstituted in artificial lipid micelles (Sibbesen et al, J. Biol. Chem. 270: 3506-3511, 1995; Halkier et al, Arch. Biochem. Biophys 322: 369-377, 1995; Kahn et al, Plant Physiol 115: 1661-1670, 1997) with the NADPH-cytochrome P450 oxidoreductase isolated from sorghum or from the same plant species that provided the source for the cytochrome P450 enzyme according to standard proceedures (Sibbesen et al, J. Biol. Chem. 270: 3506-3511, 1995).

Alternatively bacteria like Escherichia coli can be used for the recombinant expression of cytochrome P450 enzymes belonging to the CYP79 family. The resulting proteins are unglycosylated. Depending on the particular enzyme studied vector constructs with inserts encoding native or various truncated, extended or modified amino terminal sequences are preferred (Halkier et al, Arch. Biochem. Biophys. 322: 369-377, 1995; Barnes et al, Proc. Natl. Acad. Sci. USA 88: 5597-5601, 1991; Gillem et al, Arch Biochem Biophys 312: 59-66, 1994). A particularly preferred E. coli strain is strain C43(DE3) known to grow well while expressing a heterologous membrane protein in amounts which hold growth of commonly used strains. Thus, expression of CYP79B2 in the commonly used E. coli strain JM109 produced less than 0.5% of the CYP79B2 activity produced by strain C43(DE3). Expression in insect cells is also possible.

Investigations into the substrate specificity of CYP79D1, CYP79D2, CYP79E1, CYP79E2, CYP79A2, CYP79B2, CYP79B5 and CYP79F1 are carried out in E. coli spheroplasts reconstituted with sorghum NADPH-cytochrome P450 oxidoreductase in the presence of high amounts of lipids. L-α-dioleyl phosphatidyl choline and L-α-dilauroyl phosphatidyl choline are preferred lipids for the reconstitution. Both CYP79D1 and CYP79D2 are found to convert L-valine as well as L-isoleucine into their corresponding oximes. Both CYP79E1 and CYP79E2 are found to convert L-tyrosine into the corresponding oxime. CYP79A2 is found to convert L-phenylalanine into phenylacetaldoxime. CYP79B2 is found to convert tryptophan into indole-3-acetaldoxime. CYP79F1 is found to convert a chain-elongated methionine homologue into the corresponding aldoxime. Neither L-Leucine, L-phenylalanine nor L-tyrosine are metabolized by CYP79D1 or CYP79D2. Neither L-methionine, L-tryptophane nor L-tyrosine are metabolized by CYP79A2. Neither phenylalanine nor tyrosine are metabolized by CYP79B2. Neither L-tryptophane, L-phenylalanine nor L-tyrosine are metabolized by CYP79F1. D-Amino acids are not converted into oximes by CYP79D1, CYP79D2, CYP79E1 and CYP79E2. Depending on the nature of the substrate, substrate specificity may also be determined using intact P. pastoris cells or intact E. coli cells.

The ability of a P450 monooxygenase to convert an aliphatic or aromatic amino acid or chain-elongated methionine homologue to the corresponding oxime can be tested in an assay (see also example 5) comprising

a) incubating a reaction mixture comprising the P450 monooxygenase of the present invention or spheroplasts of E.coli cells expressing said enzyme, the parent amino acid, NADPH, oxygen, NADPH-cytochrome P450 oxidoreductase and lipid at ambient temperature for a certain period of time which is between 2 min and 2 to 6 hours;

b) terminating the reaction for example by the addition of a denaturing compounds such as ethyl acetate; and

c) chemically identifying and quantifying the aldoxime produced.

The present invention also provides nucleic acid compounds comprising an open reading frame encoding the novel proteins according to the present invention. Said nucleic acid molecules are structurally and functionally similar to nucleic acid molecules obtainable from plants producing similar biosynthetic enzymes. In a preferred embodiment of the invention an open reading frame is operably linked to one or more regulatory sequences different from the regulatory sequences associated with the genomic gene containing the exons of the open reading frame and said nucleic acid molecules hybridize to a fragment of the DNA molecule defined by SEQ ID NO: 2 or SEQ ID NO: 4; SEQ ID NO: 10 or SEQ ID NO: 12; SEQ ID NO: 40; SEQ ID NO: 55 (corresponding to the Arabidopsis cDNA encoding CYP79B2), SEQ ID NO: 56 (corresponding to Arabidopsis genomic DNA encoding CYP79B2) or SEQ ID NO: 71 (corresponding to Brassica cDNA encoding CYP79B5); or SEQ ID NO: 75 or SEQ ID NO: 85. Said fragment is more than 20 nucleotides long and preferably longer than 25, 30, or 50 nucleotides. Factors that affect the stability of hybrids determine the stringency of hybridization conditions and can be measured in dependence of the melting temperature Tₘ of the hybrids formed. The calculation of Tₘ is desribed in several textbooks. For example Keller et al describe in: “DNA Probes: Background, Applications, Procedures”, Macmillan Publishers Ltd, 1993, on pages 8 to 10 the factors to be considered in the calculation of Tₘ values for hybridization reactions. The DNA molecules according to the present invention hybridize with a fragment of SEQ ID NO: 2 or SEQ ID NO: 4; SEQ ID NO: 10 or SEQ ID NO: 12; SEQ ID NO: 40; SEQ ID NO: 55, SEQ ID NO: 56 or SEQ ID NO: 71; or SEQ ID NO: 75 or SEQ ID NO: 85 at a temperatur 30° C. below the calculated Tₘ of the hybrid to be formed. Preferably they hybridize at temperatures 25, 20, 15, 10, or 5° C. below the calculated Tₘ.

Nucleic acid compounds according to the invention consist of nucleotide residues independently selected from the group of the nucleotide residues G, A, T and C or the group of nucleotide residues G, A, U and C and are characterized by the formula R_{A}-R_{B}-R_{C}, wherein

R_{A}, R_{B} and R_{C} designate component sequences; and

R_{B} consists of at least 450 and preferably 600 or more nucleotide residues encoding amino acid component sequence R₂ as described above.

Knowledge of SEQ ID NO: 1, SEQ ID NO: 2, SEQ ID NO: 3, and SEQ ID NO: 4; SEQ ID NO: 9, SEQ ID NO: 10, SEQ ID NO: 11, and SEQ ID NO: 12; SEQ ID NO: 39 and SEQ ID NO: 40; SEQ ID NO: 54, SEQ ID NO: 55, SEQ ID NO: 56, SEQ ID NO: 70 and SEQ ID NO: 71; and SEQ ID NO: 74, SEQ ID NO: 75, SEQ ID NO: 84 and SEQ ID NO: 85 can be used to accelerate the isolation and production of DNA coding for a P450 monooxygenase converting an aliphatic or aromatic amino acid or chain-elongated methionine homologue to the corresponding aldoxime which method comprises

(a) preparing a cDNA library from plant tissue expressing such a monooxygenase,

(b) using at least one oligonucleotide designed on the basis of SEQ ID NO: 1, SEQ ID NO: 2, SEQ ID NO: 3, or SEQ ID NO: 4; SEQ ID NO: 9, SEQ ID NO: 10, SEQ ID NO: 11, or SEQ ID NO: 12;; SEQ ID NO: 39 and SEQ ID NO: 40; SEQ ID NO: 54, SEQ ID NO: 55, SEQ ID NO: 56, SEQ ID NO: 70 or SEQ ID NO: 71; or SEQ ID NO: 74, SEQ ID NO: 75, SEQ ID NO: 84 or SEQ ID NO: 85 to amplify part of the P450 monooxygenase cDNA from the cDNA library,

(c) optionally using one or more oligonucleotides designed on the basis of SEQ ID NO: 1, SEQ ID NO: 2, SEQ ID NO: 3, or SEQ ID NO: 4; SEQ ID NO: 9, SEQ ID NO: 10, SEQ ID NO: 11, or SEQ ID NO: 12; SEQ ID NO: 39 or SEQ ID NO: 40; SEQ ID NO: 54, SEQ ID NO: 55, SEQ ID NO: 56, SEQ ID NO: 70 or SEQ ID NO: 71; or SEQ ID NO: 74, SEQ ID NO: 75, SEQ ID NO: 84 or SEQ ID NO: 85 to amplify part of the P450 monooxygenase cDNA from the cDNA library in a nested PCR reaction,

(d) using the DNA obtained in steps (b) or (c) as a probe to screen the DNA library prepared from plant tissue expressing a P450 monooxygenase converting an aliphatic or aromatic amino acid or chain-elongated methionine homologue to the corresponding oxime, and

(e) identifying and purifying vector DNA comprising an open reading frame encoding a protein characterized by an amino acid sequence showing at least 40% or 50%, preferably 55%, or even more preferably 70% identity to the amino acid sequence resulting from the global alignment with SEQ ID NO: 1 or SEQ ID NO: 3 or both; SEQ ID NO: 9 or SEQ ID NO: 11 or both; SEQ ID NO: 39; SEQ ID NO: 54 or SEQ ID NO: 70 or both; or SEQ ID NO: 74 or SEQ ID NO: 84 or both,

(f) optionally further processing the purified DNA to achieve, for example, heterologous expression of the protein in a microorganism like Escherichia coli or Pichia pastoris for subsequent isolation of the monooxygenase, determination of its substrate specificity or generation of an antibody.

In process steps (b) and (c) the second oligonucleotide used for amplification is preferably an oligonucleotide complementary to a region within in the vector DNA used for preparing the cDNA library. However, a second oligonucleotide designed on the basis of the sequence of SEQ ID NO: 1, SEQ ID NO: 2, SEQ ID NO: 3, or SEQ ID NO: 4; SEQ ID NO: 9, SEQ ID NO: 10, SEQ ID NO: 11, or SEQ ID NO: 12; SEQ ID NO: 39 or SEQ ID NO: 40; SEQ ID NO: 54, SEQ ID NO: 55, SEQ ID NO: 56, SEQ ID NO: 70 or SEQ ID NO: 71; or SEQ ID NO: 74, SEQ ID NO: 75, SEQ ID NO: 84 or SEQ ID NO: 85 can also be used. cDNA clones coding for a P450 monooxygenase converting an aliphatic or aromatic amino acid or chain-elongated methionine homologue to the corresponding oxime or fragments of this clone may also be used on DNA chips alone or in combination with the cDNA clones encoding other proteins such as other proteins belonging to the CYP79 family of proteins or fragments of these clones. This provides an easy way to monitor the induction or repression of, for example, glucosinolate or cyanogenic glucoside synthesis in plants as a result of biotic and abiotic factors. Moreover, specific oligonucleotide sequences derived from the sequences of the present invention may be used as markers in marker assisted breeding programs or to identify such markers. Thus, the present invention allows to develop marker assisted breeding methods selecting desired traits using hybridization with one or more oligonucleotides, wherein the sequence of at least one of said oligonucleotides constitutes a component sequence of the DNA disclosed by the present invention. In a preferred embodiment said oligonucleotides consist of at least 15 and preferably at least 20 nucleotides and constitute components of a polymerase chain reaction assay.

Expressed as transgenes DNA encoding P450 monooxygenases according to the present invention is particularly useful to modify the biosynthesis of glucosinolates or cyanogenic glucosides in plants. When the gene encoding a cytochrome P450 enzyme converting an aliphatic or aromatic amino acid into the corresponding oxime is expressed in an acyanogenic plant together with a cytochrome P450 enzyme belonging to the CYP71 E family e.g. CYP71 El from sorghum or preferably the corresponding homolog from cassava and a UDP-glucose cyanohydrin glucosyltransferase, the transgenic plant obtained will be cyanogenic. The introduction of the gene encoding a cytochrome P450 enzyme converting an aliphatic or aromatic amino acid or chain-elongated methionine homologue into the corresponding oxime into a plant species producing glucosinolates can be used to alter the glucosinolate production in said plants as observed by an alteration of the overall level or the content of individual glucosinolates in the transgenic plants selected. If the aliphatic or aromatic amino acid or chain-elongated methionine homologue that is the substrate of the introduced cytochrome P450 enzyme was not previously recognized as a substrate for other cytochrome P450s in that particular plant species, then a new glucosinolate is introduced in the transformed plant. Likewise, the introduction of the gene encoding a cytochrome P450 enzyme converting an aliphatic or aromatic amino acid into the corresponding oxime into a cyanogenic plant can be used to modify the overall level and profile of the preexisting cyanogenic glucosides and to introduce one or more additional cyanogenic glucosides in the plant.

Proper selection of promoters to provide constitutive, inducible or tissue specific expression of the genes provides means to obtain transgenic plants with desired disease or herbivor responses. Likewise, the content of glucosinolates or cyanogenic glucosides in plants may be modified or reduced using anti-sense or ribozyme technology using the same genes. Thus, it is a further aspect of the present invention to provide transgenic plants comprising stably integrated into their genome DNA comprising at least part of an open reading frame of a P450 monooxygenase according to the present invention converting an aliphatic or aromatic amino acid or chain-elongated methionine homologue to the corresponding oxime. Such plants can be produced by a method comprising

(a) introducing into a plant cell or tissue which can be regenerated to a complete plant, DNA comprising at least part of an open reading frame of a P450 monooxygenase according to the present invention converting an aliphatic or aromatic amino acid or chain-elongated methionine homologue to the corresponding oxime; and

(b) selecting transgenic plants.

Preferably said method either results in plants transgenically expressing said P450 monooxygenase or in plants with reduced expression of an endogenous P450 monooxygenase or in plants with reduced production of glucosinolates or cyanogenic glucosides.

### EXAMPLES

### Example 1

PCR Amplification of Cassava CYP79 Probes and Library Screening

Based on the assumption that the P450 enzyme catalyzing conversion of L-valine to the corresponding oxime belongs to the CYP79 family, degenerate primers are designed towards areas showing sequence conservation in CYP79A1 (sorghum), CYP79B1 (Sinapis alba) and CYP79B2 (Arabidopsis thaliana). Domains putatively involved in substrate recognition are excluded for primer design, because none of the known CYP79s utilizes valine or isoleucine as a substrate.

First round PCR amplification reactions in a total volume of 20 μl are carried out in 10 mM Tris-HCl pH 9, 50 mM KCl, 1.5 mM MgCl₂ using 0.5 U Taq DNA polymerase (Pharmacia, Sweden), 200 μM dATP, 200 μM dCTP, 200 μM dGTP, 200 μM dTTP, 500 nM of each of the primers 5′-GCGGAATTCARGGIAAYCCIYTICT-3′ (SEQ ID NO: 5) and 5′-CGCGGATCCGGDATRTCIGAYTCYTG-3′ (SEQ ID NO: 6), wherein I represents inosine, and 10 ng of plasmid DNA template. The plasmid DNA template is prepared from a unidirectional plasmid cDNA library in pcDNA2.1 (Invitrogen, The Netherlands) made from immature folded leaves and petioles of shoot tips of cassava plants. Thermal cycling parameters are 95° C. for 2 min, 3 cycles of (95° C. for 5 s, 40° C. for 30 s, and 72° C. for 45 seconds; 32 cycles of 95° C. for 5 s, 50° C. for 5 s, and 72° C. for 45 s; and a final 72° C. elongation for 5 min. A of the expected size of 210 bp is stabbed out with a Pasteur pipette and used for second round PCR amplifications in 50 μl of the same reaction mixture as above using 95° C. for 2 min, 20 cycles of 95° C. for 5 s, 50° C. for 5 s, and 72° C. for 45 s; and a final 72° C. elongation for 5 min. The product is sequenced with the Thermo Sequenase radiolabeled terminator cycle sequencing kit (Amersham, Sweden) and α-³³P-ddNTP (Amersham, Sweden) according to the manufacturer. The gene specific fragment is labeled with digoxigenin-11-dUTP (Boehringer Mannheim, Germany) by PCR amplification and used as probe to screen the cassava cDNA library using the DIG system (Boehringer Mannheim, Germany). The probe is hybridized over night at 68° C. in 5×SSC, 0.1% N-lauroylsarcosine, 0.02% SDS, 1% blocking reagent (Boehringer Mannheim, Germany). Prior to detection, filters are washed with 0.1×SSC, 0.1% SDS at 65° C.

### Example 2

CYP79D1 and CYP79D2, Sequencing and Southern Blot Analysis

Using the probe obtained according to example 1 two equally abundant full-length clones are isolated from the cassava cDNA library. The clones have open reading frames encoding P450s of 61.2 and 61.3 kDa. These P450s are assigned CYP79D1 and CYP79D2 as the first two members of a new CYP79D subfamily. Sequencing is performed using the Thermo Sequenase Fluorescent-labeled Primer cycle sequencing kit (7-deaza dGTP) (Amersham, Sweden) and an ALF-Express sequenator (Pharmacia, Sweden). Sequence computer analysis is performed using the programs from the GCG Wisconsin Sequence Analysis Package. The two cassava P450s are 85% identical and both share 54% identity to CYP79A1. P450s showing more than 40% but less than 55% sequence identity at the amino acid level are grouped in the same family but in different subfamilies. The heme-binding motif in CYP79D1 and CYP79D2 is TFSTGRRGCVA (residues 470-480 of CYP79D1) and contains three amino acid substitutions compared to the consensus sequence PFGXGRRXCXG for A-type P450s (Durst et al, Drug Metabol Drug Interact 12: 189-206,1995). The substitutions underlined are also found in CYP79A1 whereas the initial T in the CYP79D1 and CYP79D2 heme-binding motif is an S in CYP79A1, CYP79B1 and CYP79B2. Thus, the previously proposed existence of a heme binding sequence domain unique to the CYP79 family is contradicted. The other unique sequence domain PERH (residues 450-453 of CYP79D1), where H has been proposed to be specific for the CYP79 family is also found in CYP79D1 and CYP79D2.

To determine the copy number of CYP79D1 and CYP79D2 a Southern Blot on genomic DNA from the cassava cultivar MCol22 is performed. Genomic DNA is purified from leaves of cassava cultivar Mcol22 as described by Chen et al in: The Maize Handbook (Freeling et al eds), Springer Verlag, N.Y., 1994. The DNA is further purified on Genomic-tip 100/G (Qiagen, Germany), digested with restriction enzymes and electrophoresed (10 μg DNA/lane) on a 0.6% agarose gel in 1× TAE. The gel is blotted to a nylon membrane (Boehringer-Mannheim, Germany) and hybridized at 68° C. with the radiolabeled CYP79D1 or CYP79D2 clone. After hybridization, the membrane is washed twice in 2×SSC, 0.1% SDS at room temperature and twice in 0.1×SSC, 0.1% SDS at 68° C. Radiolabeled bands are visualized using a Storm 840 phosphor imager (Molecular Dynamics, CA, USA). The probes for Southern hybridization are labeled with a Random Primed DNA Labeling Kit (Boehringer-Mannheim, Germany) using α-³²P-dCTP. The two probes hybridize to different bands on the Southern blot demonstrating that both genes are present in the MCol22 genome. The high similarity between the genes results in weak cross hybridization. Low stringency washing (0.5×SSC, 0.1% SDS at 55° C.) does not reveal additional copies of the CYP79D genes.

### Example 3

### Recombinant Expression in P. pastoris

Generation of recombinant P. pastoris containing CYP79D1 or CYP79D2 is achieved using the vector pPICZc (Invitrogen, The Netherlands). This vector contains the methanol inducible AOX1 promoter for control of gene expression and encodes resistance against zeocin and is used to achieve intracellular expression of CYP79D1 or CYP79D2 in P. pastoris wild type strain X-33 (Invitrogen, The Netherlands). E. coli strain TOP10F′ is used for transformation and propagation of recombinant plasmids. An XhoI site is introduced immediately downstream of the CYP79D1 stop codon by PCR. The PCR product is restricted with XhoI and with BsmBI. The latter enzyme cuts 18 bp downstream of the start ATG codon. pPICZc is restricted with BstBI and XhoI. The vector and PCR product are ligated together using an adapter made from the following annealed oligos:

(SEQ ID NO: 7; sense direction)

5′-CGAAACGATGGCTATGAACGTCTCT-3′ and

(SEQ ID NO: 8)

5′-TGGTAGAGACGTTCATAGCCATCGTTT-3′.

The adapter on the one hand reestablishes the first 18 bp of CYP79D1 (start codon underlined) introducing two silent mutations, and on the other hand a short vector sequence removed by BstBI restriction, thereby positioning the CYP79D1 start codon exactly as the start codon of the highly expressed AOXI gene product. CYP79D2 is cloned into pPICZc in a similar manner using the same adapter because the coding sequences of CYP79D1 and CYP79D2 genes are identical for the first 24 bp. Transformation of P. pastoris is achieved by electroporation according to the Invitrogen manual (EasySelect Pichia expression Kit Version A, Invitrogen, The Netherlands). The presence of CYP79D1 or CYP79D2 in zeocin resistant colonies is confirmed by PCR on the P. pastoris colonies. Single colonies of P. pastoris are grown (28° C., 220 rpm) for approximately 22 h in 25 ml BMGY (1% yeast extract, 2% peptone, 0.1 M KPᵢ pH 6.0, 1.34% yeast nitrogen base, 4×10⁻⁵% biotin, 1% glycerol, 100, μg/ml zeocin). Cells are harvested (1500 g, 10 min, RT) and inoculated in a 2 l baffled flask to OD₆₀₀ of 0.5 in 300 ml of inducing medium, i.e. BMGY with 1% methanol instead of glycerol. The cultures are grown (28° C., 300 rpm) for 28 h with addition of methanol to 0.5% after 26 h. Cells are pelleted (3000 g, 10 min, 4° C.) and washed once in buffer A (50 mM KPᵢ pH 7.9, 1 mM EDTA, 5% glycerol, 2 mM DTT, 1 mM phenylmethylsulfonyl fluoride) before being resuspended to OD₆₀₀ of 130 in buffer A. An equal volume of acid-washed glass beads is added and the cells are broken by vortexing (8×30 s, 4° C. with intermediate cooling on ice). The lysate is centrifuged at 12000 g (10 min, 4° C.) to remove cell debris and the resulting supernatant recentrifuged at 165000 g (1 h, 4° C.) to recover a microsomal pellet. Microsomes are resuspended in buffer A, stored at −80° C. and thawed on ice immediately before use. CYP79D1 and CYP79D2 are functionally expressed in P. pastoris as evidenced by the ability of recombinant yeast cells to convert L-valine to the corresponding. No conversion took place using P. pastoris cells transformed with the vector only. The metabolic activity is measured in intact cells demonstrating that the endogenous P. pastoris reductase system is able to support electron donation to these plant P450s. SDS-PAGE of microsomes prepared from cells actively converting L-valine to val-oxime shows the presence of an additional polypeptide band migrating corresponding to a molecular mass of 62 kDa as expected from the CYP79D1 cDNA clone. With regard to CYP79D1 activity in intact P. pastoris cells the best results were obtained using growth in rich media and induction at OD 0.5 for 24-30 h. 15-30 nmol of microsomal CYP79D1 per liter culture are produced. The yield of microsomal CYP79D1 after 90 h of induction is 50% of that obtained after 24 h.

### Example 4

Purification of Recombinant CYP79D1

All steps are carried out at 4° C. unless otherwise stated. CYP79D1 containing fractions are identified by carbon monoxide difference spectroscopy, SDS-PAGE and activity measurements. Recombinant CYP79D1 is isolated using P. pastoris microsomes as the starting material and TX-114 phase partitioning (Bordier, J Biol Chem 256: 1604-1607, 1981; Werck-Reichhart et al, Anal Biochem 197: 125-131, 1991) as the first purification step. The phase partitioning mixture contains microsomal protein (4 mg/ml), 50 mM KPᵢ pH 7.9, 1 mM DTT, 30% glycerol and 1% TX-114. After stirring (4° C., 30 min) phase separation is achieved by temperature shift and centrifugation (22° C., 24500 g, 25 min, brake off). The reddish TX-114 rich upper phase is collected and the TX-114 poor lower phase is re-extracted with 1% TX-114. The rich phases are combined and diluted in buffer B (10 mM KPᵢ pH 7.9, 2 mM DTT) to a TX-114 concentration less than 0.2%. The TX-114 rich phase is applied with a flow rate of 25 ml/h to a 2.6×2.8 cm column of DEAE Sepharose FF (Pharmacia, Sweden) connected in series to a 1.6×3 cm column of Reactive Red 120 agarose (Sigma, MO, USA). Both columns are equilibrated in buffer C (10 mM KPᵢ pH 7.9, 10% glycerol, 0.2% TX-114, 2 mM DTT). After sample application, the columns are washed thoroughly (over night) in buffer C. CYP79D1 does not bind to the ion exchange column under these conditions and is recovered from the Reactive Red 120 agarose by gradient elution (50 ml, 0 to 1.5 M KCl in buffer C). Fractions containing fairly pure CYP79D1 are combined, dialyzed over night against buffer C and applied to a 1.6×2.2 cm column of Reactive Yellow 3A agarose (Sigma, MO, USA) equilibrated in buffer C. The column is washed using buffer C and CYP79D1 obtained by gradient elution (50 ml, 0 to 1.5 M KCl in buffer C). The fractions containing homogenous CYP79D1 are combined and dialyzed for 2 h against buffer D (10 mM KPᵢ pH 7.9, 10% glycerol, 50 mM NaCl, 2 mM DTT) to reduce salt and detergent. CYP79D1 is stored in aliquots at −80° C. SDS-PAGE is performed using high Tris linear 8-25% gradient gels (Fling et al, Anal Biochem 155: 83-88, 1986). Total P450 is quantified by carbon monoxide difference spectroscopy on a SLM Aminco DW-2000 TM spectrophotometer (Spectronic Instruments, NY, USA) using a molar extinction coefficient of 91 mM⁻¹ cm⁻¹ for the adduct between reduced P450 and carbon monoxide (Omura et al, J. Biol. Chem. 249: 5019-5026, 1964). Substrate-binding spectra are recorded according to the method of Jefcoate (Jefcote, Methods Enzymol 27: 258-279, 1978) in 50 mM KPᵢ pH 7.9, 50 mM NaCl.

Purified CYP79D1 migrates with a molecular mass of 62 kDa. The overall yield of the isolation procedure is 17%, i.e. 1 nmol CYP79D1 is obtained from 260 ml of culture. It consistently produces an absorption maximum at 448 nm when subjected to CO difference spectroscopy. No maximum is observed at 420 nm using either isolated or crude fractions. This demonstrates that CYP79D1 is a fairly stable protein. Yeast cytochromes may interfere with the spectroscopy of crude extracts and hide a minor 420 nm peak and P. pastoris cytochrome oxidase had previously been reported to prevent P450 spectroscopy. In the present study, the expression level of CYP79D1 is high and the CO difference spectrum produced by cytochrome oxidase (maximum at 430 nm, minimum at 445) is visible as a shoulder on the 450 nm peak. The P. pastoris cytochrome oxidase binds to the DEAE column and accordingly is removed during P450 isolation. Upon culturing P. pastoris for extended periods (90 h), the content of cytochrome oxidase decreases permitting detection of lower amounts of P450 in microsomes. Finally, interfering cytochrome oxidase can be removed from P450 by TX-114 phase partitioning performed in borate buffer. Upon phase partitioning in borate, the P450s partition to the TX-114 poor phase, whereas P. pastoris cytochrome oxidase partitiones to the rich phase. Purified CYP79D1 forms a type I substrate binding spectrum in the presence of L-valine corresponding to a 44% shift from low spin to high spin state upon substrate binding.

### Example 5

Determination of the Catalytic Activity

Isolated, recombinant CYP79D1 is reconstituted and its catalytic activity determined in vitro using reaction mixtures with a total volume of 30 μl containing 2.5 pmol CYP79D1, 0.05 U NADPH P450-oxidoreductase (Benveniste et al, Biochem J 235: 365-373, 1986), 10.6 mM L-α-dioleyl phosphatidylcholine, 0.35 μCi [U-¹⁴C]-L-amino acid (L-Val, L-Ile, L-Leu, L-Tyr or L-Phe; Amersham, Sweden), 1 mM NADPH, 0.1 M NaCl and 20 mM KPᵢ pH 7.9. In assays containing ¹⁴C-L-valine or ¹⁴C-L-isoleucine, different amounts of unlabeled L- and D-amino acids (0-6 mM) are added. After incubation for 10 minutes at 30° C. the products formed are extracted into 60 μl ethyl acetate and separated on TLC sheets (Merck Kieselgel 60F₂₅₄) using n-pentane/diethyl ether (50:50, v/v) or toluene/ethyl acetate (5:1, v/v) as eluents for aliphatic compounds and aromatic compounds, respectively. ¹⁴C-labeled oximes are visualized and quantified using a STORM 840 phosphor imager (Molecular Dynamics, CA, USA). The activity of CYP79D1 is additionally measured in the presence of the inhibitors tetcyclasis, ABT and DPI under the same conditions as described above. For in vivo activity assays 200 μl P. pastoris cells are pelleted and resuspended in 100 μl 50 mM Tricine pH 7.9 and 0.35 μCi [U-¹⁴C]-L-valine or L-isoleucine. After incubation for 30 minutes at 30° C. the cells are extracted with ethyl acetate and the products formed are analyzed as above.

CYP79D1 is reconstituted with sorghum NADPH-P450 oxidoreductase in the presence of high amounts of the lipid L-α-dioleyl phosphatidylcholine and 100 mM NaCl. The five protein amino acids used in plants as precursors for cyanogenic glucoside synthesis are tested as substrates for CYP79D1. The corresponding oximes are formed from L-valine or L-isoleucine. Using L-leucine, L-phenylalanine or L-tyrosine as substrates no metabolism is evident at a detection level equal to 0.8% of the metabolism observed with L-valine. The observed substrate specificity corresponds with the in vivo presence of only L-valine and L-isoleucine derived cyanogenic glucosides in cassava. To examine the effect of inhibitors on isolated CYP79D1, reconstitutions are performed in the presence of tetcyclasis, ABT and DPI using the same conditions as for cassava microsomes. The same pattern as in cassava microsomes is observed using isolated CYP79D1. CYP79D1 is inhibited by tetcyclasis, but not by ABT. Similar to the situation in cassava microsomes, DPI completely inhibits the val-oxime formation by inhibiting the NADPH-P450 oxidoreductase. When cassava microsomes are used, cyanide is produced with L-valine and L-isoleucine as substrates, whereas no metabolism is observed using D-valine and D-isoleucine. A higher conversion rate is observed using L-valine compared to L-isoleucine similar to the data obtained using microsomes prepared from etiolated cassava seedlings. Isolated CYP79D1 produces ¹⁴C-labeled val-oxime from ¹⁴C-L-valine. When the specific activity of the ¹⁴C-L-valine substrate is reduced 120 times by addition of unlabeled L-valine, a corresponding reduction of the amount of ¹⁴C-labeled oxime formed is observed. However, addition of unlabeled D-valine to the incubation mixture does not result in a corresponding reduction in the amount of ⁴C-labeled oxime formed. Thus, neither the cassava microsomes nor isolated CYP79D1 metabolize D-valine. The lack of competition of D-valine with L-valine indicates that D-valine does not bind with high affinity to the active site of CYP79D1. Similar results are obtained with ¹⁴C-L-isoleucine, L-isoleucine and D-isoleucine . Under saturating substrate conditions CYP79D1 has a higher conversion rate using L-valine as substrate. The conversion rate of L-isoleucine is approximately 60% of that observed for L-valine. This is consistent with higher accumulation of linamarin compared to lotaustralin in vivo in cassava (4).

### Example 6

N-Terminal Sequencing of CYP79D1

Isolated recombinant CYP79D1 is subjected to SDS-PAGE and the protein transferred to ProBlott membranes (Applied Biosystems, CA, USA) as described in Kahn et al, J. Biol. Chem 271: 32944-32950, 1996. The Coomassie Brilliant Blue-stained protein band is excised from the membrane and subjected to sequencing on an Applied Biosystems model 470A sequenator equipped with an on-line model 120A phenylthiohydantoin amino acid analyzer. Asn glycosylation is detected as the lack of an Asn signal in the predicted Edman degradation cycle. The fractions that produce CO spectra and contain CYP79D1 activity always produce two distinct closely migrating polypeptide bands upon SDS-PAGE. N-terminal amino acid sequencing identifies both bands as derived from CYP79D1. The initial methionine is removed by the yeast processing system. Sequencing of the first 15 residues of the upper band demonstrates glycosylation of both asparagines present, whereas the lower band only is glycosylated at the first asparagine. The different glycosylation pattern explains the presence of two bands. Glycosylation at the N-terminal part of CYP79D1 is in agreement with the localization of the N-terminal in the lumen of the endoplasmatic reticulum accessible for the glycosylation machinery. It is unknown, whether native CYP79D1 is glycosylated in cassava. However, CYP79A1 purified from sorghum seedlings is not glycosylated as documented by amino acid sequencing of the N-terminal fragment (15) and only few reports exist of microsomal P450 glycosylation. The observed glycosylation of recombinant CYP79D1 upon expression in P. pastoris is thought to reflect expression in a yeast system.

### Example 7

Primers Used in Examples 8 and 9

| | | |
|---|---|---|
| Primer Designation | Nucleotide sequence^{a} | SEQ ID NO: |

| | | |
|---|---|---|
| 1F^{b} | GCGGAATTCGAYAAYCCIWSIAAYGC | 13 |
| 1R^{b} | GCGGATCCGCIACRTGIGGIAHRTTRAA | 14 |
| 2F | GCGGAATTCWSIAAYGCIRTIGARTGG | 15 |
| 2R | GCGGATCCRTTRAAIIINGCIACIGGRTG | 16 |
| 3F | GCGGAATTCCACACAGGAAACAGCTATGAC | 17 |
| 3R^{e} | GCGGATCCAGACGAGTAGCGAGTCACAAC | 18 |
| 4R#1^{f} | GCGGATCCAAGAGGAACAGTACT | 19 |
| 4R#2^{f} | GCGGATCCAAGAGGAACAATGTG | 20 |
| 5F#1^{f} | GCGAATGCATTGCTCCCACTAGCC | 21 |
| 5R#1^{f} | GCGATGGTTATGAGTTCCATTTTG | 22 |
| 6F#1(na) | GCGCATATGGAACTAATAACAATTCTT | 23 |
| 6R | GCGAAGCTTATTAGAAGCTCTGGAGCAG | 24 |
| 6F#1(Δ(1-31)_{17α(8aa)}) | GCGCATATGGCTCTGTTATTAGCAGTTTTTTTCC- | 25 |
| | TCTTCCTCTTCAAACAA | |
| 6F#1(Δ(1-52)_{2E1(10aa)}) | GCGCATATGGCTCGTCAAGTTCATTCTTCTTGG- | 26 |
| | AATTTACCACCAGGCCCC | |

^{a}The sequence is shown from 5′ end to 3′ end.
^{b}F: forward primer, R: reverse primer.
^{e}Covers a sequence that is identical in the two clones #1 and #2.
^{f}Covers a sequence that is specific for either of the two clones #1 and #2.

| | | | |
|---|---|---|---|
| Primer Designation | Restriction Site | Amino acids encoded | SEQ ID NO: |

| | | | |
|---|---|---|---|
| 1F^{b} | EcoRI | DNPSNA^{c} | 27 |
| 1R^{b} | BamHI | FNV/LPHVA^{c} | 28 |
| 2F | EcoRI | SNAVEW^{c} | 29 |
| 2R | BamHI | HPVAXFN^{c} | 30 |
| 3F | EcoRI | ^{d} | |
| 3R^{e} | BamHI | VVTRYSS | 31 |
| 4R#1^{f} | BamHI | TVLFLL | 32 |
| 4R#2^{f} | BamHI | ATLFLL | 33 |
| 5F#1^{f} | | ^{g} | 35 |
| 5R#1^{f} | | MELITI | 34 |
| 6F#1(na) | Ndel | MELITIL | |
| 6R | HindIII | LLQSF*^{h} | 36 |
| 6F#1(Δ(1-31)_{l7α(8aa)}) | Ndel | MALLLAVFFLFLFKQ | 37 |
| 6F#1(Δ(1-52)_{2E1(10aa)}) | Ndel | MARQVHSSWNLPPGP | 38 |

^{b}F: forward primer, R: reverse primer.
^{c}Amino acid consensus sequence used for primer design.
^{d}A specific primer for pcDNA2.1 placed just upstream the insertion site of the 5′ end of the cDNA library.
^{e}Covers a sequence that is identical in the two clones #1 and #2.
^{f}Covers a sequence that is specific for either of the two clones #1 and #2.
^{g}A specific primer for the 5′UTR in #1.
^{h}The star indicates a stop codon.

### Example 8

cDNA Cloning of Triglochin maritima CYP79 Genes

PCR approach to generate cDNA fragments of a CYP79 homologue in T. maritima A unidirectional plasmid cDNA library is made by In Vitrogen (Carlsbad, Calif.) from flowers and fruits (schizocarp) of T. maritima, using the expression vector pcDNA2.1 which contains the lacZ promoter. Plant material is collected at Aflandshage on Southern Amager, at the coast of Øresund, frozen directly in liquid N₂ and stored at −80° C. Degenerate PCR primers are designed based on conserved amino acid sequences in CYP79A1 derived from S. bicolor—GenEMBL U32624, CYP79B1 from Sinapis alba—GenEMBL AF069494, CYP79B2 from Arabidopsis thaliana—GenEMBL, and a PCR fragment of CYP79D1 from Manihot esculenta—GenEMBL AF140613. Two rounds of PCR amplification reactions in a total volume of 50 μl are carried out using 100 pmol of each primer, 5% dimethyl sulfoxide, 200 μM dNTPs and 2.5 units Taq DNA polymerase in PCR buffer (50 mM KCl, 10 mM Tris-HCl pH 8.8, 1.5 mM MgCl₂, 0.1% Triton X-100). Thermal cycling parameters are 2 min at 95° C., 30×(5 sec at 95° C., 30 sec at 45° C., 45 sec at 72° C.) and finally 5 min at 72° C. The first PCR reaction is performed using primers 1F and 1R (Example 7) on 100 ng template DNA prepared from the cDNA library or genomic DNA prepared using the Nucleon Phytopure Plant DNA Extraction Kit (Amersham). The PCR products are purified using QIAquick PCR Purification Kit (Qiagen), eluted in 30 μl 10 mM Tris-HCl pH 8.5, and used as template (1 μl) for the second round of PCR reactions carried out using PCR fragments derived from both cDNA and genomic DNA and using the two degenerate primers 2F and 2R (Example 7). An aliquot (5 μl) of the PCR reaction is applied to a 1.5% agarose/TBE gel and a band of the expected size of about 200 bp is observed using both cDNA and genomic DNA as template. The rest of the PCR reaction is purified using QIAquick PCR Purification Kit and eluted in 30 μl 10 mM Tris-HCl pH 8.5. The purified PCR fragments (5 μl) are digested with EcoRI and BamHI, excised from a 1.5% agarose/TBE gel, purified using QIAEX II Agarose Gel Extraction kit (Qiagen) and ligated into an EcoRI- and BamHI-digested pBluescript II SK vector (Stratagene). Seven clones derived from the cDNA library and three clones derived from genomic DNA are sequenced (ALF Express, Pharmacia) using the Thermo Sequenase Fluorescent-labeled Primer cycle sequencing kit with 7-deaza dGTP (Amersham). Sequence analyses is performed using programs in the GCG Wisconsin Sequence Analysis package.

Screening of a Plasmid cDNA Library Made From Flowers and Fruits of T. maritima

Both cDNA and genomic DNA produce an identical PCR fragment with high sequence resemblance to the other known CYP79 sequences. The cloned PCR fragment is used as template to generate a 350 bp digoxigenin-11-dUTP-labeled probe (TRI1) by PCR, using the commercially available T3 and T7 primers. The labeled probe is used to screen 660.000 colonies of the pcDNA2.1 cDNA library. Hybridizations are carried out overnight at 68° C. in 5×SSC (0.75 M NaCl, 75 mM sodium citrate pH 7.0), 0.1% N-lauroylsarcosine, 0.02% sodium dodecyl sulfate and 1% Blocking Reagent (Boehringer Mannheim). Membranes are washed twice under high stringency conditions (65° C., 0.1×SSC, 0.1% sodium dodecyl sulfate), incubated with Anti-Digoxigenin-AP and developed using 5-bromo-4-chloro-3-indolylphosphate and nitroblue tetrazolium according to Boehringer Mannheims instructions. Positive colonies are rescreened under the same conditions, and single positive colonies are sequenced and analyzed.

PCR Approach to Design 5′ End Probes to Screen for Full Length Clones

The library screens described above result in two very similar partial clones designated #1 and #2, particularly differing in their N-terminal sequence. To isolate the corresponding full length clones from the pcDNA2.1 library, two consecutive PCR reactions are performed using the same PCR conditions as above, with the exception that the annealing temperature is set at 55° C. The first PCR reaction is performed with primers 3F and 3R (Example 7) using 100 ng cDNA library template. The purified PCR products (QIAquick PCR Purification Kit) from the first PCR reaction are used as template (1 μl) for a second round of PCR reactions using primer 4R#1 or 4R#2 against primer 3F (Example 7). The PCR fragments from the second round are separated on a 2% agarose/TBE gel and the slowest migrating bands are excised from the gel, purified (QIAEX II Agarose Gel Extraction kit), digested with EcoRI and BamHI, cloned in pBluescript II SK and sequenced. Using primer 4R#1 together with primer 3F (Example 7) in the second round PCR, a PCR fragment with a putative start methionine 26 amino acids downstream the EcoRI cloning site is obtained. The PCR reaction with primers 4R#2 and 3F (Example 7) produces a PCR fragment of exactly the same length as the partial cDNA clone already isolated using the TRI1 probe. As a consequence, the PCR fragment cloned with 4R#1 and 3R is used as a template to generate a digoxigenin-11-dUTP labeled probe (TRI2) using primers 5F#1 and 5R#1 (Example 7). Using the same conditions as above, TRI2 partly covering the 5′ untranslated region (UTR) and 5′ end of the open reading frame of clone #1 is used to screen the pcDNA2.1 library together with the TRI1 probe. The first lifts are hybridized with TRI2 and the second with TRI1. Two individual cDNA clones with exactly the same length as the PCR fragment are isolated after screening 1.000.000 colonies.

Results

Based on a sequence alignment of CYP79A1 and putative N-hydroxylases belonging to the CYP79 family, four degenerate oligonucleotide primers covering two CYP79 specific regions are designed (1F, 2F, 1R, 2R described in Example 7) and used in nested PCR reactions with genomic DNA as well as cDNA made from flowers and fruits of Triglochin maritima as templates. A PCR fragment of the expected size, i.e. approximately 200 bp, and showing 62 to 70% identity to CYP79 sequences at the amino acid level is amplified from both templates, cloned and further used to screen the cDNA library. Two cDNA clones, denoted #1 and #2, are isolated and verified by sequence comparison to share high sequence identity to the CYP79 family. Using clone specific PCR primers, a full-length clone corresponding to #1 is isolated. The open reading frame encodes a protein with a molecular mass of 60.8 kDa. A comparison of the full-length sequence of clone #1 with that of clone #2 reveals that clone #2 is 6 bp shorter at the 5′ end but contains a methionine codon not found in clone #1 at a position corresponding to amino acid residue 26 specified by clone #1. The sequence surrounding this methionine codon does not fit the general context sequence for a start codon in a monocotyledonous plant. Most likely, clone #2 thus lacks 6 bp to be full-length.

The cytochrome P450s encoded by clones #1 and #2 show 44 to 48% identity to already known members of the CYP79 family (see Table below) and accordingly are identified as the first two members of the new subfamily CYP79E and assigned CYP79E1 (SEQ ID NO: 9) and CYP79E2 (SEQ ID NO: 11). The sequence identity between CYP79E1 and CYP79E2 is 94%.

TABLE
% Identity and similarity between six members of the CYP79 family

Similarity

| Identity | CYP79E1 | CYP79E2 | CYP79A1 | CYP79B1 | _CYP79B2 | CYP79D1 |
|---|---|---|---|---|---|---|
| CYP79E1 | | 95.2 | 61.7 | 58.1 | 58.9 | 60.0 |
| CYP79E2 | 94.1 | | 61.5 | 57.6 | 58.5 | 59.2 |
| CYP79A1 | 48.8 | 48.8 | | 65.5 | 67.1 | 65.8 |
| CYP79B1 | 44.9 | 44.9 | 51.3 | | 92.3 | 65.1 |
| CYP79B2 | 44.5 | 44.6 | 52.6 | 89.3 | | 67.3 |
| CYP79D1 | 46.4 | 46.5 | 51.5 | 49.1 | 50.7 | |

### Example 9

Recombinant Expression in E. coli

Expression Constructs

The expression vector pSP19g10L is used for expression of CYP79E1 and CYP79E2 constructs in E. coli. This expression vector contains the lacZ promoter fused with the short leader sequence of gene 10 from T7 bacteriophage (g10 L) and has been shown effective for heterologous protein expression in E. coli (Olins et al, Methods Enzymol. 185: 115-119, 1990). In case of cytochrome P450s, increased expression levels have been obtained by modifying the 5′ end of the open reading frame to increase the content of A's and T's (Stormo et al, Nucleic Acids Res. 10: 2971-2996, 1982; Schauder et al, Gene 78: 59-72, 1989; Barnes et al, Proc. Natl. Acad. Sci. USA 88: 5597-5601, 1991) and by replacement of a number of codons at the 5′ end with codons specifying the N-terminal sequence of bovine P45017α (Barnes et al, Proc. Natl. Acad. Sci. USA 88: 5597-5601, 1991) or human P4502E1 or 2D6 (Gillam et al, Arch. Biochem. Biophys. 312: 59-66, 1994; Gillam et al, Arch. Biochem. Biophys. 319: 540-550, 1995. To take advantage of this knowledge, a number of different constructs are made.

Three different constructs of clone #1 are generated with PCR, using Pwo polymerase (Boehringer Mannheim) to introduce a NdeI restriction site at the start codon and a HindIII restriction site immediately after the stop codon. A full length construct (CYP79E1ₙₐ) encoding native CYP79E1 with silent mutations introduced at codons 3 and 5 to increase the AT content is synthesized using primers 6F#1(na) and 6R#1 (Example 7). Two truncated constructs are made using primers 6F#1(Δ(1-31)_{17α(8aa)}) and 6R#1 or primers 6F#1(Δ(1-52)_{2E1(10aa)}) and 6R#1 (Example 7). Construct CYP79E1Δ(1-31)_{17α(8aa)} encodes a truncated form of CYP79E1 in which 31 codons of the native 5′ sequence are replaced by 8 AT-enriched codons of P45017α (Halkier et al, Arch. Biochem. Biophys. 322: 369-377, 1995; Barnes et al, Proc. Natl. Acad. Sci. USA 88: 5597-5601, 1991); in construct CYP79E1Δ(1-52)_{2E1(10aa)} the first 52 codons of the native 5′sequence are replaced by 10 AT-enriched codons of P4502E1 and silent mutations are introduced in codons 53 and 55.The PCR fragments are digested with NdeI and HindIII and ligated into NdeI- and HindIII-digested pSP19g10L expression vector (Barnes, Methods Enzymol. 272: 3-14, 1996). The unique restriction sites NcoI and PmlI are used to replace the middle part of the PCR clones (1045 bp) with the analogous fragment from the cDNA clone. The remaining portions of the constructs deriving from PCR, are sequenced to exclude PCR errors.

Because the CYP79E2 clone is isolated in frame with the first 24 codons of the lacZ gene in the vector pcDNA2.1, this clone is tested as a fourth expression construct designated CYP79E2_{lacZ(24aa)}. For comparison, an equivalent fifth construct CYP79E1Δ(1-2)_{lacZ(24aa)} is also prepared.

All constructs contain the original stop sequence TAAT found in most highly expressed E. coli genes. All constructs using the vector pSP19g10L have their 3′UTR removed, because inclusion of the 3′UTR has been reported to prevent or reduce expression of some genes. In constructs based on pcDNA2.1, the 3′UTR is retained.

Expression in E. coli

All expression constructs are transformed into the E. coli strains JM109 (Stratagene) and XL-1 blue (Stratagene). In all cases, the JM109 strain turns out to be most efficient.

CYP79E1 and CYP79E2 contain 19 and 17 AGA or AGG arginine codons which are rare in E. coli genes. A strong positive correlation between the occurrence of codons and tRNA content has been established. Accordingly, the native and Δ(1-52)_{2E1(10aa)} constructs of clone #1 as well as the construct of clone #2 are co-transformed with pSBET (Schenk et al, BioTechniques 19: 196-200, 1995) encoding a tRNA gene for rare arginine codons, into JM109. Single colonies are grown overnight in LB medium (50 μg/ml ampicillin, 37° C., 225 rpm) and used to inoculate 100×volume of modified TB medium (50 μg/ml ampicillin, 1 mM thiamine, 75 μg/ml δ-amino-levulinic acid, 1 mM isopropyl β-D-thiogalactopyranoside (IPTG)) for growth at 28° C. and 125 rpm for 48 hours.

Measurements of Expression Levels and Biosynthetic Activities

Expression levels of the different constructs are determined by CO difference spectroscopy and quantified using an extinction coefficient ε₄₅₀₋₄₉₀ of 91 mM⁻¹cm⁻¹ (Omura et al, J. Biol. Chem. 239: 2370-2378, 1964). Spectra are made from 100 μl or 500 μl whole E. coli cells or using the rich phases from Triton X-114 phase partitioning solubilized in 50 mM KH₂PO₄/K₂HPO₄ pH 7.5, 2 mM EDTA, 20% glycerol, 0.2% Triton X-100 (total volume: 1 ml). E. coli cells for in vivo studies are prepared by centrifugation (2 min and 30 sec at 7000 g) of 1 ml cell culture and resuspension in 100 μl 50 mM tricine pH 7.9, 1 mM phenylmethylsulfonyl fluoride. For in vitro studies, spheroblasts are made from E. coli (JM109) cells expressing native or Δ(1-52)_{2E1(10aa)} constructs of clone #1 or the construct of clone #2, followed by temperature-induced phase partitioning (0.6% Triton X-114, 30% glycerol) as previously described (Halkier et al, Arch. Biochem. Biophys. 322: 369-377, 1995). Measurements of in vivo catalytic activity are carried out by administration of [U-¹⁴C]tyrosine (0.35 μCi, 7.39 μM), p-hydroxyphenylacetaldoxime (0 or 0.1 mM) or p-hydroxyphenylacetonitrile (0 or 0.1 mM) to resuspended 100 μl of E. coli cells. In vitro activities are measured in reconstitution experiments using the rich phase from phase partitioning. A standard reaction mixture (total volume: 50 μl) contains 5 μl rich phase, 0.375 U of S. bicolor NADPH-cytochrome P450 oxidoreductase, 5 μl L-α-dilauroyl phosphatidylcholine (DLPC), 0.6 mM NADPH and 14 mM KH₂PO₄/K₂HPO₄ pH 7.9. The following substrates are tested: L-[U-¹⁴C]tyrosine (0.20 μCi, 9.04 μM), L-[U-¹⁴C]phenylalanine (0.20 μCi, 8.8 μM) and L-3,4-dihydroxyphenyl[3-¹⁴C]alanine (0.20 μCi, 400 μM). L-[U-¹⁴C]tyrosine (0.20 μCi, 9.04 μM) is also tested in reconstitution experiments including purified CYP71E1 (Kahn et al, Plant Physiol. 115: 1661-1670, 1997; Bak et al Plant Mol. Biol. 36: 393-405, 1998). Incubations in the shaking water bath for 1 hour at 30° C. are started by addition of substrate (in vivo experiments) or NADPH (in vitro experiments) and stopped by the addition of ethyl acetate. Biosynthetic activity is monitored by the formation of radioactive products using thin layer chromatography (TLC) analysis as previously described (Møller et al, J. Biol. Chem. 254: 8575-8583, 1979) and detection and quantification using a phosphor imager (Storm 840, Molecular Dynamics, Sunnyvale, Calif.). Before TLC application the sample is extracted with ethyl acetate. During this step the surplus of radiolabeled tyrosine remains in the aqueous phase thus preventing overexposure at the origin. The total ethyl acetate phase is applied to the TLC plate. In some experiments, inevitable carry-over of small amounts of the aqueous phase results in the appearance of a tyrosine band at the origin. Unlabeled reference compounds (p-hydroxyphenylacetaldoxime, p-hydroxyphenylacetonitrile and p-hydroxybenzaldehyde) are prestreaked on the TLC plates to permit visual detection under ultraviolet light.

Carbon monoxide binding spectra using intact E. coli cells show the absorption maximum at 450 nm diagnostic for formation of functional cytochrome P450 with the following three constructs: CYP79E1ₙₐ, CYP79E1Δ(1-52)_{2E1(10aa)}, and CYP79E2_{lacZ(24aa)}. The spectra are obtained without and with co-transformation of pSBET but in all cases the cytochrome P450 content turns out to be too low to permit quantification. To obtain an accurate determination, the cytochrome P450s are enriched by isolation of E. coli spheroblasts followed by temperature-induced Triton X-114 phase partitioning (Werck-Reichart et al, Anal. Biochem. 197: 125-131, 1991; Halkier et al, Arch. Biochem. Biophys. 322: 369-377, 1995). The highest expression level (in JM109 cells after 48 hours) of 56 nmol/l culture is obtained using CYP79E2_{lacZ(24aa)}. This level is comparable to the expression level of 62 nmol/l culture obtained with S. bicolor construct CYP79A1Δ(1-33)_{17α(8aa)} (Halkier et al, Arch. Biochem. Biophys. 322: 369-377, 1995) included as a positive control. CYP79E1Δ1-31)_{17α(8aa)} with a modified P45017α N-terminal and the empty vector do not reveal any detectable spectrum.

### Example 10

Reconstitution of CYP79E with CYP71E1

Reconstitution of the membrane associated pathway of cyanogenic glucoside synthesis resulting in the formation of p-hydroxymandelonitrile, the aglycon of dhurrin (seen as p-hydroxybenzaldehyde in vitro) is achieved using enzymes from the two species S. bicolor and Triglochin maritima. In reconstitution experiments including tyrosine, NADPH, NADPH-cytochrome P450 oxidoreductase, CYP71E1 and CYP79E1 or CYP79E2, considerable amounts of p-hydroxyphenylacetonitrile and p-hydroxybenzaidehyde accumulate.

### Example 11

Primers Used in Examples 12 and 13

The following PCR primers are designed on the basis of the genomic Arabidopsis thaliana L. cv. Columbia sequence of CYP79A2 found to be contained in GenBank Accession Number AB010692. Added restriction sites are underlined and sequences encoding CYP17A are indicated in italics:

| A2F1 | 5′-GTGCATATGCTTGACTCCACCCCAATG-3′, | (SEQ ID NO: 3) |
|---|---|---|
| A2R1 . . . | 5′-ATGCATTTTTCTAGTAATCTTTACGCTC-3′, | (SEQ ID NO: 4) |
| A2F2 . . . | 5′-CGTGAATTCCATATGCTCGCGTTTATTATAGGTTTGC-3′, | (SEQ ID NO; 5) |
| A2R2 . . . | 5′-CGGAAGCTTATTAGGTTGGATACACATGT-3′, | (SEQ ID NO: 6) |
| A2R3 . . . | 5′-CGTCACTTGTGCTTTGATCTCTTC-3′, | (SEQ ID NO: 7) |
| A2F3 . . . | 5′-GAACTAATGTTGGCGACGGTTGAT-3′, | (SEQ ID NO: 8) |
| A2FX1 | 5′-CGTGAATTCCATATGGCTCTGTTATTAGCAGTTTTTCTCGCGTTTATTATA- | (SEQ ID NO: 9) |
| | GGTTTG-3′, | |
| A2FX2 | 5′-CGTGAATTCCATATGGCTCTGTTATTAGCAGTTTTTCTTCTTCTTGCATTAAC- | (SEQ ID NO: 10) |
| | TATG-3′, | |
| A2R4 . . . | 5′-CATCTCGAGTCTTCTTCCACTGCTCTCCTT-3′, | (SEQ ID NO: 11) |
| A2FX3 . . . | 5′-TTAATCGGAAACCTACC-3′; | (SEQ ID NO: 12) |
| In addition, the following primers are used | | |
| 17AF | 5′-CGTGAATTCCATATGGCTCTGTTATTAGCTGTT-3′, | (SEQ ID NO: 13) |
| A1R . . . | 5′-GGGCCACGGCACGGGACC-3′, | (SEQ ID NO: 14) |

### Example 12

Cloning of the CYP79A2 cDNA

Using the primers A2F1 and A2R1 PCR is performed on phage DNA representing 2.5×10⁷ pfu of the Arabidopsis thaliana L. (cv. Wassilewskija) silique cDNA library CD4-12 kindly provided by Dr. Linda A. Castle and Dr. David W. Meinke, Department of Botany, Oklohoma State University, Stillwater, Okla., USA, and ABRC. PCR reactions are set up in a total volume of 50 μl in Expand HF buffer with 1.5 mM MgCl₂ (Roche Molecular Biochemicals) supplemented with 200 μM dNTPs, 50 pmol of each primer, and 5% (v/v) DMSO. After incubation of the reactions at 97° C. for 3 min, 2.6 units Expand High Fidelity PCR system (Roche Molecular Biochemicals) are added and 35 cycles of 90 seconds at 95° C., 60 seconds at 65° C. and 120 seconds at 70° C. are run. 0.5 μl of the reaction are subjected to nested PCR using the primers A2F2 and A2R2 and the same PCR conditions. PCR fragments of the expected size are excised from an agarose gel, cloned into EcoRI/HindIII digested pYX223 (R&D Systems), and inserts of 10 clones derived from two nested PCR reactions are sequenced. Sequencing is performed using the Thermo Sequence Fluorescent-labelled Primer cycle sequencing kit (7-deaza dGTP) from Amersham Pharmacia Biotech and analyzed on an ALF-Express DNA Sequencer (Amersham Pharmacia Biotech). Sequence computer analysis is done with programs of the GCG Wisconsin Sequence Analysis Package. The GAP program is used with a gap creation penalty of 8 and a gap extension penalty of 2 to compare pairs of sequences. The splice site prediction is done using NetPlantGene.

CYP79A2 is one of several CYP79 homologues identified in the genome of A. thaliana. According to computer-aided splice site prediction it contains one intron, which is characteristic for A-type cytochromes P450. While it is the only intron in CYP79A2 other members of the CYP79 family have one or two additional introns. The sequence of the full-length CYP79A2 cDNA confirms the splice site prediction. The reading frame of the CYP79A2 cDNA has two potential ATG start codons, one positioned 15 bp downstream of a stop codon in the 5′untranslated region and another one 15 bp further downstream. The cDNA starting with the second ATG codon is for all further studies. This cDNA encodes a protein of 523 amino acids which has 64% similarity and 53% identity to CYP79A1 involved in the biosynthesis of the cyanogenic glucoside dhurrin.

### Example 13

CYP79A2 E. coli Expression Constructs

Expression constructs are derived from a CYP79A2 cDNA obtained by fusion of the two exons amplified from genomic DNA of Arabidopsis thaliana L. The two exons are amplified by PCR with the primers A2F2 and A2R3 for exon 1 and A2F3 and A2R2 for exon2, respectively and using 1.25 units Pwo polymerase (Roche Molecular Biochemicals) and 4 mg template DNA. PCR reactions are set up in a total volume of 50 μl in Pwo polymerase PCR buffer with 2 mM MgSO₄ (Roche Molecular Biochemicals) supplemented with 200 μM dNTPs, 50 pmol of each primer, and 5 (v/v) % DMSO. After incubation of the reactions at 94° C. for 3 minutes, 30 PCR cycles of 20 seconds at 94° C., 10 seconds at 60° C., and 30 seconds at 72° C. are run. After digestion of the PCR fragments with EcoRI (exon 1) and HindIII (exon 2), the blunt ends generated with primers A2R3 and A2F3 and Pwo polymerase are phosphorylated with T4 polynucleotide kinase (New England Biolabs). The two exons are ligated into EcoRI/HindIII digested vector pYX223. The cloned cDNA is sequenced to exclude incorporation of PCR errors.

Four expression constructs are made in the expression vector pSP19g10L (Barnes, Meth. Enzymol. 272: 3-14, 1996):

79A2 (‘native’), wherein 79A2 designates the CYP79A2 coding sequence

17A₍₁₋₈₎79A2 (‘modified’), wherein 17A₍₁₋₈₎ designates a modified N-terminus of CYP17A encoding the amino acid sequence MALLLAVF

17A₍₁₋₈₎79A2Δ(1-8) (‘truncated-modified’), wherein 79A2Δ(1-8) designates the CYP79A2 coding sequence with amino acids 1 to 8 being truncated, and

17A₍₁₋₈₎79A1₍₂₅₋₇₄₎79A2Δ(1-40) (‘chimeric’), wherein 79A1₍₂₅₋₇₄₎ designates amino acids 25 to 74 of CYP79A1 and 79A2Δ(1-40) the CYP79A2 coding sequence with amino acids 1 to 40 being truncated.

N-terminal modifications of CYP79A2 are designed to achieve high-level expression of eukaryotic cytochromes P450 in E. coli. Two constructs are made to introduce the eight N-terminal amino acids of the bovine cytochrome P450 CYP17A in front of the N-terminus of CYP79A2 (yielding ‘modified’ CYP79A2) or a truncated CYP79A2 (yielding ‘truncated-modified’ CYP79A2), respectively. The N-terminus of this cytochrome P450 seems to be especially suitable for expression in E. coli. In a fourth construct (‘chimeric’ CYP79A2) the N-terminal 57 amino acids of CYP79A1Δ(1-24)_{bov} (Halkier et al, Arch Biochem Biophys 322: 369-377, 1995) are fused with the cDNA encoding the catalytic domain (amino acids 41 to 523)of CYP79A2.

The N-terminal modifications are introduced by generating PCR fragments from the ATG start codon to the PstI site of the CYP79A2 cDNA. These fragments are ligated with the PstI/HindIII fragment of the CYP79A2 cDNA and EcoRI/HindIII-digested vector pYX223. For the modified and the truncated modified CYP79A2, the primer pairs A2FX1 and A2R4 as well as A2FX2 and A2R4 are used. The fusion with the N-terminus of CYP79A1 is made by blunt-end ligation of a PCR fragment generated from the CYP79A1Δ(1-25)_{bov} cDNA (Halkier et al, Arch. Biochem. Biophys. 322: 369-377, 1995) using primers 17AF and A1R with a PCR fragment generated from the CYP79A2 cDNA with primers A2FX3 and A2R4. The PCR products are cloned and sequenced to exclude incorporation of PCR errors. The different CYP79A2 cDNAs are excised from pYX223 by digestion with NdeI and HindIII and ligated into NdeI/HindIII-digested pSP19g10L.

### Example 14

CYP79A2 Expression in E. coli

E. coli cells of strain JM109 transformed with the expression constructs described in Example 13 are grown overnight in LB medium supplemented with 100 μg ml⁻¹ ampicillin and used to inoculate 100 ml modified TB medium containing 50 μg ml⁻¹ ampicillin, 1 mM thiamine, 75 μg ml⁻¹ δ-aminolevulinic acid, and 1 mM isopropyl-β-D-thiogalactoside. The cells are grown at 28° C. for 65 hours at 125 rpm. Cells from 75 ml culture are pelleted and resuspended in buffer composed of 0.1 M Tris HCl pH 7.6, 0.5 mM EDTA, 250 mM sucrose, and 250 μM phenylmethylsulfonyl fluoride. Lysozyme is added to a final concentration of 100 μg ml⁻¹. After incubation for 30 minutes at 4° C., magnesium acetate is added to a final concentration of 10 mM. Spheroplasts are pelleted, resuspended in 5 ml buffer composed of 10 mM Tris HCl pH 7.5, 14 mM magnesium acetate, and 60 mM potassium acetate pH 7.4 and homogenized in a Potter-Elvehjem. After DNAse and RNAse treatment, glycerol is added to a final concentration of 29%. Temperature-induced Triton X-114 phase partitioning is performed as described in Halkier et al, Arch Biochem Biophys 322: 369-377, 1995. The Triton X-114 rich phase is analyzed by SDS-PAGE.

Fe²⁺.CO vs. Fe²⁺ difference spectroscopy (Omura et al, J Biol Chem 239: 2370-2378, 1964) is performed on 100 μl E. coli spheroplasts resuspended in 900 μl of buffer containing 50 mM KPᵢ pH 7.5, 2 mM EDTA, 20% (v/v) glycerol, 0.2% (v/v) Triton X-100, and a few grains of sodium dithionite. The suspension is distributed between two cuvettes and a baseline is recorded between 400 and 500 nm on a SLM Aminco DW-2000 ™ spectrophotometer (SLM Instruments, Urbana, Ill.). The sample cuvette is flushed with CO for 1 min and the difference spectrum is recorded. The amount of functional cytochrome P450 is estimated based on an absorption coefficient of 91 l mmol⁻¹ cm⁻¹.

The activity of CYP79A2 is measured in E. coli spheroplasts reconstituted with NADPH:cytochrome P450 oxidoreductase purified from Sorghum bicolor (L.) Moench as described in Sibbesen et al, J Biol Chem 270: 3506-3511, 1995. In a typical enzyme assay, 5 μl spheroplasts and 4 μl NADPH:cytochrome P450 reductase (equivalent to 0.04 units defined as 1 μmol cytochrome c min⁻¹) are incubated with 3.3 μM L-[U-¹⁴C]phenylalanine (453 mCi mmol-⁻¹) in buffer containing 30 mM KPᵢ pH 7.5, 4 mM NADPH, 3 mM reduced glutathione, 0.042% (v/v) Tween 80, and 1 mg ml⁻¹ L-α-dilauroyl phosphatidylcholine in a total volume of 30 μl. To study substrate specificity, 3.7 μM L-[U-¹⁴C]tyrosine (449 mCi mmol⁻¹), 0.1 mM L-[methyl-¹⁴C]methionine (56 mCi mmol⁻¹), and 1 mM L-[5-³H]tryptophan (33 Ci mmol⁻¹), respectively, are used instead of L-[U-¹⁴C]phenylalanine. After incubation at 26° C. for 4 h half of the reaction mixture is analyzed by thin layer chromatography on Silica Gel 60 F₂₅₄ sheets (Merck) using toluene:ethyl acetate (5:1, v/v) as eluent. ¹⁴C radioactive bands are visualized and quantified by STORM 840 PhosphorImager (Molecular Dynamics, Sunnyvale, Calif.). ³H radioactive bands are visualized by autoradiography. Product formation from L-[U-¹⁴C]phenylalanine is linear with time within the first two hours of incubation as determined using time points 30 minutes, 1 hours, 2 hours, and 6 hours. For estimation of Kₘ and Vₘₐₓ values, reaction mixtures are incubated for 2 hours at 26° C. For GC-MS analysis, 450 μl reaction mixture containing 33 μM L-phenylalanine (Sigma) or 33 μM homophenylalanine are incubated for 4 hours at 26° C. and extracted twice with a total volume of ₆₀₀ μl chloroform. The organic phases are combined and evaporated to dryness. The residue is dissolved in 15 μl chloroform and analyzed by GC-MS. GC-MS analysis is performed on an HP5890 Series II gas chromatograph directly coupled to a Jeol JMS-AX505W mass spectrometer. An SGE column (BPX5, 25 m×0.25 mm, 0.25 μm film thickness) is used (head pressure 100 kPa, splitless injection). The oven temperature program is as follows: 80° C. for 3 min, 80° C. to 180° C. at 5° C. min⁻¹, 180° C. to 300° C. at 20° C. min⁻¹, 300° C. for 10 min. The ion source is run in EI mode (70 eV) at 200° C. The retention times of the (E)- and (Z)-isomers of phenylacetaldoxime are 12.43 minutes and 13.06 minutes. The two isomers have identical fragmentation patterns with m/z 135, 117, and 91 as the most prominent peaks.

Protein bands migrating with-an apparent molecular mass of about 60 kDa on SDS-polyacrylamide gels are detected in the detergent-rich phase obtained by temperature-induced Triton X-114 phase partitioning of E. coli spheroplasts harbouring expression constructs for the ‘native’, the ‘truncated-modified’, and the ‘chimeric’ CYP79A2. As expected, the ‘chimeric’ CYP79A2 migrated with a slightly higher molecular mass than the ‘native’ and the ‘truncated-modified’ CYP79A2. No band is detected in the detergent-rich phase from cells harbouring the ‘modified’ CYP79A2 expression construct or the empty vector. Spectral analysis of the different spheroplast preparations shows that the ‘chimeric’ CYP79A2 and to a lesser extend the ‘truncated-modified’ CYP79A2 produce a CO difference spectrum with the characteristic peak at 452 nm indicating the presence of a functional cytochrome P450. A peak at 415 nm is found for all spheroplast preparations. This peak may arise from E. coli derived heme protein, unattached heme groups produced in the presence of δ-aminolevulinic acid in the medium, or cytochrome P450 in a non-functional conformation. Based on the peak at 452 nm, the expression level of ‘chimeric’ CYP79A2 is estimated to be 50 nmol cytochrome P450 (I culture)⁻¹. When incubated with L-[¹⁴C]phenylalanine, spheroplasts of E. coli transformed with the ‘native’, the ‘truncated-modified’, or the ‘chimeric’ CYP79A2 expression construct and reconstituted with the purified NADPH:cytochrome P450 oxidoreductase from S. bicolor produce two radiolabelled compounds which comigrate with the (E)- and (Z)-isomers of phenylacetaldoxime in thin layer chromatography. These products are not detected in assay mixtures containing E. coli spheroplasts harbouring either the ‘modified’ CYP79A2 expression construct or the empty vector. GC-MS analysis shows that two compounds with identical fragmentation patterns are present in the reaction mixture with ‘chimeric’ CYP79A2, but not in the control reaction. The retention times and the fragmentation pattern identify these compounds as the (E)- and (Z)-isomers of phenylacetaldoxime. Administration of L-[¹⁴C]tyrosine, L-[¹⁴C]methionine, or L-[³H]tryptophan to spheroplasts of E. coli expressing the ‘native’ or the ‘chimeric’ CYP79A2 does not result in production of detectable amounts of the respective aldoximes. The ability of CYP79A2 to metabolize DL-homophenylalanine is investigated in spheroplasts of E. coli expressing ‘chimeric’ CYP79A2. GC-MS analysis of the reaction mixture shows the absence of detectable amounts of the homophenylalanine-derived aldoxime. A Kₘ value of 6.7 μmol I⁻¹ and a Vₘₐₓ value of 16.6 pmol min⁻¹ (mg protein)⁻¹ are determined for CYP79A2 using spheroplasts of E. coli expressing ‘native’ CYP79A2 with L-[¹⁴C]phenylalanine as the substrate. As no CO spectrum is obtained with ‘native’ CYP79A2, it is not possible to estimate the amount of functional ‘native’ CYP79A2. However, based on the expression level of functional ‘chimeric’ CYP79A2, a turnover number of 0.24 min⁻¹ for ‘native’ CYP79A2 can be estimated.

The substrate specificity of CYP79A2 seems to be rather narrow as neither L-tyrosine, DL-homophenylalanine, L-tryptophan nor L-methionine are metabolized by the enzyme. The high substrate specificity is in agreement with results obtained with CYP79 homologues involved in the biosynthesis of cyanogenic glucosides, The activity of recombinant CYP79A2 is strongly dependent on the pH of the reaction mixture and, to a lesser extent, on several other factors. Compared to the activity at pH 7.5, the activity of ‘chimeric’ CYP79A2 is 25% at pH 6, 50% at pH 6.5, 80% at pH 7.0, and 70% at pH 7.9. Addition of Tween 80 to a final concentration of 0.083% (v/v) results in a 1.5 fold increase in aldoxime production. Addition of reduced glutathione to a final concentration of 3 mM stimulates aldoxime production, but to a lesser extent.

### Example 15

Constitutive Expression of CYP79A2 in Transgenic Arabidopsis thaliana

Arabidopsis thaliana L. cv. Columbia is used for all experiments. Plants are grown in a controlled-environment Arabidopsis Chamber (Percival AR-60 I, Boone, Iowa, USA) at a photosynthetic flux of 100-120 μmol photons m⁻² sec⁻¹, 20° C. and 70% relative humidity. The photoperiod is 12 hours for plants used for transformation and 8 hours for plants used for biochemical analysis.

For expression of CYP79A2 under control of the CaMV35S promoter in A. thaliana, the native full-length CYP79A2 cDNA is introduced into EcoRI/KpnI digested pRT101 (Töpfer et al, Nucleic Acid Res 15: 5890, 1987) via several subcloning steps. The expression cassette is excised by HindIII digestion and transferred to pPZP111 (Hajdukiewicz et al, Plant Mol Biol 25: 989-994, 1994). Agrobacterium tumefaciens strain C58 (Zambryski et al EMBO J 2: 2143-2150, 1983) transformed with this construct is used for plant transformation by floral dip (Clough et al, Plant J 16: 735-743, 1998) using 0.005% (v/v) Silwet L-77 and 5% (w/v) sucrose in 10 mM MgCl₂. Seeds are germinated on MS medium supplemented with 50 μg ml⁻¹ kanamycin, 2% (w/v) sucrose, and 0.9% (w/v) agar. Transformants are selected after two weeks and transferred to soil.

Rosette leaves (five to eight leaves of different age from each plant) are harvested from six weeks old plants (nine transgenic plants and three wild-type plants), immediately frozen in liquid nitrogen and freeze-dried for 48 hours. Desulfoglucosinolates are analyzed as described by Sørensen (1990) in: Canola and Rapeseed—Production, chemistry, nutrition and processing technology, Shahidi (ed.), Van Nostrand Reinhold, New York, pp 149-172. Briefly, 2 to 5 mg freeze-dried material is homogenized in 3.5 ml boiling 70% (v/v) methanol by a Polytron homogenizer for 1 minute, 10 μl internal standard (5 mM p-hydroxybenzylglucosinolate; Bioraf Denmark) are added, and homogenization is continued for another minute. Plant material is pelleted, and the pellet re-extracted with 3.5 ml boiling 70% (v/v) methanol for 1 minute using a Polytron homogenizer. Plant material is pelleted, washed in 3.5 ml 70% (v/v) methanol and centrifuged. The supernatants are pooled and loaded on a DEAE Sephadex A-25 column equilibrated as follows: 25 mg DEAE Sephadex A-25 are swollen overnight in 1 ml 0.5 M acetate buffer pH 5, packed into a 5 ml pipette tip, and washed with 1 ml water. The plant extract is loaded, and the column is washed with 2 ml 70% (v/v) methanol, 2 ml water, and 0.5 ml 0.02 M acetate buffer pH 5. Helix pomatia sulfatase (Type H-1, Sigma; 0.1 ml, 2.5 mg ml⁻¹ in 0.02 M acetate buffer pH 5) is applied, and the column is left at room temperature for 16 hours. Elution is carried out with 2 ml water. The eluate is dried in vacuo, the residue dissolved in 150 μl water, and 100 μl are subjected to HPLC on a Shimadzu LC-10A Tvp equipped with a Supelcosil LC-ABZ 59142 C₁₈ column (25 cm×4.6 mm, 5 mm; Supelco) and a SPD-M10AVP photodiode array detector (Shimadzu). The flow rate is 1 ml min⁻¹. Elution with water for 2 minutes is followed by elution with a linear gradient from 0 to 60% methanol in water (48 minutes), a linear gradient from 60 to 100% methanol in water (3 minutes) and with 100% methanol (3 minutes). The assignment of peaks is based on retention times and UV spectra compared to standard compounds. Glucosinolates are quantified in relation to the internal standard and by use of the response factors as described by Buchner (1987) In: Glucosinolates in rapeseed: Analytical aspects, Wathelet, (ed.), Martinus Nijhoff Publishers, pp 50-58 and Haughn et al, Plant Physiol 97: 217-226,1991. In the analysis of rosette leaves, the term ‘total glucosinolate content’ refers to the molar amount of the five major glucosinolates (4-methylsulfinylbutylglucosinolate, 4-methylthiobutylglucosinolate, 8-methylsulfinyloctylglucosinolate, indol-3-ylmethylglucosinolate, and 4-methoxyindol-3-ylglucosinolate) which account for 85% of the glucosinolate content in rosette leaves of wild-type A. thaliana and benzylglucosinolate. The glucosinolate content of transgenic seeds harvested from T1 plants #10, #13, and #14 is analyzed and compared with the glucosinolate content of wild-type seeds. Twelve to thirty milligrams of seeds are extracted and subjected to HPLC analysis as described above with the exception that lyophilization of the tissue is omitted. In this analysis of seeds, the term ‘total glucosinolate content’ refers to the molar amount of the ten major glucosinolates (3-hydroxypropylglucosinolate, 4-hydroxybutylglucosinolate, 4-methylsulfinylbutylglucosinolate, 4-methylthiobutylglucosinolate, 8-methylsulfinyloctylglucosinolate, 7-methylthioheptylglucosinolate, 8-methylthiooctylglucosinolate, indol-3-ylmethylglucosinolate, 3-benzoyloxypropylglucosinolate, 4-benzoyloxybutylglucosinolate) which account for more than 90% of the glucosinolate content in seeds of wild-type A. thaliana and benzylglucosinolate.

The appearance of the transgenic plants is comparable to wild-type plants. All transgenic plants (T1 generation) analyzed in the present study accumulate benzylglucosinolate in the rosette leaves while benzylglucosinolate is not detected in simultaneously grown wild-type plants. Benzylglucosinolate is only sporadically observed in roots and cauline leaves of wild-type A. thaliana cv. Columbia and may be induced by environmental conditions. The sporadic occurrence of benzylglucosinolate corresponds with the observation that the CYP79A2 mRNA is a low abundant transcript. CYP79A2 mRNA cannot be detected in seedlings, rosette leaves of different developmental stages, and cauline leaves of A. thaliana cv. Columbia by Northern blotting and RT-PCR. The content of benzylglucosinolate in transgenicplants varies between different plants. In the three plants with highest accumulation, benzylglucosinolate accounted for 38% (plant #10), 5% (plant #14), and 2% (plant #13), respectively, of the total glucosinolate content of the leaves. While seeds of A. thaliana cv. Columbia are known to contain the homophenylalanine-derived 2-phenylethylglucosinolate, the occurrence of benzyiglucosinolate has never been reported for A. thaliana. However, we have detected minute amounts of benzylglucosinolate in seeds of A. thaliana cv. Columbia and cv. Wassilewskija. HPLC analysis of seeds of transgenic plants shows that benzylglucosinolate accounted for 35% (plant #10), 12% (plant #14), and 3% (plant #13) of the total glucosinolate content of the seeds. In seeds of wild-type type plants (cv. Columbia and Wassilewskija) minute amounts of benzylglucosinolate are detected (in cv. Columbia 0.034 μmol (g fresh weight)⁻¹ corresponding to 0.05% of the total glucosinolate content). As indicated by the accumulation of high levels of benzylglucosinolate in several transgenic plants, the formation of phenylacetaldoxime is the rate-limiting step in the biosynthesis of benzylglucosinolate in A. thaliana. The content of the homophenylalanine-derived 2-phenylethylglucosinolate is unaffected in leaves and seeds of the transgenic plants compared to wild-type plants. This supports the data obtained with CYP79A2 expressed in E. coli and shows that CYP79A2 converts specifically phenylalanine, but not homophenylalanine to the corresponding aldoxime.

The nature of the enzymes involved in the conversion of amino acids to aldoximes in the biosynthesis of glucosinolates has been studied in different plant species. It has been proposed that the involvement of cytochrome P450-dependent monooxygenase may be restricted to species which do not belong to the Brassicaceae family implicating that the cytochrome P450-dependent formation of p-hydroxyphenylacetaldoxime in S. alba has to be regarded as a unique exception from the rule or an experimental artifact. The data presented, however, indicate that aldoxime formation from aromatic amino acids is dependent on cytochrome P450 enzymes in members of the Brassicaceae as well as in other families.

### Example 16

Expression Analysis of CYP79A2 by Histochemical GUS Assay

The CYP79A2 promoter is studied in transgenic A. thaliana transformed with a construct containing the CYP79A2 promoter in front of the GUS-intron DNA sequence. A genomic clone containing the CYP79A2 gene is isolated from the EMBL3 genomic library (A. thaliana cv. Columbia). A SacI/XmaI fragment (SEQ ID NO: 15) consisting of 2.5 kB upstream sequence and 120 bp CYP79A2 coding region is excised from the DNA of the positive phage. The fragment is inserted into pPZP111 in frame with the XbaI/SalI fragment of pVictor IV S GiN (Danisco Biotechnology, Denmark) containing the GUS-intron sequence and the 35S terminator. The fusion between the two fragments is made by a 17 bp linker. The resulting transcript encodes a fusion protein consisting of the CYP79A2 membrane anchor fused to the GUS protein.

Transformants of different developmental stages are analyzed by histochemical GUS assays. Intense staining is observed in the veins of the hypocotyl and the petioles of ten days old plants. No staining is seen in the cotelydones and leaves except of the hydathodes where intense staining is observed. In three weeks old plants the veins of the leaves are stained with moderate intensity while intense coloration is observed in the hydathodes. No staining is found in roots of ten days and three weeks old plants. In five weeks old plants no GUS activity is detected.

### Example 17

Arabidopsis Plants and Primers Used in Examples 18, 19, 21, and 22

Arabidopsis cv. Columbia is used for all experiments. Plants are grown in a controlled-environment Arabidopsis Chamber (Percival AR-60 I, Boone, Iowa, USA) at a photosynthetic flux of 100-120 μmol photons m⁻² sec⁻¹, at 20° C. and 70% relative humidity. The photoperiod is 12 hours for plants used for transformation and 8 hours for plants used for biochemical analysis.

Sequences of the PCR primers referred to in the following examples are as follows:

| | | |
|---|---|---|
| T7 | 5′-AAT ACG ACT CAC TAT AG-3′, | (SEQ ID NO: 57) |
| EST3 | 5′-GCT AGG ATC CAT GTT GTA TAC CCA AG-3′, | (SEQ ID NO: 58) |
| EST6 | 5′-CGG GCC CGT TTT CCG GTG GC-3′, | (SEQ ID NO: 59) |
| EST7A | 5′-GGT CAC CAA AGG GAG TGA TCA CGC-3′, | (SEQ ID NO: 60) |
| 5′‘native’ sense | 5′-ATC GTC AGT CGA CCA TAT GAA CAC TTT TAC CTC AAA | (SEQ ID NO: 61) |
| | CTC TTC GG-3′, | |
| 5′‘bovine’ sense | 5′-ATC GTC AGT CGA CCA TAT GGC TCT GTT ATT AGC AGT | (SEQ ID NO: 62) |
| | TTT TAC ATC GTC CTT TAG CAC CTT GTA TCT CC-3′, | |
| 3′‘end’ antisense | 5′-ACT GCT AGA ATT CGA CGT CAT TAC TTC ACC GTC GGG | (SEQ ID NO: 62) |
| | TAG AGA TGC-3′, | |
| CYP79B2.2 | 5′-GGA ATT CAT GAA CAC TTT TAC CTC A-3′, | (SEQ ID NO: 64) |
| B2SB | 5′-TTG TCT AGA TCA CTT CAC CGT CGG GTA-3′, | (SEQ ID NO: 65) |
| B2AF | 5′-GGC CTC GAG ATG AAC ACT TTT ACC TCA-3′, | (SEQ ID NO: 66) |
| B2AB | 5′-TTG GAA TTC CTT CAC CGT CGG GTA GAG-3′, | (SEQ ID NO: 67) |
| XbaI | 5′-GTA CCA TCT AGATTC ATG TTT GTG TAT AGA G-3′, | (SEQ ID NO: 68) |
| EST1 | 5′-TCC ATG TGC TCT ACA TCT-3′, | (SEQ ID NO: 72) |
| EST2 | 5′-GAC GGA ACT CGT ATG TCC-3′, | (SEQ ID NO: 73) |

### Example 18

Cloning of the CYP79B2 and CYP79B5 cDNA and Expression Pattern

EST T42902 identified based on homology to the S. bicolor CYP79A1 lacks 516 base pairs in the 5′ end when compared to CYP79A1. Using the Arabidopsis λPRL2 cDNA library (Newman et al, Plant Physiol. 106: 1241-1255, 1994) as template with the T7 and the gene specific EST3 primer a 255 bp fragment of the missing 5′ end is amplified and subsequently cloned by use of an EcoR I site in the amplified vector sequence and a BamH I site introduced by primer EST3. This fragment is used as template to amplify a Digoxigenin-11 -dUTP (DIG, Boehringer Mannheim) labelled probe (DIG1) by PCR with primers EST6 and EST7A. The λPRL2 library is screened with the DIG1 probe according to the manufacturer's instructions (Boehringer Mannheim) hybridization occurring overnight at 68° C. in 5×SSC, 0.1% N-lauroyl sarcosin, 0.02% SDS, 1.2% (w/v) blocking reagent (Boehringer Mannheim) and stringency washes being performed two times for 15 minutes at 65° C., 0.1×SSC, 0.1% SDS. Detection of positive plaques is done by chemiluminescent detection with nitro blue tetrazolium according to the manufacturer's instructions (Boehringer Mannheim). Screening of the λPRL2 library with the 255 bp PCR fragment as a probe (DIG1) results in the isolation of a full length cDNA clone encoding CYP79B2. EST T42902 is identified based on homology to the S. bicolor CYP79A1 sequence. A 240 bp PCR fragment is amplified with primers EST1 and EST2 using EST T42902 from the Arabidopsis Biological Research Center at OHIO State University as template. This PCR fragment is labelled with Digoxigenin-11-dUTP (DIG, Boehringer Mannheim) and used as probe to screen a lambda ZAP II cDNA library from Brassica napus leaves (Clontech Lab., Inc.). The library is screened with the DIG probe according to the manufacturers instructions, hybridizations occurring overnight at 68° C. in 5×SSC, 0.1% N-lauryl sarcosin, 0.02% SDS, 1.2% (w/v) blocking reagent (Boehringer Mannheim) and stringency washes being performed two times for 15 minutes at 65° C., 0.1×SSC, 0.1% SDS. Positive plaques are detected by chemiluminescent detection with nitro tetrazolium according to the manufacturers instruction (Boehringer Mannheim). Screening of the library results in the isolation of a full length cDNA clone encoding CYP79B5. The sequence reactions are performed using the Thermo Sequence Fluorescent-labelled Primer cycle sequencing kit (Amersham) and analyzed on an ALF-express automated sequenator (Pharmacia). Sequence computer analysis and alignments are produced with programs in the Wisconsin Sequence Analysis Package. For Southern Blot Analysis genomic DNA is isolated from Arabidopsis leaves with the Nucleon PhytoPure Plant DNA extraction kit (Amersham). 10 μg of DNA are digested with BamH I, Xba I, Ssp I, EcoR I or EcoR V and fractionated by gel electrophoresis on a 0.8% agarose gel. Southern blot analysis is performed with the Digoxigenin labelled probe DIG1 and washed under high stringency conditions (68° C., 0.1×SSC, 0.1% SDS, 2×15 minutes). Bands are visualized by chemiluminescent detection with CDP-Star™ (Tropix Inc.). For Northern Blot Analysis total RNA is isolated from rosette leaves, stem leaves, stems, flowers and roots as well as from rosette leaves subjected to wounding. The RNA is isolated using the TRIzol procedure (GibcoBRL). 15 μg of total RNA are separated on a 1% denaturing formaldehyde/agarose gel and blotted onto a positively charged nylon membrane (Boehringer). ³²P-labelled probes covering the entire coding region of CYP79B2 or Arabidopsis ACTIN-1 are produced by random primed labelling. The membrane filter is hybridized in 0.5% SDS, 2×SSC, 5× Denhardt's solution, 20 μg/ml sonicated salmon sperm DNA at 60° C. and excess probe is washed off at 60° C. with 0.2×SSC, 0.1% SDS. Radiolabelled bands are visualized on a Storm 840 phosphorimager and quantified with ImageQuant analysis software.

A start codon is predicted based on the locations of start codons in other CYP79 genes and the most likely sequence surrounding the start codon of dicotelydoneous plants. No stop codon is found 5′ to this start codon. The full length cDNA clones of CYP79B2 and CYP79B5 encode a 61 kDa polypeptide of 541 respectively 540 amino acids length with high homology to other A-type CYP79 cytochromes (Nelson, Arch. Biochem. Biophys 369: 1-10, 1999). Of particular interest are the 93% respectively 96% amino acid identity to Sinapis alba CYP79B1 and the 85% (85%) amino acid identity to Arabidopsis CYP79B3. CYP79B5 is 94% identical to CYP79B2. Generally, CYP79B2 and CYP79B5 show between 44-67% amino acid identity to other known members of the CYP79 family. High stringency Southern Blotting using the DIG1 probe shows that CYP79B2 is a single copy gene. One or two major bands are detected in each lane. This is the general occurrence for A-type cytochrome P450s and correlates with the fact that only a single matching sequence, situated on chromosome IV, has been identified by the Arabidopsis Genome Sequencing Project. However, CYP79B3, which is situated on chromosome II and clustered with several other cytochrome P450s, is 85% identical to CYP79B2 at the amino acid level. It is therefore very likely that CYP79B3 catalyzes the identical reaction. Additional faint bands are detected in most lanes of a southern blot. They are presumably due to hybridization to homologues such as CYP79B3 or the pseudogene CYP79B4. Under low stringency conditions multiple bands are present in each lane, which indicates that multiple CYP79 sequences are present in Arabidopsis. Seven CYP79 homologues have indeed been identified in the Arabidopsis genome sequencing project so far. The expression pattern of CYP79B2 as determined by Northern Analysis of RNA extracted from various Arabidopsis tissues reveils expression in all tissue types examined. The highest level of expression is found in roots, the lowest level in stem leaves; approximately equal amounts are found in rosette leaves, stems and flowers. The level of CYP79B2 messenger RNA in roots is approximately 3-4 fold higher than the level found in rosette leaves. A two-fold induction detectable within 15 minutes after wounding is seen in rosette leaves after 2 hours. Said increase is in agreement with CYP79B2 being involved in indoleglucosinolate biosynthesis.

### Example 19

CYP79B2 E. coli Expression Constructs and Activity Measurement

PCR with the 5′ ‘native’ sense primer or the 5′ ‘bovine’ sense primer against the 3′ ‘end’ antisense primer are used to generate the constructs ‘native’ and ‘Δ(1-9)_{bov}’, respectively, for expression. Using the Aat II and Nde I restriction sites introduced by the primers, the PCR fragments are cloned into an Aat II INde I digested pSP19g10L vector (Barnes, Meth. Enzymol. 272: 3-14, 1996) and sequenced to exclude PCR errors. The native construct consists of the unmodified coding region of CYP79B2, whereas the Δ(1-9)_{bov} construct is truncated by 9 amino acids, in addition to having the first eight codons replaced by the first eight codons of bovine P45017α (17). The bovine modification has been shown to result in high level expression of cytochrome P450s in E. coli. Both constructs carry the modified stop sequence of TAA T to increase translational stop efficiency (Tate et al, Biochem. 31, 2443-2450,1992).

The activity of CYP79B2 is measured by reconstituting spheroplasts from E. coli expressing CYP79B2 with purified NADPH:cytochrome P450 reductase from Sorghum bicolor (L.) Moench. The S. bicolor NADPH:cytochrome P450 reductase is purified as described by Sibbesen et al, J. Biol. Chem. 270: 3506-3511, 1995. The reaction is started by addition of 5 μl of E. coli spheroplasts to a 45 μl reaction mixture containing 100 mM Tricine pH 7.9, 10 μg/μl DLPC (dilaurylphosphatidylcholine) sonicated for 2×10 seconds, 4 mM NADPH, 3 mM reduced glutathiona (GSH), 5 μl [3-¹⁴C]tryptophan (0.1 μCi, specific activity 56.5 mCi/mmol) and 1 U/μl purified NADPH:cytochrome P450 reductase. The reaction is incubated at 34° C. for 30 minutes, extracted two times with ethyl acetate and the ethyl acetate phase is analyzed by TLC using toluen:ethyl acetate 5:1 as eluent. Radiolabelled bands are visualized on a Storm 840 phosphorimager (Molecular Dynamics) and quantified with ImageQuant analysis software (Molecular Dynamics). Substrate specificity is investigated by substituting the ¹⁴C-labelled tryptophan with ¹⁴C-labelled tyrosine or phenylalanine. GC-MS is employed to verify the structure of the compound produced from tryptophan by recombinant CYP79B2. A 450 μl reaction mixture as described above containing 2 mM unlabelled tryptophan is incubated at 34° C. for 2 hours. The reaction mixture is extracted twice with 300 μl CHCl₃ and lyophilized until dryness. GC-MS is performed with an HP5890 Series II gas chromatograph coupled to a Jeol JMS-AX505W mass spectrometer. Splitless injection on an SGE column (BPX5, 25 mm×0.25 mm, 0.25 μm film thickness) and a head pressure of 100 kPa are used. Authentic indole-3-acetaldoxime (IAOX) is synthesized as described by Rausch et al, J. Chromatogr. 318: 95-102, 1985.

### Example 20

CYP79B2 Expression in E. coli

The expression constructs described in Example 19 above are transformed into E. coli strain C43(DE3) (Miroux et al, J. Mol. Biol. 260: 289-298, 1996). Single colonies are grown overnight at 37° C. in LB medium containing 100 μg/ml ampicillin. 1 ml of the overnight culture is used to inoculate 75 ml TB medium containing 100 μg/ml ampicillin, 75 μg/ml δ-aminolevulinic acid, 1 mM thiamine and 1 mM IPTG. The TB cultures are grown for 44 hours at 125 rpm and 28° C. E. coli spheroplasts are prepared as described by Halkier et al, Arch Biochem Biophys 322: 369-377, 1995.

Activity measurements are carried out by reconstituting spheroplasts from E. coli with purified NADPH:cytochrome P450 reductase from S. bicolor in DLPC micelles. Administration of [¹⁴C]tryptophan to reaction mixtures containing spheroplasts from E. coli expressing the native or the Δ(1-9)_{bov} CYP79B2 construct results in the production of a strong band that co-migrates with authentic IAOX standard on TLC. Unambiguous chemical identification of this compound as IAOX is accomplished by GC-MS. No IAOX accumulates in the reaction mixture containing spheroplasts of E. coli transformed with the empty vector. The native construct gives the highest level of activity and thus analyses are performed on recombinant CYP79B2 expressed from this construct. The activity is shown to be dependent on the addition of NADPH:cytochrome P450 reductase since no activity is detected when radiolabelled tryptophan is administered to whole cells. This shows that the endogenous E. coli electron donating system of flavodoxin:NADPH-flavodoxin reductase is not able to donate electrons to CYP79B2. The little activity observed in the absence of NADPH is most likely due to residual amounts of NADPH in the spheroplast preparations. The activity increases 1.8 fold by the addition of 1.5 mM reduced glutathione (GSH). The Kₘ is determined to be 21 μM and Vₘₐₓ is determined to be 97.2 pmol/h/μl spheroplast. No oxime producing activity is detected when radiolabelled phenylalanine or tyrosine are administered to reaction mixtures containing recombinant CYP79B2. This indicates that CYP79B2 is specific for tryptophan. CO-difference spectra of spheroplasts or of the rich phase of a Triton X-114 temperature-induced phase partitioning from the spheroplasts does not show a characteristic peak at 450 nm. Furthermore, when spheroplasts or the Triton X-114 rich phase thereof are separated on an SDS-polyacrylamide gel and stained with Coomassie Brilliant Blue a new band of approximately 60 kD is visible. This indicates that very little recombinant CYP79B2 is produced and that CYP79B2 is highly active. Plasma membrane enzyme systems in Chinese cabbage and Arabidopsis have previously been shown to catalyze the formation of IAOX from tryptophan via a peroxidase-like enzyme (TrpOxE). The conversion is stimulated by H₂O₂ and in certain cases by MnCl₂ and 2,4-dichlorophenol. Addition of 100 mM H₂O₂, 1 mM MnCl₂ or 800 μM 2,4-dichlorophenol to the CYP79B2 reconstitution assays inhibits the activity by 96%, 34% and 72%, respectively, and by 99% when combined. This shows that the two systems are not identical and that the TrpOxE activity is clearly distinctg from CYP79B2. Moreover, a non-enzymatic reaction mixture containing 100 mM H₂O₂, 1 mM MnCl₂ and 800 μM 2,4-dichlorophenol in 50 mM Tricine buffer, pH 8.0 is able to catalyze the conversion of tryptophan to a compound co-migrating with IAOX at a conversion rate of approximately 0.7% of that seen for CYP79B2. This indicates that non-enzymatic conversion of tryptophan to IAOX can occur under oxidative conditions.

### Example 21

Sense and Antisense Expression of CYP79B2 in Arabidopsis thaliana

CYP79B2 cDNA is cloned in sense and antisense direction behind the cauliflower mosaic virus 35S (CaMV35S) promoter using the primers CYP79B2.2, B2SB, B2AF, and B2AB. The native full-length CYP79B2 cDNA is amplified by PCR using the primer pair CYP79B2.2/B2SB (sense construct) and B2AF/B2AB (antisense construct). The PCR product for the sense construct is cloned into EcoR I/Xba I digested pRT101 (Töpfer et al, Nucleic Acid Res 15: 5890, 1987) and sequenced. The PCR product for the antisense construct is cloned into EcoR I/Xho I digested pBluescript (Stratagene), excised by digestion with EcoR I and Kpn I, and ligated into EcoR I/Kpn I digested pRT101 and sequenced. The sense and antisense expression cassettes are excised from pRT101 by Pst I digestion and transferred to pPZP111 (Hajdukiewicz et al, Plant Mol Biol 25: 989-994, 1994). Agrobacterium tumefaciens strain C58 (Zambryski et al, EMBO J 2: 2143-2150, 1983) transformed with either of the constructs is used for transformation of Arabidopsis ecotype Colombia by the floral dip method (Clough et al, Plant J. 16: 735-743, 1998) using 0.005% Silwet L-77 and 5% sucrose in 10 mM MgCl₂. Seeds are germinated on MS medium supplemented with 50 μg/ml kanamycin, 2% sucrose, and 0.9% agar. Transformants are selected after two weeks and transferred to soil.

The glucosinolate profile of transgenic Arabidopsis with altered expression levels of CYP79B2 is analyzed by HPLC as described by Sørensen in: Canola and Rapeseed. Production, Chemistry, Nutrition and Processing Technology, Shahidi, F. (ed.), pp.149-172, 1990, Van Nostrand Reinhold, New York). Glucosinolates are extracted from freeze dried rosette leaves of 6-8 weeks old Arabidopsis by boiling 2×2 minutes in 4 ml 50% ethanol. The extracts are applied to a 200 μl DEAE Sephadex CL-6B column (Pharmacia) equilibrated with 1 ml 0.5 M KOAc, pH 5.0 and washed with 2×1 ml H₂O. The run through is washed out with 3×1 ml H₂O. 400 μl of 2.5 mg/ml sulphatase from Helix pomatia (Sigma-Aldrich) is applied to the column, which is sealed and left overnight. The resulting desulphoglucosinolates are eluted with 2×1 ml H₂O, evaporated until dryness and resuspended in 200 μl H₂O. Aliquots are applied to a Shimadzu Spectachrom HPLC system equipped with a Supelco supelcosil LC-ABZ 59142 C₁₈-column (25 cm×4.6 mm, 5 mm; Supelco) and an SPD-M10AVP photodiode array detector (Shimadzu). The flow rate is 1 ml min⁻¹. Elution with water for 2 minutes is followed by elution with a linear gradient from 0 to 60% methanol in water (48 minutes), a linear gradient from 60 to 100% methanol in water (3 minutes) and with 100% methanol (3 minutes). Detection is performed at 229 nm and 260 nm using a photodiodearray. Desulphoglucosinolates are quantified based on response factors and an internal glucotropaeolin standard.

Arabidopsis plants transformed with antisense constructs of CYP79B2 under control of the 35S promoter have wildtype phenotype whereas the majority (approximately 80%) of the plants transformed with sense constructs of CYP79B2 under control of the 35S promoter exhibit dwarfism. More than 75% of the sense plants develop no inflorescence and give no seeds. The remaining sense plants resemble wildtype plants although seed setting in general is low. The dwarf phenotype of the plants overexpressing CYP79B2 could be due to an increased level of indoleglucosinolates. Overexpression in Arabidopsis of CYP79A1, which converts tyrosine to p-hydroxyphenylacetaldoxime, resulted in dwarfed plants with high content of the tyrosine-derived p-hydroxybenzylglucosinolate. The p-hydroxyphenylacetaldoxime produced by CYP79A1 was very efficiently channelled into p-hydroxybenzylglucosinolate. A similar efficient channelling of IAOX into indoleglucosinolates might also occur in the Arabidopsis overexpressing CYP79B2. However, it cannot be excluded that the dwarf phenotype is due to increased levels of IAA produced from IAOX, or from indole-3-acetonitrile generated from degradation of the increased level of indoleglucosinolates.

HPLC analyses of glucosinolate profiles of the T₁ generation of transgenic Arabidopsis shows that plants overexpressing CYP79B2 accumulate higher quantities of indoleglucosinolates than control plants transformed with empty vector. The levels of the two most abundant indoleglucosinolates glucobrassicin and 4-methoxyglucobrassicin are increased by approximately five fold and two-fold, respectively, whereas the level of neoglucobrassicin is not increased significantly. The total glucosinolate content is increased due to the higher levels of indoleglucosinolates, but the levels of aliphatic and aromatic (i.e. non-indole-) glucosinolates are not affected. In the antisense plants the level of indoleglucosinolates is not reduced compared to control plants. A possible explanation is that the antisense constructs used provide an insufficient means of downregulating CYP79B2. Alternatively, CYP79B3, which based on homology is likely to catalyze the same reaction, compensate the downregulation of indoleglucosinolates.

### Example 22

Expression Analysis of CYP79B2 by Histochemical GUS Assay

Using the DIG system (Boehringer) an Arabidopsis ecotype Columbia EMBL3 genomic library is screened with a 505 bp Digoxigenin-11-dUTP labelled probe annealing to the 5′ end of the CYP79B2 gene. Hybridization of the probe is done at 65° C. in 5×SSC, 0.1% N-lauroylsarcosine, 0.02% SDS, and 1% blocking reagent. Filters are washed in 0.1×SSC, 0.1% SDS at 65° C. prior to detection. Phage DNA from the positive phages is purified as described by Grossberger, Nucleic Acid Res. 15: 6737, 1987. A 5 kb EcoR I fragment, containing the whole CYP79B2 coding region and 2361 bp of the promoter region (see nucleotides 60536 to 62896 of GenBank Accession No. AL035708, SEQ ID NO: 16), is subcloned into pBluescript II SK (Stratagene). An Xba I restriction site is introduced by PCR immediately downstream of the CYP79B2 start codon using the T7 vector primer and the Xba I primer (Example 17). The PCR reaction contains 200 μM dNTPs, 400 pmol of each primer, 0.1 μg template DNA and 10 units Pwo polymerase in a total volume of 200 μl in Pwo polymerase PCR buffer with 2 mM MgSO₄ (Boehringer Mannheim). After incubation of the reactions at 94° C. for 5 minutes, 23 PCR cycles of 30 seconds at 94° C., 30 seconds at 45° C., and 1.5 minutes at 72° C. are run. The resulting PCR product is digested with EcoR I and Xba I, cloned into pBluescript II SK and sequenced to exclude PCR errors. Finally, a transformation plasmid, pPZP111.p79B2-GUS, is constructed by ligating the 2361 bp EcoR I-Xba I fragment of the CYP79B2 promoter region into the binary vector pPZP111 together with the Xba I-Sal I fragment from pVictor IV S GiN (Danisco Biotechnology, Denmark) containing the GUS-intron with 35S terminator. pPZP111.p79B2-GUS is introduced into Agrobacterium tumefaciens C58C1/pGV3850 by electroporation (Wen-Jun et al, Nucleic Acid Res 17: 8385, 1983.

Arabidopsis Ecotype Colombia is Transformed with A. tumefaciens

C58C1/pGV3850/pPZP111 .p79B2-GUS by the floral dip method (Clough et al, Plant J. 16: 735-743, 1998) using 0.005% Silwet L-77 and 5% sucrose in 10 mM MgCl₂. Seeds are germinated on MS medium supplemented with 50 μg/ml kanamycin, 2% sucrose, and 0.9% agar. Transformants are selected after two weeks and transferred to soil. Histochemical GUS assays are performed on T₃ plants essentially as described by Martin et al, in: GUS Protocols: Using the GUS Gene as a Reporter of Gene Expression, Gallagher (ed.), pp 23-43, Academic Press, Inc, with the exception that the tissues are not fixed in paraformaldehyde prior to staining. Tissues are stained for 3 hours.

Highest level of GUS expression is detected in young roots and cotyledons. Some expression is detected in young and mature rosette leaves, where it mainly is associated with the major and minor veins in the vascular tissue. Expression in old leaves is very weak. In siliques, GUS is expressed at the stigmatic surface and where the sepals are attached. There is no detectable GUS staining in the seeds. A very strong GUS staining occurs within 1-2 mm of physical wounds.

### Example 23

Primers Used in Examples 24 and 26

The following PCR primers are designed on the basis of the genomic Arabidopsis thaliana sequence of CYP79F1 found to be contained in GenBank Accession Number AC006341.

| | | |
|---|---|---|
| primer 1 . . . | 5′-CTCTAGATTCGAACATATGGCTAGCTTTACAACATCATTACC-3′, | (SEQ ID NO: 3) |
| primer 2 . . . | 5′-CGGGATCCTTAAGGACGGAACTTTGGATA-3′, | (SEQ ID NO: 4) |
| primer 3 . . . | 5′-AACTGCAGCATGATGAGCTTTACCACATC-3′, | (SEQ ID NO: 5) |
| primer 4 . . . | 5′-CGGGATCCTTAATGGTGGTGATGAGGACGGAACTTTGGATAA-3′, | (SEQ ID NO: 6) |
| primer 5 . . . | 5′-AAAGCTCAATGCGTAGAAT-3′, | (SEQ ID NO: 7) |
| primer 6 . . . | 5′-TTTTTAGACACCATCTTGTTTTCTTCTTC-3′, | (SEQ ID NO: 8) |
| primer 7 . . . | 5′-TGTAGCGGCGCATTAAGC-3′, | (SEQ ID NO: 9) |
| primer 8 . . . | 5′-CAAAAGAATAGACCGAGATAGGG-3′, | (SEQ ID NO: 10) |

### Example 24

CYP79F1 E. coli Expression Constructs

CYP79F1 is one of several CYP79 homologues identified in the genome of A. thaliana. The deduced amino acid sequence of CYP79F1 has 88% identity with the deduced amino acid sequence of CYP79F2 and 43-50% identity with other CYP79 homologues from glucosinolate and cyanogenic glucoside containing species. CYP79F1 and CYP79F2 are located on the same chromosome, only separated by 1638 bp. This suggests that the two genes have been formed by gene duplication and might catalyze similar reactions. The expression construct is derived from the EST ATTS5112 (Arabidopsis Biological Resource Center, Ohio, USA) which contains the full length sequence of CYP79F1. The CYP79F1 coding region is amplified from the EST by PCR using primer 1 (sense direction) and primer 2 (antisense direction). Primer 1 introduces an XbaI site upstream of the start codon and an NdeI restriction site at the start codon. To optimize the construct for E. coli expression (Barnes et al, Proc. Natl. Acad. Sci. USA 88: 5597-5601, 1991) primer 1 changes the second codon from ATG to GCT and introduces a silent mutation in codon 5. Primer 2 introduces a BamHI restriction site immediately after the stop codon. The PCR reaction is set up in a total volume of 50 μl in Pwo polymerase PCR buffer with 2 mM MgSO₄ using 2.5 units Pwo polymerase (Roche Molecular Biochemicals), 0.1 μg template DNA, 200 μM dNTPs and 50 pmol of each primer. After incubation of the reaction at 94° C. for 5 min, 20 PCR cycles of 15 sec at 94° C., 30 sec at 58° C., and 2 min at 72° C. are run. The PCR fragment is digested with XbaI and BamHI, and ligated into the XbaI/BamHI digested vector pBluescript II SK (Stratagene). The cDNA is sequenced on an ALF-Express (Pharmacia) using the Thermo Sequence Fluorescent-labelled Primer cycle sequencing kit (7-deaza dGTP) (Pharmacia) to exclude PCR errors and transferred from pBluescript II SK to an NdeI/BamHI digested pSP19g10L expression vector (Barnes et al, Proc. Natl. Acad. Sci. USA 88: 5597-5601, 1991).

### Example 25

CYP79F1 Expression in E. coli

E. coli cells of strain JM109 (Stratagene) and strain C43(DE3) (Miroux et al, J. Mol. Biol. 260: 289-298, 1996) transformed with the expression construct are grown overnight in LB medium supplemented with 100 μg ml⁻¹ ampicillin and used to inoculate 40 ml modified TB medium containing 50 μg ml⁻¹ ampicillin, 1 mM thiamine, 75 μg ml⁻¹ δ-aminolevulinic acid, 1 μg ml⁻¹ chloramphenicol and 1 mM isopropyl-β-D-thiogalactoside. The cultures are grown at 28° C. for 60 hours at 125 rpm. The cells are pelleted and resuspended in buffer composed of 0.2 M Tris HCl, pH 7.5, 1 mM EDTA, 0.5 M sucrose, and 0.5 mM phenylmethylsulfonyl fluoride. Lysozyme is added to a final concentration of 100 μg ml⁻¹. After incubation for 30 minutes at 4° C., Mg(OAc)₂ is added to a final concentration of 10 mM. Spheroplasts are pelleted, resuspended in 3.2 ml buffer composed of 10 mM Tris HCl, pH 7.5, 14 mM Mg(OAc)₂, and 60 mM KOAc, pH 7.4 and homogenized in a Potter-Elvehjem homogenizer. After DNase treatment, glycerol is added to a final concentration of 30%. Temperature-induced Triton X-114 phase partitioning results in the formation of a detergent rich-phase containing the majority of the cytochrome P450 and a detergent poor-phase (Halkier et al, Arch. Biochem. Biophys. 322: 369-377, 1995). Functional expression of CYP79F1 is monitored by Fe²⁺.CO vs. Fe²⁺ difference spectroscopy (Omura et al, J. Biol. Chem. 239: 2370-2378, 1964) performed on an SLM Aminco DW-2000 ™ spectrophotometer (SLM Instruments, Urbana, Ill.) using 10 μl Triton X-114 rich-phase in 990 μl of buffer containing 50 mM KPᵢ, pH 7.5, 2 mM EDTA, 20% glycerol, 0.2% Triton X-100, and a few grains of sodium dithionite.

The activity of CYP79F1 is measured in E. coli spheroplasts reconstituted with NADPH:cytochrome P450 oxidoreductase purified from Sorghum bicolor (L.) Moench as described by Sibbesen et al, J. Biol. Chem. 270: 3506-3511, 1995. In a typical enzyme assay, 5 μl spheroplasts and 4 μl NADPH:cytochrome P450 reductase (equivalent to 0.04 units defined as 1 μmol cytochrome c/min) are incubated with substrate in buffer containing 30 mM KPᵢ, pH 7.5, 3 mM NADPH, 3 mM reduced glutathione, 0.042% Tween 80, 1 mg ml⁻¹ L-α-dilauroylphosphatidylcholine in a total volume of 30 μl. Reaction mixtures containing spheroplasts of E. coli C43(DE3) transformed with empty vector are used as controls in all assays. 3.3 μM L-[U-¹⁴C]phenylalanine (453 mCi/mmol; Pharmacia), 3.7 μM L-[U-¹⁴C]tyrosine (449 mCi/mmol; Pharmacia), 0.1 mM L-[methyl-¹⁴C]methionine (56 mCi/mmol; Pharmacia), and 24 μM L-[side chain-3-¹⁴C]tryptophan (56.5 mCi/mmol; NEN) are tested as potential substrates. After incubation at 28° C. for 1 hour, half of the reaction mixture is analyzed by TLC on Silica Gel 60 F₂₅₄ sheets (Merck) using toluene/ethyl acetate 5:1 (v/v) as eluent. Radiolabelled bands are visualized and quantified using a STORM 840 phosphoimager (Pharmacia). For GC-MS analysis, 450 μl reaction mixture containing 3.3 mM L-methionine (Sigma), 3.3 mM DL-dihomomethionine or 3.3 mM DL-trihomomethionine, respectively, are incubated for 4 hours at 25° C. and extracted with a total volume of 600 μl CHCl₃. The organic phase is collected, evaporated, and the residue is dissolved in 15 μl CHCl₃ and analyzed by GC-MS. GC-MS analysis is performed on an HP5890 Series II gas chromatograph directly coupled to a Jeol JMS-AX505W mass spectrometer. An SGE column (BPX5, 25 m×0.25 mm, 0.25 μm film thickness) is used (heat pressure 100 kPa, splitless injection). The oven temperature program is as follows: 80° C. for 3 minutes, 80° C. to 180° C. at 5° C. min⁻¹, 180° C. to 300° C. at 20° C. min⁻¹, and 300° C. for 10 min. The ion source is run in EI mode (70 eV) at 200° C. The retention times of the E- and Z-isomer of 5-methylthiopentanaldoxime are 14.3 min and 14.8 min, respectively. The two isomers have identical fragmentation patterns with m/z values of 130, 129, 113, 82, 61 and 55 as the most prominent peaks. The retention times of the E- and Z-isomer of 6-methylthiopentanaldoxime are 17.1 min and 17.6 min, respectively. The two isomers have identical fragmentation patterns with m/z values of 144, 143, 98, 96, 69, 61 and 55 as the most prominent peaks. DL-dihomomethionine, DL-trihomomethionine, 5-methylthiopentanaldoxime and 6-methylthiohexanaldoxime are synthesized as described (Dawson et al, J. Biol. Chem. 268: 27154-27159, 1993) and authenticated by NMR spectroscopy.

A CO difference spectrum with the characteristic peak at 450 nm is obtained for CYP79F1 expressed in E. coli strain C43(DE3), but not for CYP79F1 expressed in E. coli strain JM109. In addition to the peak at 450 nm, a peak at 418 nm is detected. To identify substrates of CYP79F1, activity measurements are carried out using spheroplasts of E. coli C43(DE3) reconstituted with NADPH:cytochrome P450 reductase from S. bicolor. When the reaction mixture containing CYP79F1 is incubated with DL-dihomomethionine, two compounds, which are not present in the control reactions, are detected by GC-MS. The retention times and the mass spectral fragmentation patterns of these compounds are identical with those for the E/Z-isomers of synthetic 5-methylthiopentanaldoxime. When DL-trihomomethionine is administred to the reaction mixture containing CYP79F1, two compounds with retention times and fragmentation pattern identical with those of the E/Z-isomers of the synthetic 6-methylthiopentanaldoxime are detected by GC-MS. Administration of L-methionine, L-phenylalanine, L-tyrosine, and L-tryptophan to the reaction mixtures containing recombinant CYP79F1, did not result in the formation of detectable amounts of the corresponding aldoximes.

### Example 26

Expression of CYP79F1 cDNA in Transgenic Arabidopsis thaliana

Arabidopsis thaliana L. cv. Columbia is used for all experiments. Plants are grown in a controlled-environment Arabidopsis Chamber (Percival AR-60 I, Boone, Iowa, USA) at a photosynthetic flux of 100-200 μmol photons m-⁻² sec-⁻¹, 20° C. and 70% relative humidity. Unless otherwise stated the photoperiod is 12 hours for plants used for transformation and 8 hours for plants used for biochemical analysis.

Generation of Transgenic Plants

To construct plants which express the CYP79F1 cDNA under control of the CaMV 35S promoter (35S:CYP79F1 plants), the CYP79F1 cDNA is PCR amplified from the EST ATTS5112 (Arabidopsis Biological Resource Center, Ohio, USA) using primer 3 (sense direction) and primer 4 (antisense direction). Primer 3 is tailed with a PstI restriction site. Primer 4 introduces 4 codons coding for His before the stop codon and a BamHI restriction site after the stop codon. The PCR fragment containing the CYP79F1 cDNA is digested with PstI and BamHI, ligated into the PstI/BamHI digested vector pBluescript II SK and sequenced to exclude PCR errors. The CYP79F1 cDNA is placed under control of the CaMV 35S promoter by ligation into the PstI/BamHI digested vector pSP48 (Danisco Biotechnology, Denmark). The expression cassette is excised by XbaI digestion and transferred to pPZP111 (Hajdukiewicz et al, Plant Mol. Biol. 25: 989-994, 1994). Agrobacterium tumefaciens strain C58 (Zambryski et al, EMBO 2: 2143-2150, 1983) transformed with this construct is used for plant transformation by floral dip (Clough et al, Plant J. 16: 735-743, 1998) using 0.005% Silwet L-77 and 5% sucrose in 10 mM MgCl₂. Seeds are germinated on MS medium supplemented with 50 μg ml⁻¹ kanamycin, 2% sucrose, and 0.9% agar. Transformants are selected after two weeks and transferred to soil.

Nine primary 35S:CYP79F1 transformants are investigated. Three plants (S5, S7, S9) differ morphologically from wild-type plants. These plants have reduced growth rates, but a normal appearance within the first seven weeks of growth. Before floral transition becomes apparent, reduced apical dominance results in production of multiple axillary shoots which later developed into lateral inflorescences. These morphological changes give S5, S7 and S9 a bushy phenotype. In addition, S5 has curly rosette leaves with the leaf tips bending downwards. Transgenic A. thaliana plants with altered content of aliphatic glucosinolates due to co-suppression or over-expression of CYP79F1 possess a characteristic morphological phenotype characterized by prolonged vegetative growth and production of multiple axillary shoots. A. thaliana has been reported to be able to tolerate overexpression of cytochromes P450 of the CYP79 family leading to a two to five fold increase in glucosinolate content without similar changes in the appearence of the plants. Therefore it seems unlikely that the morphological changes result from the presence or absense of specific glucosinolates. A possible explanation is that the morphological phenotype is due to a pleiotropic effect caused by disturbance of the plant's sulfur metabolism, in which methionine plays a central role. Alterations of the methionine metabolism may explain why both plants with co-suppression and overexpression of CYP79F1 show similar morphological changes when compared to wild-type plants. The onset of the morphological changes in CYP79F1 co-suppressed plants at the time of floral transition may be due to the requirement for methionine to support flower development. Alternatively, it coincides with an increase in the level of CYP79F1 expression in wild-type plants.

HPLC Analysis of the Glucosinolate Content of Plant Extracts

Six to eight rosette leaves from each plant are harvested from nine 9-week-old primary transformants of 35S:CYP79F1 plants and ten 7-week-old wild-type plants of the same size. The tissue is immediately frozen in liquid nitrogen and freeze-dried for 48 hours. Glucosinolates are analyzed as desulfoglucosinolates as follows: 3.5 ml of boiling 70% (v/v) methanol are added to 9 to 20 mg freeze-dried material, 10 μL internal standard (5 mM p-hydroxybenzylglucosinolate; Bioraf, Denmark) are added, and the sample is incubated in a boiling water bath for 4 min. Plant material is pelleted, the pellet is re-extracted with 3.5 ml 70% (v/v) methanol and centrifuged. The supernatants are pooled and analyzed by HPLC after sulfatase treatment as described by Wittstock et al, J. Biol. Chem. 275, 14659-14666, 2000. The assignment of peaks is based on retention times and UV spectra compared to standard compounds. Glucosinolates are quantified in relation to the internal standard and by use of response factors (Haughn et al, Plant Physiol. 97: 217-226, 1991; Buchner in: Glucosinolates in rapeseed: Analytical aspects., Wathelet (ed), Martinus Nijhoff Publisher, Boston, pp. 155-181, 1987). The term ‘total glucosinolate content’ refers to the molar amount of the seven major glucosinolates (3-methylsulfinylpropylglucosinolate, 4-methylsulfinylbutylglucosinolate, 4-methylthiobutylglucosinolate, 8-methylsulfinyloctylglucosinolate, indol-3-ylmethylglucosinolate, 4-methoxyindol-3-ylglucosinolate, and N-methoxyindol-3-ylglucosinolate) which account for more than 85% of the glucosinolate content in rosette leaves of wild-type A. thaliana.

The dihomomethionine-derived glucosinolates 4-methylsulfinylglucosinolate and 4-methylthiobutylglucosinolate account for more than 50% of the total glucosinolate content of leaves of A. thaliana whereas glucosinolates derived from trihomomethionine are only minor constituents of the leaves (2.1% of the total glucosinolate content. Accordingly the analysis focuses on 4-methylsulfinylbutylglucosinolate and 4-methylthiobutylglucosinolate.

Three plants (S1, S7, S9) show dramatically reduced levels of 4-methylsulfinylbutyl-glucosinolate and 4-methylthiobutylglucosinolate in rosette leaves while two plants (S3, S5) have slightly increased levels of these glucosinolates. The content of 4-methylsulfinylbutyl-glucosinolate and 4-methylthiobutylglucosinolate is reduced to 0.7, 2.2 and 2.8 μmol (g dw)⁻¹ in S7, S1 and S9, respectively, and increased to 12.3 and 13.3 μmol (g dw)⁻¹ in S3 and S5, respectively, as compared to a level ranging from 5.7 to 11.5 μmol (g dw)⁻¹ in wild-type plants. The levels of 4-methylsulfinylbutylglucosinolate and 4-methylthiobutyl-glucosinolate are influenced equally. Since aldoxime formation from dihomomethionine is believed to precede the secondary modification which determines the ratio between the amounts of 4-methylsulfinylbutylglucosinolate and 4-methylthiobutylglucosinolate, the total amount of both glucosinolates reflects the alterations in the activity of upstream enzymes. The reduced levels of 4-methylsulfinylbutylglucosinolate and 4-methylthiobutylglucosinolate indicated that co-suppression of CYP79F1 occurs in S1, S7 and S9. The slight increase of the content of 4-methylsulfinylbutylglucosinolate and 4-methylthiobutylglucosinolate in S3 and S5 indicates an increased expression level of CYP79F1. This suggests that the chain-elongation of methionine is a rate limiting step in the biosynthesis of aliphatic glucosinolates. It can, however, not be excluded that the low level of accumulation may be the result of a low expression level of the transgene due to position effects with respect to integration of the T-DNA. As the dihomomethionine-derived glucosinolates are the major glucosinolates of wild-type rosette leaves, altered levels of these glucosinolates influence the total glucosinolate content remarkably. This is particularly pronounced in the plants with CYP79F1 co-suppression. These plants have a total glucosinolate content ranging from 4.3 to 4.8 μmol (g dw)⁻¹ as compared to the total glucosinolate content of wild-type plants ranging from 8.8 to 17.4 μmol (g dw)⁻¹. In addition to the changes in the content of 4-methylsulfinylbutylglucosinolate and 4-methylthiobutyl-glucosinolate, alterations in the level of other glucosinolates, particularly of Methionine-derived glucosinolates, are observed in 35S:CYP79F1 plants. Plants with a reduced content of 4-methylsulfinylbutylglucosinolate and 4-methylthiobutylglucosinolate also have reduced levels of the other major glucosinolates derived from chain-elongated methionine homologues, i.e. 3-methylsulfinylpropylglucosinolate and 8-methylsulfinyloctylglucosinolate. This might be explained by co-suppression not only of the CYP79F1 transcript but also of transcripts of other CYP79 homologues involved in the biosynthesis of aliphatic glucosinolates such as transcripts of CYP79F2 which has 88% amino acid identity with CYP79F1. Alternatively, it might reflect that CYP79F1 has a broad substrate specificity for chain-elongated methionines. The fact that chain-elongated methionines accumulate in plants with CYP79F1 co-suppression indicates that the enzymes catalyzing the chain elongation of methionine are not subject to feedback inhibition by the chain-elongated product. The content of the three indoleglucosinolates is not affected significantly.

Analysis of the Amino Acid Content of Plant Extracts

Rosette leaves from three 12-week-old primary transformants of 35S:CYP79F1 plants and three 8-week-old wild-type plants of the same size are used. 250 mg of leaf material from each plant are homogenized in 3 ml 50 mM KPᵢ, pH 7.5 using a Polytron homogenizer. The plant material is pelleted (20000 g for 10 minutes) and re-extracted twice with 3 ml 50 mM KPᵢ, pH 7.5. The water phases are combined, dried in vacuo, and the residue is dissolved in 100 μl water. An aliquot of the redissolved extract is treated with 1/10 volume 30% salicylic sulfonic acid and denatured proteins are removed by centrifugation. The supernatant is neutralized with 1/10 volume 1 N NaOH. The individual protein amino acids in the sample are identified and quantified using an Ultropac 8 Resin Reverse Phase HPLC column (200×4.6 mm) on a Biochrom 20 amino acid analyzer (Pharmacia) essentially according to the manufacturer's elution program.

For quantification of dihomomethionine in plant material, the sample is subjected to two elution programs slightly modified from the program recommended by the manufacturer. Program 1 is as follows: 53° C. for 7 minutes, buffer A; 50° C. for 35 minutes, buffer A; 95° C. for 34 minutes, buffer A. Program 2 is as follows: 53° C. for 7 minutes, buffer A; 58° C. for 12 minutes, buffer B; 95° C. for 25 minutes, buffer C. Buffer A is 0.2 M sodium citrate, pH 3.25, buffer B is 0.2 M sodium citrate, pH 4.25, and buffer C is 1.2 M sodium citrate, pH 6.25. In program 1, phenylalanine and dihomomethionine co-elute at 63.6 minutes. In program 2, tyrosine and dihomomethionine co-elute at 25.3 minutes. Dihomomethionine is quantified as the difference between the peak area corresponding to phenylalanine and dihomomethionine in program 1 and the peak area corresponding to phenylalanine in program 2, and as the difference between the peak area corresponding to tyrosine and dihomomethionine in program 2 and the peak area corresponding to tyrosine in program 1. The response factor for dihomomethionine is determined using an authentic standard.

For quantification of trihomomethionine in the plant material, the sample is also subjected to an elution program slightly modified from the program recommended by the manufacturer. Program 3 is as follows: 53° C. for 7 minutes, buffer A; 58° C. for 5 minutes, buffer B; 95° C. for 7 minutes, buffer B; 95° C. for 25 minutes, buffer C. Trihomomethionine elutes at 29.0 minutes and is quantified as the peak area using a response factor determined with an authentic standard.

Analysis of the content of dihomo- and trihomomethionine in S7, the 35S:CYP79F1 plant with the most significant reduction in the glucosinolate content and a strong morphological phenotype, reveals a 50 fold increase compared to wild-type plants. Trihomomethionine accumulates to fourfold of the content in wild-type plants. In S9 a 15 fold increase of the dihomomethionine content is observed whereas no increase of the trihomomethionine content is detected.

Expression Analysis by RT-PCR

To check for inhibition of RT reactions by components of RNA preparations obtained from different plant tissues control RNA is used which is synthesized from the pBluescript II SK vector (Stratagene) linearized by digestion with ScaI. The synthesis reaction is set up in a total volume of 100 μl in Transcription Optimized Buffer (Promega) supplemented with 500 μM rNTPs, 10 mM DTT, 100 units RNAsin Ribonuclease inhibitor (Promega), 3 μg linearized pBluescript II SK, and 50 units T3 RNA polymerase (Promega). After incubation at 37° C. for 2 hours, 20 units of RNase-free DNase are added, and the reaction is incubated at 37° C. for another 1 hour. Following extraction with phenol and CHCl₃ and precipitation with ethanol, the RNA is dissolved in diethylpyrocarbonate-treated water.

The following tissues are harvested from A. thaliana:

(1) total plant tissue of 4-week-old plants (grown at 8 hours light/16 hours dark);

(2) rosette leaves (without petioles) and

(3) above ground parts of 5-week-old plants (before onset of floral transition; grown at 8 hours light/16 hours dark);

(4) rosette leaves (without petioles) and

(5) cauline leaves of flowering plants (9 weeks old; grown at 12 hours light/12 hours dark to induce flowering).

Total RNA is isolated from said tissuey using TRIZOL-Reagent (GIBCO BRL). The RNA is quantified spectrophotometrically and used to synthesize first-strand cDNA. To ensure linearity of the RT-PCR, first-strand cDNA synthesis is performed on 1 μg, 0.3 μg and 0.1 μg of each pool of RNA.The cDNA is synthesized in First Strand Buffer (GIBCO BRL) supplemented with 0.5 mM dNTPs, 10 mM DTT, 200 ng random hexamers (Pharmacia), 3 pg control RNA (internal standard), and 200 units SUPERSCRIPTII Reverse transcriptase (GIBCO BRL) in a total volume of 20 μl. The reaction mixture is incubated at 27° C. for 10 minutes followed by incubation at 42° C. for 50 minutes and inactivation at 95° C. for 5 minutes. The RT-reactions are purified by means of a PCR-purification kit (QIAGEN; elution with 50 μl of 1 mM Tris-buffer, pH 8). 2 μl of the purified RT-reactions are subjected to PCR. The PCR reactions are set up in a total volume of 50 μl in PCR buffer (GIBCO BRL) supplemented with 200 μM dNTPs, 1.5 mM MgCl₂, 50 pmol of sense primer, 50 pmol of antisense primer, and 2.5 units Platinum Taq DNA polymerase (GIBCO BRL). The PCR program is as follows: 2 minutes at 94° C., 32 cycles of 30 seconds at 94° C., 30 seconds at 57° C., 50 seconds at 72° C. 10 μl of the PCR reactions are analyzed by gel electrophoresis on 1% agarose gels. Bands are visualized by ethidium bromide staining and quantified on a Gel Doc 2000 Transilluminator (Biorad). The primers used to analyze the CYP79F1 transcript are primer 5 (sense direction) and primer 6 (antisense direction). At 57° C. primer 5 does not anneal to genomic DNA comprising the CYP79F1 gene as the sequence of primer 5 is complementary to the sequences flanking an 111 bp intron of the CYP79F1 gene. Primer 6 anneals to the 3′-untranslated region of CYP79F1 and is highly specific for CYP79F1. The primers used to analyze the internal standard are primer 7 (sense direction) and primer 8 (antisense primer). PCR analysis of the internal standard shows that the RT reactions run with the same efficiency in samples prepared with different amounts of RNA isolated from different plant tissues.

A CYP79F1 transcript is detected in all tissues examined. The transcript level increases with maturation of the plants. The expression level is approximately four times higher in rosette leaves of 9-week-old flowering plants than in rosette leaves of 5-week-old plants. When the above ground parts of 5-week-old plants are analyzed, less CYP79F1 transcript is detected than in rosette leaves of the same plants. This indicates that CYP79F1 is expressed at higher levels in rosette leaves than in petioles.

What is claimed is:

1. A DNA coding for a P450 monooxygenase converting an aliphatic or aromatic amino acid or chain-elongated methionine homologue to the corresponding oxime.

2. The DNA of claim 1 converting L-Valine or L-Isoleucine to the corresponding oxime; tyrosine to p-hydroxyphenylacetaldoxime; L-phenylalanine to phenylacetaldoxime; tryptophan to indole-3-acetaldoxime; or chain-elongated methionine to the corresponding oxime.

3. The DNA of claim 1 coding for a P450 monooxygenase consisting of amino acid residues independently selected from the group of the amino acid residues Gly, Ala, Val, Leu, lIe, Phe, Pro, Ser, Thr, Cys, Met, Trp, Tyr, Asn, Gln, Asp, Glu, Lys, Arg and His, wherein global alignment of the amino acid sequence of the encoded protein shows at least 40% identity to the amino acid sequence resulting from the global alignment with SEQ ID NO: 1 or SEQ ID NO: 3or both; SEQ ID NO: 39; or SEQ ID NO: 54 or SEQ ID NO: 70 or both; or at least 50% identity to the amino acid sequence resulting from the global alignment with SEQ ID NO: 9 or SEQ ID NO: 11 or both or SEQ ID NO: 74 or SEQ ID NO: 84 or both.

4. The DNA of claim 1, wherein an open reading frame is operably linked to one or more regulatory sequences different from the regulatory sequences associated with the genomic gene containing the exons of the open reading frame.

5. The DNA of claims 1 to 4 coding for a P450 monooxygenase having the formula R₁-R₂-R₃, wherein
R₁, R₂ and R₃ designate component sequences, and
R₂ consists of 150 to 175 or more amino acid residues the sequence of which is at least 60% to 65% identical to an aligned component sequence of SEQ ID NO: 1 or SEQ ID NO: 3; SEQ ID NO: 9 or SEQ ID NO: 11; SEQ ID NO: 39; SEQ ID NO: 54 or SEQ ID NO: 70; or SEQ ID NO: 74 or SEQ ID NO: 84.

6. The DNA of claim 1, wherein the amino acid sequence of R₂ is represented by
amino acids 334-484 of SEQ ID NO: 1 or amino acids 333-483 of SEQ ID NO: 3;
amino acids 339-489 of SEQ ID NO: 9 or amino acids 332-482 of SEQ ID NO: 11;
amino acids 308-487 of SEQ ID NO: 39;
amino acids 196-345 of SEQ ID NO: 54 or amino acids 192-341 of SEQ ID NO: 70;
amino acids 334-483 of SEQ ID NO: 74 or amino acids 332-481 of SEQ ID NO: 84.

7. The DNA of claim 1 coding for a P450 monooxygenase of 450 to 600 amino acid residues length.

8. The DNA of claim 1 coding for a P450 monooxygenase having the amino acid sequence of SEQ ID NO: 1 or SEQ ID NO: 3; SEQ ID NO: 9 or SEQ ID NO: 11; SEQ ID NO: 39; SEQ ID NO: 54 or SEQ ID NO: 70; SEQ ID NO: 74 or SEQ ID NO: 84.

9. The DNA of claim 1 having the nucleotide sequence of SEQ ID NO: 2 or SEQ ID NO: 4; SEQ ID NO: 9 or SEQ ID NO: 12; SEQ ID NO: 40; SEQ ID NO: 75 or SEQ ID NO: 85.

10. A P450 monooxygenase converting an aliphatic or aromatic amino acid or a chain-elongated methionine homologue to the corresponding oxime as coded for by the DNA of any one of claims 1 to 7.

11. A plant wherein the genomic DNA comprises and expresses the DNA of claim 4.

12. A method for the isolation of a cDNA coding for a P450 monooxygenase converting an aliphatic or aromatic amino acid or chain-elongated methionine to the corresponding oxime; comprising
(a) preparing a cDNA library from plant tissue expressing such a monooxygenase,
(b) using at least one oligonucleotide designed on the basis of SEQ ID NO: 1, SEQ ID NO: 2, SEQ ID NO: 3, or SEQ ID NO: 4; SEQ ID NO: 9, SEQ ID NO: 10, SEQ ID NO: 11, or SEQ ID NO: 12;; SEQ ID NO: 39 and SEQ ID NO: 40; SEQ ID NO: 54, SEQ ID NO: 55, SEQ ID NO: 56, SEQ ID NO: 70 or SEQ ID NO: 71; or SEQ ID NO: 74, SEQ ID NO: 75, SEQ ID NO: 84 or SEQ ID NO: 85 to amplify part of the P450 monooxygenase cDNA from the cDNA library,
(c) optionally using a further oligonucleotide designed on the basis of SEQ ID NO: 1, SEQ ID NO: 2, SEQ ID NO: 3, or SEQ ID NO: 4; SEQ ID NO: 9, SEQ ID NO: 10, SEQ ID NO: 11, or SEQ ID NO: 12;; SEQ ID NO: 39 and SEQ ID NO: 40; SEQ ID NO: 54, SEQ ID NO: 55, SEQ ID NO: 56, SEQ ID NO: 70 or SEQ ID NO: 71; or SEQ ID NO: 74, SEQ ID NO: 75, SEQ ID NO: 84 or SEQ ID NO: 85 to amplify part of the P450 monooxygenase cDNA from the cDNA library in a nested PCR reaction,
(d) using the DNA obtained in steps (b) or (c) as a probe to screen a cDNA library prepared from plant tissue expressing a P450 monooxygenase converting an aliphatic or aromatic amino acid or chani-elongated methinone honologue to the corresponding oxime, and
(e) identifying and purifying vector DNA comprising an open reading frame encoding a protein characterized by an amino acid sequence showing at least 40% identity to the amino acid sequence resulting from the global alignment with SEQ ID NO: 1 or SEQ ID NO: 3 or both; SEQ ID NO: 39; SEQ ID NO: 54 or SEQ ID NO: 70 or both; or at least 50% identity to the amino acid sequence resulting from the global alignment with SEQ ID NO: 9 or SEQ ID NO: 11 or both; or SEQ ID NO: 74 or SEQ ID NO: 84 or both;
(f) optionally further processing the purified DNA.

13. A marker assisted breeding method selecting plants with a desired trait using hybridization with one or more oligonucleotides, wherein the sequence of at least one of said oligonucleotides constitutes a component sequence of the DNA of claim 1.

14. A method for producing purified recombinant P450 monooxygenase converting an aliphatic or aromatic amino acid or chain-elongated methionine homologue to the corresponding oxime, comprising expression of a corresponding gene in P. pastoris.

15. A method for obtaining a transgenic plant, comprising
(a) stably integrating into a plant cell or tissue which can be regenerated to a complete plant DNA comprising at least part of an open reading frame of a P450 monooxygenase converting an aliphatic or aromatic amino acid or chain-elongated methionine homologue to the corresponding oxime, and
(b) selecting transgenic plants.

16. The method of claim 15 resulting in transgenic expression of a P450 monooxygenase in a plant.

17. The method of claim 15 resulting in the reduced expression of an endogenous P450 monooxygenase in a plant.

18. The method of claim 15 resulting in an altered content or profile of cyanogenic glucosides or glucosinolates.


SEQUENCE LISTING (85 sequences)

SEQ ID NO: 1 - protein, 542 aa, Manihot esculenta
MAMNVSTTIGLLNATSFASSSSINTVKILFVTLFISIVSTIVKLQKSAANKEGSKKLPLPPGPTPWPLIGNIPEMIRYRPTFRWIHQLMKDMNTDICLIRFGRTNFVPISCPVLAREILKKNDAIFSNRPKTLSAKSMSGGYLTTIVVPYNDQWKKMRKILTSEIISPARHKWLHDKRAEEADNLVFYIHNQFKANKNVNLRTATRHYGGNVIRKMVFSKRYFGKGMPDGGPGPEEIEHIDAVFTALKYLYGFCISDFLPFLLGLDLDGQEKFVLDANKTIRDYQNPLIDERIQQWKSGERKEMEDLLDVFITLKDSDGNPLLTPDEIKNQIAEIMIATVDNPSNAIEWAMGEMLNQPEILKKATEELDRVVGKDRLVQESDIPNLDYVKACAREAFRLHPVAHFNVPHVAMEDTVIGDYFIPKGSWAVLSRYGLGRNPKTWSDPLKYDPERHMNEGEVVLTEHELRFVTFSTGRRGCVASLLGSCMTTMLLARMLQCFTWTPPANVSKIDLAETLDELTPATPISAFAKPRLAPHLYPTSP

SEQ ID NO: 2 - DNA, 1845 bp, Manihot esculenta
gttcagggcatatcaatatggccatgaacgtctccaccaccatcggtttacttaacgccacctccttcgcctcctcctcctccatcaacacggtcaagatcttgttcgtcaccctctttatttccattgttagtactattgtaaaacttcaaaagagtgctgctaacaaggaaggtagcaagaaactcccactccctcctggccctactccatggccactcatcggaaacatcccggaaatgatccggtacagacccacgtttcggtggattcaccaactcatgaaggacatgaacactgatatttgtctcattcgttttggaagaactaactttgttcctataagctgtcctgttcttgctcgtgaaatactaaaaaagaatgacgctatcttctctaacaggccaaagactctctctgcaaaatctatgagcggaggatacttgacaactattgtggtgccatacaatgaccaatggaagaaaatgaggaagatcttaacctcagagatcatttctccggccagacacaaatggctccatgataaaagagctgaggaggctgataatcttgtgttctacatccacaaccagttcaaagcaaataaaaatgtgaatttgagaacagccaccaggcattacggcgggaatgtgatcagaaaaatggtgttcagcaagagatacttcggcaagggaatgccggacggaggaccagggcctgaagaaatcgagcacattgatgccgttttcactgccttgaaatacttgtatgggttttgcatatcagatttcttgcctttcttgttgggacttgatctggatggccaagaaaaatttgtgcttgatgcaaataagaccataagggattatcagaaccctttaattgatgaaaggattcaacaatggaagagtggtgaaaggaaggaaatggaggacttgcttgatgttttcatcactctcaaggattcagacggcaacccattgctcactcctgacgagatcaagaatcaaatagctgaaattatgatagcaacagtagataacccatcaaacgcaatcgaatgggcaatgggggagatgctaaatcaaccagaaatcctgaagaaggccacagaagagctcgacagggtggtcggcaaagacaggcttgttcaagaatccgacatccccaaccttgactatgtcaaagcctgtgcaagagaagccttcaggctccatccagtagcacacttcaatgtccctcatgtagccatggaagacactgtcattggtgattactttattccaaagggcagctgggcagttctcagccgctatgggctcggcaggaacccaaagacatggtctgatcctctcaagtacgatccagaaaggcacatgaacgagggagaggtggtgctcactgagcacgagttaaggtttgtgactttcagcactggaagacgtggctgcgtagcttcgttgcttggaagctgcatgacgacgatgttgctggcgaggatgctgcagtgcttcacttggactccaccagccaatgtttccaagattgatctcgccgagactctagatgagcttactcctgcaacacccatctctgcatttgccaagcctcgcctggctcctcatctctacccaacgtcaccttgaaagagagatcagatcttatcagttcttagaacgtcctttaattatgatttgctaaaaacaaataaaaatcatttggttattgtgtaggtaatcttacaagcttcctgtttattgagagttgttaattaactctcaaaatgatttgtggggttatcttgtttctcttgcaatatagttgctttactagaaaaaaaaaaaa

SEQ ID NO: 3 - protein, 541 aa, Manihot esculenta
MAMNVSTTATTTASFASTSSMNNTAKILLITLFISIVSTVIKLQKRASYKKASKNFPLPPGPTPWPLIGNIPEMIRYRPTFRWIHQLMKDMNTDICLIRFGKTNVVPISCPVIAREILKKHDAVFSNRPKILCAKTMSGGYLTTIVVPYNDQWKKMRKVLTSEIISPARHKWLHDKRAEEADQLVFYINNQYKSNKNVNVRIAARHYGGNVIRKMMFSKRYFGKGMPDGGPGPEEIMHVDAIFTALKYLYGFCISDYLPFLEGLDLDGQEKIVLNANKTIRDLQNPLIEERIQQWRSGERKEMEDLLDVFITLQDSDGKPLLNPDEIKNQIAEIMIATIDNPANAVEWAMGELINQPELLAKATEELDRVVGKDRLVQESDIPNLNYVKACAREAFRLHPVAYFNVPHVAMEDAVIGDYFIPKGSWAILSRYGLGRNPKTWPDPLKYDPERHLNEGEVVLTEHDLRFVTFSTGRRGCVAALLGTTMITMMLARMLQCFTWTPPPNVTRIDLSENIDELTPATPITGFAKPRLAPHLYPTSP

SEQ ID NO: 4 - DNA, 1920 bp, Manihot esculenta
ggtcttggtcatagccctggacttgaattgttcagggcaacaccaatatggccatgaacgtctccaccaccgcaaccaccacggcctccttcgcctccacgtcctccatgaacaatactgccaaaatcctccttatcaccctcttcatttccattgtcagtactgttataaaacttcaaaaaagggcatcctacaagaaagctagcaagaacttcccactccctcctggtccgactccatggccactcatcggaaacatccctgaaatgatccggtacagaccgacgtttcgttggattcaccaactcatgaaggacatgaacaccgatatttgtctgatccgtttcggaaaaactaacgttgttcctattagctgccctgtcattgctcgtgaaatcctgaaaaagcacgatgctgtcttctctaacaggccaaagattctctgcgctaaaacaatgagcggcggatacttgacgacgattgtggtgccatacaatgatcaatggaagaaaatgaggaaggtcctaacttcagagatcatttctccagctaggcacaaatggctccatgataagagagctgaggaagcagatcagcttgtgttctatatcaataaccagtacaagagcaacaagaatgtgaatgtgagaattgcggcaaggcattacggtggaaatgtgatcagaaagatgatgtttagcaagagatacttcggcaaagggatgcctgatggaggaccagggcctgaagaaatcatgcacgttgatgcaatttttacagcacttaaatatttgtatggattttgcatctctgattacttgccttttttggaggggcttgatcttgatggccaggaaaagattgtgcttaatgcaaataagaccataagggatcttcaaaacccattaatagaagaaaggattcaacaatggaggagtggtgaaagaaaggaaatggaagacttgcttgatgttttcattactcttcaggattcagatggcaagccattgctcaatccagacgagataaagaatcaaatcgctgaaattatgatagcaacaatagacaacccagcaaacgccgtagaatgggcaatgggggagctgataaatcaaccagaacttctggcaaaggccacagaggaacttgacagagtggtcggcaaagacaggcttgtgcaagaatctgacatccctaatcttaattacgtcaaagcctgtgcaagggaggccttcaggctccacccagttgcatacttcaacgtccctcacgtagccatggaagacgccgtcatcggcgattacttcattccaaagggcagctgggcaattcttagccgctacgggctcggccggaacccaaaaacatggcctgatccactcaagtacgacccagaaaggcacttgaacgagggcgaagtggtgctgactgagcacgaccttaggttcgtcacattcagcactggacgtcgtgggtgtgtcgctgctttgcttggaaccaccatgattacgatgatgctggccaggatgcttcagtgcttcacttggactccaccccctaatgtaaccaggattgatctcagtgagaatatcgatgagcttactccagcaacacccatcactggatttgctaagccacggttggctcctcatctctaccccacttcaccttgaattaaagcccaaagatgggaagggatgaatgtgagttgttagaagttttaataaaaaaattattgggtttatatgtgtaattacgtggtaaccttacaaagtgtctgttattgagagttttaatctctcaaaataatttgtgtggctaagatttcttcatctttgtatctcttgcaattgtttgctctataaaacatcttatttccttaaaaaaaaaaaaaaaaaaaaaaaaaaaaaaaaaaaaaaa

SEQ ID NO: 5 - DNA, 25 bp, Artificial Sequence
  modified_base (14): i
gcggaattcarggnaayccnytnct

SEQ ID NO: 6 - DNA, 26 bp, Artificial Sequence
  modified_base (18): i
cgcggatccggdatrtcngaytcytg

SEQ ID NO: 7 - DNA, 25 bp, Artificial Sequence
  Description of Artificial Sequence Oligonucleotide sequence
cgaaacgatggctatgaacgtctct

SEQ ID NO: 8 - DNA, 27 bp, Artificial Sequence
  Description of Artificial Sequence Oligonucleotide sequence
tggtagagacgttcatagccatcgttt

SEQ ID NO: 9 - protein, 540 aa, Triglochin maritima
MELITILPSVLPNIHSTATVLFLLLLTTALSFLFLFKQHLTKLTKSKSKSTTLPPGPRPWPIVGSLVSMYMNRPSFRWILAQMEGRRIGCIRLGGVHVVPVNCPEIAREFLKVHDADFASRPVTVVTRYSSRGFRSIAVVPLGEQWKKMRRVVASEIINAKRLQWQLGLRTEEADNIMRYITYQCNTSGDTNGAIIDVRFALRHYCANVIRRMLFGKRYFGSGGEGGGPGKEEIEHVDATFDVLGLIYAFNAADYVSWLKFLDLHGQEKKVKKAIDVVNKYHDSVIESRRERKVEGREDKDPEDLLDVLLSLKDSNGKPLLDVEEIKAQIADLTYATVDNPSNAVEWALAEMLNNPDILQKATDEVDQVVGRHRLVQESDFPNLPYIRACAREALRLHPVAAFNLPHVSLRDTHVAGFFIPKGSHVLLSRVGLGRNPKVWDNPLRFDPDRHLHGGPTAKVELAEPELRFVSFTTGRRGCMGGPLGTAMTYMLLARFVQGFTWGLRPAVEKVELEEEKCSMFLGKPLRALAKPRQELLQSF

SEQ ID NO: 10 - DNA, 1858 bp, Triglochin maritima
caatgcattgctcccactagcccactacgtactataaatgcatgcaccactccacctctcctcctcagtagcaaaatggaactcataaccattcttccatcagtgcttcctaacatccactctactgccacagtactgttcctcttgctactcaccacagccctctccttcctcttcctcttcaaacaacacctcactaagctaaccaagtccaagtccaagtccaccacattgccacccggcccccgaccatggcccatcgttggcagcctcgtgtcgatgtacatgaaccggccgtctttccggtggatactagcccagatggaggggagaaggatagggtgcattaggttgggtggtgttcatgttgttccggttaattgtcctgagattgctagggagtttcttaaggtgcatgatgctgattttgcatcgcgtccggtcacggttgtgactcgctactcgtctcgtgggttccggtctattgccgtggttccactgggggagcaatggaagaagatgaggagggtggtggcgtcggagattattaatgctaagaggctccaatggcagcttgggcttagaaccgaagaagccgacaacataatgaggtacatcacctaccaatgcaacacttcgggcgacactaacggagcgattatcgacgtccgcttcgccctccgccactactgtgccaatgtcatccggcgaatgctgttcgggaaacgctacttcggaagcggtggagaaggcggtgggccgggaaaggaggagattgagcacgttgacgccaccttcgacgtcttgggtctaatatacgccttcaatgcggcggactacgtgtcgtggttgaagttcttagacttgcatgggcaggagaagaaggttaagaaggccattgatgtggtgaataagtatcatgactccgttatcgagtcgaggagggagaggaaagtagagggaagagaggacaaggatccagaggatcttcttgatgtgcttttgtcgcttaaggattctaatgggaagcctctcttggacgtggaggagatcaaagcacaaattgcggatttgacgtacgcaacagttgataacccgtcgaacgccgtggaatgggcactagccgagatgctgaacaacccggacatcctccaaaaggcgaccgacgaggtagaccaggtcgtcggaaggcaccgtctcgtacaagaatccgacttcccgaacctcccctacatccgggcctgcgcccgggaggccctccgtctccaccctgtcgcggccttcaacctcccccacgtgtcccttcgtgacactcatgtcgccggttttttcattccaaaaggcagccacgttctcctgagtcgcgtcggcctcggacgcaaccccaaggtctgggacaacccgcttcgattcgaccccgaccgacacctccacggcgggcccaccgccaaagtcgagctggccgagccggagctgaggttcgtgtcgttcaccaccgggaggagagggtgcatggggggcccacttgggactgccatgacttatatgctgcttgctaggttcgtccagggtttcacttggggtcttcgccctgctgtggagaaggttgagcttgaggaggagaagtgtagcatgttcttgggcaagccattaagggctttggctaagccacgtcaggagctgctccagagcttctaattagggttagggtttgggttggattaataatacttatgaaatgcacgtttatgagtctataaatattatccatgtaagtgttatatgttttcgtgcaatcctattatccatgtaagttaaatttgataccatgaatgagtttatatgtgaaaaaaaaaaa

SEQ ID NO: 11 - protein, 533 aa, Triglochin maritima
LITILPSVLPNIHSSATLFLLLLMTTALSFLFLFKQHLAKLTKPKSTTLPPGPRPWPIVGSLVSMYMNRPSFRWILAQMEGRRIGCIRLGGVHVVPVNCPEIAREFLKVHDSDFASRPVTVVTRYSSRGFRSIAVVPLGEQWKKMRRVVASEIINAKRLQWQLGLRTEEADNIVRYITYQCNTSGDTSGAIIDVRFALRHYCANVIRRMLFGKRYFGSGGVGGGPGKEEIEHVDATFDVLGLIYAFNAADYVSWLKFLDLHGQEKKVKKAIDVVNKYHDSVIDARTERKVEDKDPEDLLDVLFSLKDSNGKPLLDVEEIKAQIADLTYATVDNPSNAVEWALAEMLNNPAILQKATDELDQVVGRHRLVQESDFPNLPYIRACAREALRLHPVAAFNLPHVSLRDTHVAGFFIPKGSHVLLSRVGLGRNPKVWDNPLQFNPDRHLHGGPTAKVELAEPELRFVSFTTGRRGCMGGLLGTAMTYMLLARFVQGFTWGLHPAVEKVELQEEKCSMFLGEPLRAFAKPRLELLQSF

SEQ ID NO: 12 - DNA, 1778 bp, Triglochin maritima
ctcataaccattcttccatcagtgctaccaaacatccactcttctgccacattgttcctcttgctactcatgaccacagccctctccttcctcttcctcttcaaacaacacctcgctaagctaaccaaacccaagtccaccacattgccacctggcccccgaccctggcccatcgttggcagcctcgtgtcgatgtacatgaaccggccgtccttccggtggatactagcccagatggaggggaggaggatagggtgcattaggttgggtggtgttcatgttgttccggttaattgtcctgagattgctagggagtttcttaaggtgcatgattctgattttgcatcgcgtccggtcacggttgtgactcgctactcgtctcgtgggttccggtctattgccgtggttccactgggggagcagtggaagaagatgaggagggtggtggcatcggagattattaatgctaagaggctccaatggcagcttgggcttagaaccgaagaagccgacaacatagtgaggtacatcacctaccaatgcaacacttcgggcgacactagcggagcgattatcgacgtccgcttcgccctccgccactactgtgccaatgtcatccggcgaatgctgttcggaaaacgctactttggtagcggtggagtaggcggtgggcctggaaaggaggagattgagcacgttgacgccaccttcgacgtcttgggtctaatatacgccttcaatgcggcggactacgtgtcgtggttgaagttcttagacttgcatgggcaggagaagaaggttaagaaggccattgatgtggtgaataagtatcatgactccgttatcgacgcgaggacagagagaaaagtggaggataaggatccagaggatcttcttgatgtgcttttttcgcttaaggattctaatggaaagcctctcttggacgtggaggagatcaaagcacaaattgcggatttgacgtacgcaacagttgacaacccgtcgaacgccgtggaatgggcactagccgagatgctgaacaacccggccatcctccaaaaggcgaccgacgagctagaccaggtcgtcggaaggcaccgtctcgtacaagaatccgacttcccgaacctcccctacatccgtgcctgcgcccgggaggccctccgtctccacccggtcgcggctttcaacctcccccacgtgtcccttcgtgacactcacgtcgccggcttctttattcccaaaggcagccacgttctcctgagtcgcgttggcctcggacgcaaccccaaggtgtgggacaacccgcttcaattcaacccagaccgacacctccacggcgggcccaccgccaaagtcgagctggccgaaccggagctgaggttcgtgtcgttcaccaccgggaggagagggtgcatggggggcctacttgggactgccatgacttatatgctgcttgctaggttcgtccagggtttcacttgggggcttcaccctgctgtggagaaggttgagcttcaggaggagaagtgtagcatgttcttgggcgagccattgagagcttttgctaagccacgtctggagctgctccagagcttctaattagttttggattaataataactataattactaccgatgtccttaaagttgcatgtcgtgtaactagcacttgttatatttatagttatgaaaggtacgtttatgaatctataaaaattatccatgtaattgttatatgttttcgtgcaatcgtattgtgagtttggtttacaaaa

SEQ ID NO: 13 - DNA, 26 bp, Artificial Sequence
  Description of Artificial Sequence primer
gcggaattcgayaayccnwsnaaygc

SEQ ID NO: 14 - DNA, 28 bp, Artificial Sequence
  Description of Artificial Sequence primer
gcggatccgcnacrtgnggnahrttraa

SEQ ID NO: 15 - DNA, 27 bp, Artificial Sequence
  Description of Artificial Sequence primer
gcggaattcwsnaaygcnrtngartgg

SEQ ID NO: 16 - DNA, 29 bp, Artificial Sequence
  Description of Artificial Sequence primer
gcggatccrttraannnngcnacnggrtg

SEQ ID NO: 17 - DNA, 30 bp, Artificial Sequence
  Description of Artificial Sequence primer
gcggaattccacacaggaaacagctatgac

SEQ ID NO: 18 - DNA, 29 bp, Artificial Sequence
  Description of Artificial Sequence primer
gcggatccagacgagtagcgagtcacaac

SEQ ID NO: 19 - DNA, 23 bp, Artificial Sequence
  Description of Artificial Sequence primer
gcggatccaagaggaacagtact

SEQ ID NO: 20 - DNA, 23 bp, Artificial Sequence
  Description of Artificial Sequence primer
gcggatccaagaggaacaatgtg

SEQ ID NO: 21 - DNA, 24 bp, Artificial Sequence
  Description of Artificial Sequence primer
gcgaatgcattgctcccactagcc

SEQ ID NO: 22 - DNA, 24 bp, Artificial Sequence
  Description of Artificial Sequence primer
gcgatggttatgagttccattttg

SEQ ID NO: 23 - DNA, 27 bp, Artificial Sequence
  Description of Artificial Sequence primer
gcgcatatggaactaataacaattctt

SEQ ID NO: 24 - DNA, 28 bp, Artificial Sequence
  Description of Artificial Sequence primer
gcgaagcttattagaagctctggagcag

SEQ ID NO: 25 - DNA, 51 bp, Artificial Sequence
  Description of Artificial Sequence primer
gcgcatatggctctgttattagcagtttttttcctcttcctcttcaaacaa

SEQ ID NO: 26 - DNA, 51 bp, Artificial Sequence
  Description of Artificial Sequence primer
gcgcatatggctcgtcaagttcattcttcttggaatttaccaccaggcccc

SEQ ID NO: 27 - protein, 6 aa, Artificial Sequence
  Description of Artificial Sequence primer encoded
DNPSNA

SEQ ID NO: 28 - protein, 7 aa, Artificial Sequence
  Description of Artificial Sequence primer encoded
FNXPHVA

SEQ ID NO: 29 - protein, 6 aa, Artificial Sequence
  Description of Artificial Sequence primer encoded
SNAVEW

SEQ ID NO: 30 - protein, 7 aa, Artificial Sequence
  Description of Artificial Sequence primer encoded
HPVAXFN

SEQ ID NO: 31 - protein, 7 aa, Artificial Sequence
  Description of Artificial Sequence primer encoded
VVTRYSS

SEQ ID NO: 32 - protein, 6 aa, Artificial Sequence
  Description of Artificial Sequence primer encoded
TVLFLL

SEQ ID NO: 33 - protein, 6 aa, Artificial Sequence
  Description of Artificial Sequence primer encoded
ATLFLL

SEQ ID NO: 34 - protein, 6 aa, Artificial Sequence
  Description of Artificial Sequence primer encoded
MELITI

SEQ ID NO: 35 - protein, 7 aa, Artificial Sequence
  Description of Artificial Sequence primer encoded
MELITIL

SEQ ID NO: 36 - protein, 5 aa, Artificial Sequence
  Description of Artificial Sequence primer encoded
LLQSF

SEQ ID NO: 37 - protein, 15 aa, Artificial Sequence
  Description of Artificial Sequence primer encoded
MALLLAVFFLFLFKQ

SEQ ID NO: 38 - protein, 15 aa, Artificial Sequence
  Description of Artificial Sequence primer encoded
MARQVHSSWNLPPGP

SEQ ID NO: 39 - protein, 523 aa, Arabidopsis thaliana
MLAFIIGLLLLALTMKRKEKKKTMLISPTRNLSLPPGPKSWPLIGNLPEILGRNKPVFRWIHSLMKELNTDIACIRLANTHVIPVTSPRIAREILKKQDSVFATRPLTMGTEYCSRGYLTVAVEPQGEQWKKMRRVVASHVTSKKSFQMMLQKRTEEADNLVRYINNRSVKNRGNAFVVIDLRLAVRQYSGNVARKMMFGIRHFGKGSEDGSGPGLEEIEHVESLFTVLTHLYAFALSDYVPWLRFLDLEGHEKVVSNAMRNVSKYNDPFVDERLMQWRNGKMKEPQDFLDMFIIAKDTDGKPTLSDEEIKAQVTELMLATVDNPSNAAEWGMAEMINEPSIMQKAVEEIDRVVGKDRLVIESDLPNLNYVKACVKEAFRLHPVAPFNLPHMSTTDTVVDGYFIPKGSHVLISRMGIGRNPSVWDKPHKFDPERHLSTNTCVDLNESDLNIISFSAGRRGCMGVDIGSAMTYMLLARLIQGFTWLPVPGKNKIDISESKNDLFMAKPLYAVATPRLAPHVYPT

SEQ ID NO: 40 - DNA, 1572 bp, Arabidopsis thaliana
atgctcgcgtttattataggtttgcttcttcttgcattaactatgaagcgtaaggagaagaagaaaaccatgttaattagccctacgagaaacctctctctccctcccgggccgaaatcttggcctttaatcggaaacctaccggaaatactagggaggaacaaaccggtgttccggtggatacattctctcatgaaagaactcaacaccgatattgcatgtatccgtcttgcgaatactcacgtgatccccgtgacatccccgagaattgcaagagagattctgaagaagcaagactccgttttcgccactagaccgctaacgatgggcacggagtactgcagccgcgggtacttgaccgttgcggtggagccacaaggagagcagtggaagaagatgaggagagtggtggcatctcacgtgacgagcaagaagagcttccaaatgatgctacaaaagagaaccgaagaggctgataacttagtccggtacatcaataaccgtagtgtcaaaaaccgtggtaatgcttttgtggttattgatttaaggcttgcggtacggcaatacagtggaaatgtagctcggaagatgatgtttggtataaggcattttggtaaaggaagtgaagatggatcgggaccagggttggaagagattgaacatgtggaatctttgtttacggttttaacccatctttacgcctttgcattgtcagattatgtcccgtggctaaggttcttggacttggaaggccatgagaaggttgtgagtaacgcaatgagaaatgtaagtaagtataacgacccttttgttgatgaaagactcatgcaatggcgaaatgggaagatgaaagaacctcaagattttcttgacatgtttataatagctaaagacactgacgggaagcctactctgtcggacgaagagatcaaagcacaagtgacggaactaatgttggcgacggttgataatccgtctaacgcggcagagtggggtatggcggagatgattaacgagccgagcatcatgcaaaaagccgtggaagagattgatagggtagttggaaaagaccgtcttgtcattgagtctgatctcccaaatcttaactatgtgaaggcttgtgtgaaagaagcattccggttacaccccgtggcaccgttcaacctccctcacatgtccaccactgatactgtggtagacggttatttcatccccaagggaagccacgtattgattagtcgtatggggattgggagaaatcctagtgtgtgggacaagccgcataagttcgaccctgagagacatttgagcactaacacatgtgtggatctaaacgagtctgatctgaatataatatcgttcagtgcaggacgaagaggttgtatgggtgtggacattgggtcagccatgacgtacatgttactggctcggttgattcaaggattcacgtggttaccagtgcctggtaagaataagattgatatttcagaaagcaagaatgatctttttatggcaaaaccattatacgcggttgccacacctcgtttagctccacatgtgtatccaacctaa

SEQ ID NO: 41 - DNA, 27 bp, Artificial Sequence
  Description of Artificial Sequence PCR primer A2F1
gtgcatatgcttgactccaccccaatg

SEQ ID NO: 42 - DNA, 28 bp, Artificial Sequence
  Description of Artificial Sequence PCR primer A2R1
atgcatttttctagtaatctttacgctc

SEQ ID NO: 43 - DNA, 37 bp, Artificial Sequence
  Description of Artificial Sequence PCR primer A2F2
cgtgaattccatatgctcgcgtttattataggtttgc

SEQ ID NO: 44 - DNA, 29 bp, Artificial Sequence
  Description of Artificial Sequence PCR primer A2R2
cggaagcttattaggttggatacacatgt

SEQ ID NO: 45 - DNA, 24 bp, Artificial Sequence
  Description of Artificial Sequence PCR primer A2R3
cgtcacttgtgctttgatctcttc

SEQ ID NO: 46 - DNA, 24 bp, Artificial Sequence
  Description of Artificial Sequence PCR primer A2F3
gaactaatgttggcgacggttgat

SEQ ID NO: 47 - DNA, 57 bp, Artificial Sequence
  Description of Artificial Sequence PCR primer A2FX1
cgtgaattccatatggctctgttattagcagtttttctcgcgtttattataggtttg

SEQ ID NO: 48 - DNA, 57 bp, Artificial Sequence
  Description of Artificial Sequence PCR primer A2FX2
cgtgaattccatatggctctgttattagcagtttttcttcttcttgcattaactatg

SEQ ID NO: 49 - DNA, 30 bp, Artificial Sequence
  Description of Artificial Sequence PCR primer A2R4
catctcgagtcttcttccactgctctcctt

SEQ ID NO: 50 - DNA, 17 bp, Artificial Sequence
  Description of Artificial Sequence PCR primer A2FX3
ttaatcggaaacctacc

SEQ ID NO: 51 - DNA, 33 bp, Artificial Sequence
  Description of Artificial Sequence PCR primer 17AF
cgtgaattccatatggctctgttattagctgtt

SEQ ID NO: 52 - DNA, 18 bp, Artificial Sequence
  Description of Artificial Sequence PCR primer A1R
gggccacggcacgggacc

SEQ ID NO: 53 - DNA, 2702 bp, Arabidopsis thaliana
ctcgagctcagtttcttcttcttcctcgtacttatcctcctcagccaaacgatctctcaccgtattctctagctgcactccgtactgagctccttttatctcctttatcaccaccactcttataaccttctccatctccgctgaaaaatgtataatagtaagcagaggaaccggttcaatttcgttggacacgtacttaaccagattaattaagtaaaccggagtttaaccagttgaatcaaagtaaaccaaaataagaagccaaaccaaataatgtatttattgaaccacgtagtctccatctaaaccagagaaccctaattcaaattttgatttgaaaacatggactaattaagattaccggaggcaagggcgtcgaagaagtcatcatcgccggcgagttcttttccggttttgcctttccagttatcaagatgtgttttaacatctgaaggatctaagtaaactccgatcgcagtgaacttcacttgaagaaagtggatctcaatgtctgtgatccctataacaagaatatgaacaatccatataaaattattgttacctgcgattttgttatgtatcgcattataaggtatcagacattaagaaagcaaaaaagaaataaaaaccttggcccagaagagagagtggcttggaagtgatgatctgtggaggaaaaggaacctcgtgaaccatgaccatctctgttcccactgtttgataaacaagaacacacaaatcttaggaaaaaaacaaagcattgaaaaaaagacaatgagaataattgaaacttgttagaactgaaaatcttactttagtggataaacttgtaataaaaaagaatgcaaagagtgtaagacttactttctaatttatattattttgaatctgagagtgaagaaatttataaatggcttggtgtactattttacgatcttagagaaacaatatcgaaattgtaaatgtgaatatctctctctatataataagccagggactggtggtaggtaacataattttgctaacgttcaaagcttgtgatctaaaagacgacgtattctttttatgaattcaatttttttgctaccaaagcttgtgatctcaattgtttgttagcgacccaccaggaagccacgtgtttggatcaagcactcagtccacaaccactcattctacctaacaaatgaaggtatagaagtataataattaaaagagatagaagaaagaattgctatgatacagtaaaaagagatcagatgtcaaatgtgaaacaaagcgtacataaattagatacaaaattagaagcagccacatttctccacaacggctcttgaaatcagtaacgtaaagtaaactgatgatgacaaagacccaaaaaaaaaaaaaaaaaaaaaaaagagaaataaagagtgtctttaaagcagtaacgtataaaaaccctttttcgtcttctcttctatctcgacctcccaaatcatgaaaggatcaattcatgactccgctattacgggtttagaggctcagcttatggcatcgaagcgaggactaatcgaagccgtgagtttggggatcactcataagtcatatctcaatctattgcgatcattatcacatttttgaactggtaagtaagtgttactgctgcaaatcgaaactgactattgaaagctatgcccatctttcgacacataaactaagagccaagtgggaacaaaggatcgaagagacaactgaaagagattgggtaatgtgtgcaaagtgccaaaaattggcttcagcaagtcatggtataatctctattctctaatcacaatctctagcttttcttaattagtccttatgtaatttgattatgttttaattcgcctcctaattaatttcatggttgatggatagtcgtgggtattccttttgctacgcatgtcgagccgaatggaagctgctaggattaaatttacagaagctgaatcaatttttaagtgggccaaatatttacagtttttataagcccaaatctccatgtccatattgtttttaacgtggcgctacctaaaaggggataaagatttcataaacagcattaacaatttaacatcaacaagattttaaagggataaggattaaggaatcgtaagcaaatttatccttagagattagatttagacgaatttggaaaagtaaaaagttggtaattaaatagaaatgtacttaaaacacaacatgtaatacattagacatatgagctgttgaaaaatcgtggtttttctaatgatggcgctacctaaaagggacaaggatttcataatgatgcattaccaatttaacatccacaagatttataagggataaggaataatcaaagaaaaaaacatgtcttacatatgagctgttgaaaaatcgtggattcatttaacattgttttcttcaacatttaaagcacatttattttccatagattacacttaaacaaaagcatttgtttcatggctataaatagcttattcctcatcatagataagaaaaaaccttttcgaactcaaataatttctccaaattgagatttaaaaaaaaaaatgcttgactccaccccaatgctcgcgtttattataggtttgcttcttcttgcattaactatgaagcgtaaggagaagaagaaaaccatgttaattagccctacgagaaacctctctctccctcccggg

SEQ ID NO: 54 - protein, 541 aa, Arabidopsis thaliana
MNTFTSNSSDLTTTATETSSFSTLYLLSTLQAFVAITLVMLLKKLMTDPNKKKPYLPPGPTGWPIIGMIPTMLKSRPVFRWLHSIMKQLNTEIACVKLGNTHVITVTCPKIAREILKQQDALFASRPLTYAQKILSNGYKTCVITPFGDQFKKMRKVVMTELVCPARHRWLHQKRSEENDHLTAWVYNMVKNSGSVDFRFMTRHYCGNAIKKLMFGTRTFSKNTAPDGGPTVEDVEHMEAMFEALGFTFAFCISDYLPMLTGLDLNGHEKIMRESSAIMDKYHDPIIDERIKMWREGKRTQIEDFLDIFISIKDEQGNPLLTADEIKPTIKELVMAAPDNPSNAVEWAMAEMVNKPEILRKAMEEIDRVVGKERLVQESDIPKLNYVKAILREAFRLHPVAAFNLPHVALSDTTVAGYHIPKGSQVLLSRYGLGRNPKVWADPLCFKPERHLNECSEVTLTENDLRFISFSTGKRGCAAPALGTALTTMMLARLLQGFTWKLPENETRVELMESSHDMFLAKPLVMVGDLRLPEHLYPTVK

SEQ ID NO: 55 - DNA, 1916 bp, Arabidopsis thaliana
gtcgacccacgcgtccgcaacagaaaccacaacaaaaactttgagtcctcttcttctctatacacaaacatgaacacttttacctcaaactcttcggatctcactaccactgcaaccgaaacatcgtcctttagcaccttgtatctcctctcaacacttcaagcttttgtggctataaccttagtgatgctactcaagaaattgatgacggatcccaacaaaaagaaaccgtatctgccaccgggtcccacaggatggccgatcattggaatgattccgacgatgctaaagagccggcccgttttccggtggctccacagcatcatgaagcagctcaatactgagatagcatgcgtgaagttaggaaacactcatgtgatcaccgtcacgtgccctaagatagcacgtgagatactcaagcaacaagacgctctcttcgcgtcgaggcctttaacttacgctcagaagatcctctctaacggctacaaaacctgcgtgatcactccctttggtgaccaattcaagaaaatgaggaaagttgtgatgacggaactcgtatgtccagcgagacacaggtggctccaccagaagagatcagaagaaaacgatcatttaaccgcttgggtatacaacatggttaagaactcgggctctgtcgatttccggttcatgactaggcattactgtggaaatgcaatcaagaagcttatgttcgggacgagaacgttctctaagaacactgcacctgacggtggacccaccgtagaagatgtagagcacatggaagcaatgtttgaagcattagggtttaccttcgctttttgcatctctgattatctgccgatgctcactggacttgatcttaacggtcacgagaagattatgagagaatcaagtgcgattatggacaagtatcatgacccaatcatcgacgagaggatcaagatgtggagagaaggaaagagaactcaaatcgaagattttcttgatattttcatctctatcaaagacgaacaaggcaacccattgcttaccgccgatgaaatcaaacccaccattaaggagcttgtaatggcggcgccagacaatccatcaaacgccgtggaatgggccatggcggagatggtgaacaaaccggagattctccgtaaagcaatggaagagatcgacagagtcgtcgggaaagagagactcgttcaagaatccgacatcccaaaactaaactacgtcaaagctatcctccgcgaagctttccgtctccatcccgtcgccgccttcaacctcccccacgtggcactttctgacacaaccgtcgccggatatcacatccctaaaggaagtcaagtccttcttagccgatatgggctgggccgtaacccaaaagtttgggccgacccactttgctttaaaccggagagacatctcaacgaatgctccgaagttactttgaccgagaacgatctccggtttatctcgttcagtaccgggaaaagaggttgtgcggctccggcgctaggaacggcgttgaccacgatgatgctcgcgagacttcttcaaggtttcacttggaagctacctgagaatgagacacgtgtcgagctgatggagtctagtcacgatatgtttctggctaaaccgttggttatggtcggtgaccttagattgccggagcatctctacccgacggtgaagtgagatgagacgacgccgtatatattttatgaaactacttttatataatcgcccaaccaagtttggtcaattccggttaccagaagataattggtcaaattgtgaacaaacttgtgtgttggtttcttggttctttttgggacacttgaattgtgtctcctttacctcttcttttgttgttttcaataaaaacttttattaccatttcaaaaaaaaaaaaaaaa

SEQ ID NO: 56 - DNA, 1974 bp, Arabidopsis thaliana
atgaacacttttacctcaaactcttcggatctcactaccactgcaaccgaaacatcgtcctttagcaccttgtatctcctctcaacacttcaagcttttgtggctataaccttagtgatgctactcaagaaattgatgacggatcccaacaaaaagaaaccgtatctgccaccgggtcccacaggatggccgatcattggaatgattccgacgatgctaaagagccggcccgttttccggtggctccacagcatcatgaagcagctcaatactgagatagcatgcgtgaagttaggaaacactcatgtgatcaccgtcacgtgccctaagatagcacgtgagatactcaagcaacaagacgctctcttcgcgtcgaggcctttaacttacgctcagaagatcctctctaacggctacaaaacctgcgtgatcactccctttggtgaccaattcaagaaaatgaggaaagttgtgatgacggaactcgtatgtccagcgagacacaggtggctccaccagaagagatcagaagaaaacgatcatttaaccgcttgggtatacaacatggttaagaactcgggctctgtcgatttccggttcatgactaggcattactgtggaaatgcaatcaagaagcttatgttcgggacgagaacgttctctaagaacactgcacctgacggtggacccaccgtagaagatgtagagcacatggaagcaatgtttgaagcattagggtttaccttcgctttttgcatctctgattatctgccgatgctcactggacttgatcttaacggtcacgagaagattatgagagaatcaagtgcgattatggacaagtatcatgacccaatcatcgacgagaggatcaagatgtggagagaaggaaagagaactcaaatcgaagattttcttgatattttcatctctatcaaagacgaacaaggcaacccattgcttaccgccgatgaaatcaaacccaccattaaggtatttatcacgttcctttcatataaggtttcgatcgtaaaaatatcaaaagaacaatttttgttaaattttatttgagaaagcatgcatatcaaatttatttacacatactaacattttgattcataaaacatttataaaagaagaaagaaacattttgtggtaaaagttgattagttacaatatttgttttttttttgctaaacatgggctacttttttgtttgtctcttttgattactttggtcaaagacagatgcatgcaacttaattgtatttatttttatgttatacaaaaattaaagatccaaaattaataaaagctggtatatatgtttataatgaataggagcttgtaatggcggcgccagacaatccatcaaacgccgtggaatgggccatggcggagatggtgaacaaaccggagattctccgtaaagcaatggaagagatcgacagagtcgtcgggaaagagagactcgttcaagaatccgacatcccaaaactaaactacgtcaaagctatcctccgcgaagctttccgtctccatcccgtcgccgccttcaacctcccccacgtggcactttctgacacaaccgtcgccggatatcacatccctaaaggaagtcaagtccttcttagccgatatgggctgggccgtaacccaaaagtttgggccgacccactttgctttaaaccggagagacatctcaacgaatgctccgaagttactttgaccgagaacgatctccggtttatctcgttcagtaccgggaaaagaggttgtgcggctccggcgctaggaacggcgttgaccacgatgatgctcgcgagacttcttcaaggtttcacttggaagctacctgagaatgagacacgtgtcgagctgatggagtctagtcacgatatgtttctggctaaaccgttggttatggtcggtgaccttagattgccggagcatctctacccgacggtgaagtga

SEQ ID NO: 57 - DNA, 17 bp, Artificial Sequence
  Description of Artificial Sequence primer T7
aatacgactcactatag

SEQ ID NO: 58 - DNA, 26 bp, Artificial Sequence
  Description of Artificial Sequence primer EST3
gctaggatccatgttgtatacccaag

SEQ ID NO: 59 - DNA, 20 bp, Artificial Sequence
  Description of Artificial Sequence primer EST6
cgggcccgttttccggtggc

SEQ ID NO: 60 - DNA, 24 bp, Artificial Sequence
  Description of Artificial Sequence primer EST7A
ggtcaccaaagggagtgatcacgc

SEQ ID NO: 61 - DNA, 44 bp, Artificial Sequence
  Description of Artificial Sequence primer 5′ ′native′ sense
atcgtcagtcgaccatatgaacacttttacctcaaactcttcgg

SEQ ID NO: 62 - DNA, 68 bp, Artificial Sequence
  Description of Artificial Sequence primer 5′ ′bovine′ sense
atcgtcagtcgaccatatggctctgttattagcagtttttacatcgtcctttagcaccttgtatctcc

SEQ ID NO: 63 - DNA, 45 bp, Artificial Sequence
  Description of Artificial Sequence primer 3′ ′end′ antisense
actgctagaattcgacgtcattacttcaccgtcgggtagagatgc

SEQ ID NO: 64 - DNA, 25 bp, Artificial Sequence
  Description of Artificial Sequence primer CYP79B2.2
ggaattcatgaacacttttacctca

SEQ ID NO: 65 - DNA, 27 bp, Artificial Sequence
  Description of Artificial Sequence primer B2SB
ttgtctagatcacttcaccgtcgggta

SEQ ID NO: 66 - DNA, 27 bp, Artificial Sequence
  Description of Artificial Sequence primer B2AF
ggcctcgagatgaacacttttacctca

SEQ ID NO: 67 - DNA, 27 bp, Artificial Sequence
  Description of Artificial Sequence primer B2AB
ttggaattccttcaccgtcgggtagag

SEQ ID NO: 68 - DNA, 31 bp, Artificial Sequence
  Description of Artificial Sequence primer Xba I
gtaccatctagattcatgtttgtgtatagag

SEQ ID NO: 69 - DNA, 2361 bp, Arabidopsis thaliana
gaattcattgatctggtcttgctaaaaactttaaaattgatgagttcaacatcttcaaatgcatgataacgggtccaacggaaattgacttttttttcatgctcctgatatataataatatctaacgattacgggttccactaattgtcattactcattaacattcctatttaaaagttgtgatagttttagggttttacgtagtcgtgtcatatagcgattaactacgtacttgtagatttatcaattacttctgttgtttacgagaacctaaaaaaaagaagcagatgcctagtttatagagcacgtgtactgtcttgaaaacttaggtaggttggtaaggttaccaaaagaccttaaaggaatataaagttactaattaacttaagtaaagttggtattgcttatatattgcaaagtattacaaaccaatcccctctgtatattgttttaaaccatagatttttttacaattaagtttatgatcaatcaattatttcaccatttctattaaattatgtaaaaagaaaaggatatatatatatatatataattaaataagaataaatcaaaataccgaaattttttattatccattctttgtggacatcgcccctaatatataaaaaaaaaaaactttcgtataactgatttatatttttttgtaaaaacttaaaggaagcctaagaaatatcttgtgatatttttgacaaaatgtggtatatatctttttataatatcatttataaagaaaatattgattacatggtgaaaaacattttgctagcgatcaacaaaattaaataggcacatgttaactgatctcatacgaccttgaaattttaatctttgtgtcgagagaccgatctttatgcaaattatgaaactacacatggtttatgcacggaagatcacattgcatgtataccatattataaaccaaaaatgatcaagaagaaggcgaaaacatttgggtaaattttaaatttcgatcatgcgattttttagctcatcatcaacagacaagaaactatcttttgtactgtaaatactaaatacaaaataaaatcttcatcattttttgcatgtgtcaaataaattacgcgaactttttttttttatcgactattaatagagaaacctgttttatttgccttgatttggaaaaatggagaaattgacttaagacttagtctcggtcacatcggcaacaacggagcttaaacggcgtccgcaacatggaaactcaagccacgaatctgatatattgactatagaagtagtaagtaactttgactcgtcccacatcagtttcaatttccacgagggtatttggcaggtgaactctctacgtacccaaaacataatggctattttatttcataactgatatttagcaattaattattcgtcctttttaaaccaatttctatagttgggaaaataatcaatttttacactttcaatgtatacgttacagatttttttttattagtcatgcacatattttcaatttttacactttcaatgtaaacaatcgattcttaattgttaaaaataggtttacgtaaggaattaaagatttgtttaaaatatgttccggccggtctaataatttacttgacgttaatttcttaaacacttttagataggaggctttgtttatcccaaatgattttgtaccactgcgacaatactagctagacataaaatgttaataaatttttattaagtaatataatcgaagtattagatcaatgtagtagacagttaggttaactaaaacaagagtaaacacttttttttttcttttcaggataggtaaaacaaatttcacactattttgcgtatttccttaaatttgttgttcgttttctcagcaaagatgaatattttgtttcatagtaattcacaagtataaactcgccagaactcctcaaacagtgaaatataatatagcttttaactgtttttcggctggaccgggtttttaagtgcatatataacacgaggaattttggcaggtcaccaacaaaacttttaaaaatattaaaaattcccatcaagaatagaaattaataaacaatgatatctctaataatatagatattttgaaacgttaggaataatcgtaataatgttcaacgttggtggtggtactcaagatggaccctccctcccacattttcctcactccttcgtaagtcctttccacgcataagggtattatagtcatttcacataaactaacgactactagacttgtatataaataggaaggtgaagctctctctttatccatgcagagacaacagaaaccacaacaaaaactttgagtcctcttcttctctatacacaaac

SEQ ID NO: 70 - protein, 540 aa, Brassica napus
MNTFTSNSSDLTSTTTQTSPFSNMYLLTTLQAFAAITLVMLLKKVFTTDKKKLSLPPGPTGWPIIGMVPTMLKSRPVFRWLHSIMKQLNTEIACVRLGNTHVITVTCPKIAREILKQQDALFASRPMTYAQNVLSNGYKTCVITPFGEQFKKMRKVVMTELVCPARHRWLHQKRAEENDHLTAWVYNLVKNSGSVDFRFVTRHYCGNAIKKLMFGTRTFSENTAPDGGPTAEDIEHMEAMFEALGFTFSFCISDYLPMLTGLDLNGHEKIMRDSSAIMDKYHDPIVDARIKMWREGKRTQIEDFLDIFISIKDEQGNPLLTADEIKPTIKELVMAAPDNPSNAVEWAMAEMVNKPEILHKAMEEIDRVVGKERLVQESDIPKLNYVKAILREAFRLHPVAAFNLPHVALSDATVAGYHIPKGSQVLLSRYGLGRNPKVWADPLSFKPERHLNECSEVTLTENDLRFISFSTGKRGCAAPALGTALTTMMLARLLQGFTWKLPENETRVELMESSHDMFLAKPLVMVGELRLPEHLYPTVK

SEQ ID NO: 71 - DNA, 1913 bp, Brassica napus
tggagctccaccgcggtggcggccgctctagaactagtggatcccccgggctgcaggaattcgcggccgcgtcgactttgattcttcttctctgctctctctctctctactcgaaaacatgaacacctttacctcaaactcttcggatctcacttccactacaacgcaaacgtctccgttcagcaacatgtatctcctcacaacgctccaggcctttgcggctataaccttggtgatgcttctcaagaaagtcttcacgacggataaaaagaaattgtctctcccgccgggtcccaccggatggccgatcatcggaatggttccaacgatgctaaagagccgtcccgttttccggtggctccacagcatcatgaagcagctaaacaccgagatagcctgcgtgaggctaggaaacactcacgtgatcaccgtcacatgcccgaagatagcacgtgagatactcaagcaacaagacgctctcttcgcctcgagacccatgacttacgcacagaatgtcctctctaacggatacaaaacatgcgtgatcactcccttcggtgaacaattcaagaaaatgaggaaagtcgtgatgactgaactcgtttgtcccgcgaggcacaggtggcttcaccagaagagagctgaagagaacgaccatttaaccgcttgggtatacaacttggtcaagaactctggctcagtcgattttcggtttgtcacgaggcattactgtggaaatgctatcaagaagcttatgttcgggacaagaacgttctctgaaaacaccgcacctgacggtggaccaaccgctgaggatatcgagcatatggaagctatgttcgaagcattagggtttactttctccttttgtatctctgattatctacctatgctcactggacttgatcttaacggccacgagaagatcatgagggattcgagtgctattatggacaagtatcacgatcctatcgtcgatgcaaggatcaagatgtggagagaaggaaagagaactcaaatcgaggattttctagacatttttatttctatcaaggatgaacaaggcaacccattgcttaccgccgatgaaatcaaacccaccattaaggaacttgtaatggcggcgccagacaatccatcaaacgctgtcgagtgggccatggcggagatggtgaacaaaccggagatactccataaagcaatggaagaaatagacagagttgtcggaaaagaaagacttgtccaagaatccgacattccaaaattaaattacgtcaaagctatcctccgtgaagccttccgcctccatcccgtagcggcctttaacctcccacacgtggcactttccgacgcaaccgtcgccgggtatcacatccctaaaggaagtcaagtccttctcagtcgatatgggctgggccgtaacccgaaagtttgggctgaccccttgagctttaaaccggagagacatctcaacgaatgctcggaagttactttgacggagaacgatctccggtttatctcgtttagtaccgggaaaagaggttgtgctgctccggctttaggtacggcgttgaccacgatgatgctcgcgagacttcttcaaggtttcacttggaagctgccggagaatgagacacgcgttgagctgatggagtctagccatgatatgtttttggctaaaccattggttatggtcggtgagttgagactcccagagcatctttacccgacggtgaagtaagaataaaacgacggcgtatatattttattaaataacttctacgtacttatgtaattaaccacagagtttggtcggtttctccggttaccagaagataatcggttaatatatgaacaaacttgtgcttggttttggtaaaaaaaaaaaaaaaaaaaaactcgagggggggccc

SEQ ID NO: 72 - DNA, 18 bp, Artificial Sequence
  Description of Artificial Sequence primer EST1
tccatgtgctctacatct

SEQ ID NO: 73 - DNA, 18 bp, Artificial Sequence
  Description of Artificial Sequence primer EST2
gacggaactcgtatgtcc

SEQ ID NO: 74 - protein, 537 aa, Arabidopsis thaliana
MSFTTSLPYPFHILLVFILSMASITLLGRILSRPTKTKDRSCQLPPGPPGWPILGNLPELFMTRPRSKYFRLAMKELKTDIACFNFAGIRAITINSDEIAREAFRERDADLADRPQLFIMETIGDNYKSMGISPYGEQFMKMKRVITTEIMSVKTLKMLEAARTIEADNLIAYVHSMYQRSETVDVRELSRVYGYAVTMRMLFGRRHVTKENVFSDDGRLGNAEKHHLEVIFNTLNCLPSFSPADYVERWLRGWNVDGQEKRVTENCNIVRSYNNPIIDERVQLWREEGGKAAVEDWLDTFITLKDQNGKYLVTPDEIKAQCVEFCIAAIDNPANNMEWTLGEMLKNPEILRKALKELDEVVGRDRLVQESDIPNLNYLKACCRETFRIHPSAHYVPSHLARQDTTLGGYFIPKGSHIHVCRPGLGRNPKIWKDPLVYKPERHLQGDGITKEVTLVETEMRFVSFSTGRRGCIGVKVGTIMMVMLLARFLQGFNWKLHQDFGPLSLEEDDASLLMAKPLHLSVEPRLAPNLYPKFRP

SEQ ID NO: 75 - DNA, 1614 bp, Arabidopsis thaliana
atgagctttaccacatcattaccatacccttttcacatcctactagtctttatcctctccatggcatcaatcactctactgggtcgaatactctcaaggcccaccaaaaccaaagaccgatcttgccagcttcctcctggcccaccaggatggcccatcctcggcaatctacccgaactattcatgactcgtcctaggtccaaatatttccgccttgccatgaaagagctaaaaacagatatagcatgtttcaactttgccggcatccgtgccatcaccataaactccgacgagatcgctagagaagcgtttagagagcgagacgcagatttggcagaccggcctcaacttttcatcatggagacaatcggagacaattacaaatcaatgggaatttcaccgtacggtgaacaattcatgaagatgaaaagagtgatcacaacggaaattatgtccgttaagacgttgaaaatgttggaggctgcaagaaccatcgaagcggataatctcatagcttacgttcactccatgtatcaacggtccgagacggtcgatgttagagagctctcgagggtttatggttacgcagtgaccatgcgaatgttgtttggaaggagacatgttacgaaagaaaacgtgttttctgatgatggaagactaggaaacgccgaaaaacatcatcttgaggtgattttcaacactcttaactgtttaccgagttttagtccagcggattacgtggaacgatggttgagaggttggaatgttgatggtcaagagaagagggtgacagagaactgtaacattgttcgtagttacaacaatcccataatcgacgagagggtccagttgtggagggaagaaggtggtaaggctgctgttgaagattggcttgatacgttcattaccctaaaagatcaaaacggaaagtacttggtcacaccagacgaaatcaaagctcaatgcgtagaattttgtatagcagcgattgataatccggcaaataacatggagtggacacttggggaaatgttaaagaacccggagattcttagaaaagctctgaaggagttggatgaagtagttggaagagacaggcttgtgcaagaatcagacataccaaatctaaactacttaaaagcttgttgtagagaaacattcagaattcacccaagtgctcattatgtcccttcccatcttgcgcgtcaagataccacccttgggggttatttcattcccaaaggtagccacattcatgtatgccgccctggactaggtcgtaaccctaaaatatggaaagatccattggtatacaaaccggagcgtcacctccaaggagacggaatcacaaaagaggttactctggtggaaacagagatgcgttttgtctcgtttagcaccggtcgacgtggctgcatcggtgttaaagtcgggacgatcatgatggttatgttgttggctaggtttcttcaagggtttaactggaaactccatcaagattttggaccgttaagcctcgaggaagatgatgcatcattgcttatggctaaacctcttcacttgtccgttgagccacgcttggcaccaaacctttatccaaagttccgtccttaa

SEQ ID NO: 76 - DNA, 42 bp, Artificial Sequence
  Description of Artificial Sequence primer sequence
ctctagattcgaacatatggctagctttacaacatcattacc

SEQ ID NO: 77 - DNA, 29 bp, Artificial Sequence
  Description of Artificial Sequence primer sequence
cgggatccttaaggacggaactttggata

SEQ ID NO: 78 - DNA, 29 bp, Artificial Sequence
  Description of Artificial Sequence primer sequence
aactgcagcatgatgagctttaccacatc

SEQ ID NO: 79 - DNA, 42 bp, Artificial Sequence
  Description of Artificial Sequence primer sequence
cgggatccttaatggtggtgatgaggacggaactttggataa

SEQ ID NO: 80 - DNA, 19 bp, Artificial Sequence
  Description of Artificial Sequence primer sequence
aaagctcaatgcgtagaat

SEQ ID NO: 81 - DNA, 29 bp, Artificial Sequence
  Description of Artificial Sequence primer sequence
tttttagacaccatcttgttttcttcttc

SEQ ID NO: 82 - DNA, 18 bp, Artificial Sequence
  Description of Artificial Sequence primer sequence
tgtagcggcgcattaagc

SEQ ID NO: 83 - DNA, 23 bp, Artificial Sequence
  Description of Artificial Sequence primer sequence
caaaagaatagaccgagataggg

SEQ ID NO: 84 - protein, 535 aa, Arabidopsis thaliana
MKISFNTCFQILLGFIVFIASITLLGRIFSRPSKTKDRCRQLPPGRPGWPILGNLPELIMTRPRSKYFHLAMKELKTDIACFNFAGTHTITINSDEIAREAFRERDADLADRPQLSIVESIGDNYKTMGTSSYGEHFMKMKKVITTEIMSVKTLNMLEAARTIEADNLIAYIHSMYQRSETVDVRELSRVYGYAVTMRMLFGRRHVTKENMFSDDGRLGKAEKHHLEVIFNTLNCLPGFSPVDYVDRWLGGWNIDGEEERAKVNVNLVRSYNNPIIDERVEIWREKGGKAAVEDWLDTFITLKDQNGNYLVTPDEIKAQCVEFCIAAIDNPANNMEWTLGEMLKNPEILRKALKELDEVVGKDRLVQESDIRNLNYLKACCRETFRIHPSAHYVPPHVARQDTTLGGYFIPKGSHIHVCRPGLGRNPKIWKDPLAYEPERHLQGDGITKEVTLVETEMRFVSFSTGRRGCVGVKVGTIMMAMMLARFLQGFNWKLHRDFGPLSLEEDDASLLMAKPLLLSVEPRLASNLYPKFRP

SEQ ID NO: 85 - DNA, 1608 bp, Arabidopsis thaliana
atgaagattagctttaacacatgctttcaaatcttactaggatttatcgtcttcatcgcatcaatcactttactaggtcgaatattctcaaggccttccaaaaccaaagaccggtgtcgccagcttcctcctggccgaccaggatggcccatcctcggcaatctacccgaactaatcatgactcgtcctaggtccaaatatttccaccttgccatgaaagagctaaaaacggatatcgcatgtttcaactttgccggaacccacaccatcaccataaactccgacgagatcgctagagaagcttttagagagcgagacgcagatttggcagaccggcctcaactttccatcgtagagtccattggagacaattacaaaacaatgggaacctcatcgtacggtgaacatttcatgaagatgaaaaaagtgatcacaacggaaattatgtccgttaaaacgttgaatatgttggaagctgcgagaaccatcgaagcggataatctcattgcttacattcactcgatgtatcaacggtcggagacggtcgacgttagagaactttcgagagtttatggttacgcagtgaccatgagaatgttgtttggaaggagacatgtcacgaaagaaaacatgttttcggatgatgggagactaggaaaagccgaaaaacatcatcttgaggtgattttcaacactctaaactgtttgccaggttttagtcccgtggattacgtggaccgatggttaggtggttggaatattgatggtgaagaggagagagcgaaagtgaatgttaatcttgttcgtagttacaacaatcccataatagacgagagggtcgaaatttggagggaaaaaggtggtaaggctgctgtggaagattggcttgatacgttcattacgctaaaagatcaaaacggaaactacttggttacgccagacgaaatcaaagctcaatgcgtcgaattttgtatagcagcgatcgataatccggcaaataacatggagtggacacttggggaaatgttaaagaacccggagattcttagaaaagctctgaaggagttggatgaagtagttggaaaagacaggcttgtgcaagaatcagacatacgaaatctaaactacttaaaagcttgttgcagagaaacattcaggattcacccaagcgctcattatgtcccacctcatgttgcccgtcaagataccacccttgggggttattttattcccaaaggtagccacattcatgtatgccgccctgggctaggccggaaccctaaaatatggaaagatccattagcatacgaaccggagcgtcacctccaaggagacggaatcacaaaagaggttactctggtcgaaacagagatgcgttttgtctcatttagcactggtagacgtggctgcgtcggtgtcaaagtcgggacaattatgatggctatgatgttggctaggtttcttcaaggttttaactggaaactccatcgagatttcggaccgttaagcctcgaggaagatgatgcatcattgcttatggctaagcctcttcttttgtctgttgagccacgcttggcatcaaacctttatccaaaattccgtccttaa